(19) United States
(12) Patent Application Publication
(10) Pub. No.: US 2018/0140720 A1
(43) Pub. Date: May 24, 2018

(54) COMPOSITIONS AND METHODS FOR TISSUE REGENERATION

(71) Applicant: Duke University, Durham, NC (US)

(72) Inventors: Kenneth D. Poss, Durham, NC (US); Junsu Kang, Durham, NC (US); Ravi Karra, Durham, NC (US)

(21) Appl. No.: 15/461,551

(22) Filed: Mar. 17, 2017

Related U.S. Application Data
(60) Provisional application No. 62/309,649, filed on Mar. 17, 2016.

(51) Int. Cl. A61K 48/00; C12N 5/10; C12N 5/074; C07K 14/46; C12N 15/861; A61P 9/00

(52) U.S. Cl. CPC A61K 48/005; C12N 5/10; C12N 5/0696; C07K 14/461; C12N 15/861; C12N 2015/8572; C12N 2750/14143; C12N 2510/00; C12N 2320/10; C12N 2800/106; A01K 2217/20; A61P 9/00

(57) ABSTRACT

The present disclosure relates to compositions and methods for tissue regeneration.

### CROSS-REFERENCE TO RELATED APPLICATIONS

This application claims priority to U.S. Provisional Patent Application No. 62/309,649, filed Mar. 17, 2016, the entirety of which is hereby incorporated by reference.

### STATEMENT REGARDING FEDERALLY SPONSORED RESEARCH OR DEVELOPMENT

This invention was made with Government support under Federal Grant No. R01 GM074057. The Federal Government has certain rights to this invention.

### FIELD OF THE INVENTION

The present disclosure illustrates that regeneration enhancer elements exist and can be isolated and provides guidance to new regeneration genes and tx regulators which may be relevant to the evolution of regenerative capacity. Further disclosed is that distinct tissues can engage different regeneration enhancers for the same gene and regeneration enhancers are minimal delivery modules for blocking/boosting regeneration with developmental factors. Finally, new tools are provided as well as enhancer delivery of regenerative factors.

### BACKGROUND OF THE INVENTION

Humans have a severely limited capacity to regenerate tissues like brain, heart, and limbs. Regenerative medicine aims to restore healthy tissue to structures like these, recapturing normal organ function. Proposed approaches to regenerative medicine include systemic administration of drugs, gene therapy using viruses or lipid vehicles, and cell therapy by systemic or local provision of stem cells. However, these approaches can suffer from low efficacy or potentially severe side effects. For example, the factor Neuregulin 1 can stimulate the division of cardiomyocytes, but also has the potential for neurological and oncogenic effects when delivered systemically. In addition, most gene regulatory elements used in gene therapy are constitutively active in all or specific cell types, but are not controlled in any way by the extent of injury. Thus, there are factors of safety and potential cytotoxicity through unwanted expression of genes in healthy tissues or continuing after the regeneration process is complete. Regulatory elements that activate only in injured tissue, and that ideally persistently activate expression until tissue is healed or regenerated, would provide a new level of specificity that could enhance gene therapy approaches to regenerative medicine.

Regenerative capacity is variable among species, tissues, and developmental stages. Whereas adult mammals do not regenerate cardiac muscle lost by ischemic myocardial infarction or amputated limbs, certain lower vertebrates like zebrafish regenerate these and other tissues like spinal cord very effectively. In a recent study, the inventors have established the concept of tissue regeneration enhancer elements, small DNA regulatory elements that help trigger regenerative programs in zebrafish. Importantly, it was also found that these elements can be engineered into simple DNA constructs that activate the production of pro-regenerative factors in injured and/or regenerative tissues in a manner that boosts regenerative capacity. These results support the idea that tissue regeneration enhance elements (TREEs) can be isolated from regenerating systems and engineered in a manner that can target therapeutic factors for regenerating tissue.

### SUMMARY OF THE INVENTION

The present disclosure provides, in part, compositions and methods for tissue regeneration. One aspect of the present disclosure provides a gene therapy construct comprising, consisting of, or consisting essentially of a nucleic acid encoding one or more tissue regeneration enhancer elements (TREEs) operatively linked to a promoter. In some embodiments, the gene therapy construct further comprises a nucleic acid sequence comprising one or more pro- or anti-regenerative factors, the expression of which is under the influence of the tissue regeneration enhancer elements.

In other embodiments, the gene therapy particle comprises a vector system. In some embodiments, the vector system comprises an AAV vector system.

In some embodiments, the gene therapy construct further comprises a first and second AAV inverted terminal repeat (ITR) sequence flanking the sequence encoding the TREEs. In certain embodiments, the ITR nucleotide sequences are derived from AAV serotype 2 (AAV-2).

Another aspect of the present disclosure provides a pharmaceutical composition comprising the gene therapy construct provided herein in a biocompatible pharmaceutical carrier.

In another embodiment, the TREE comprises the lepb-linked regulatory enhancer element (LEN).

One aspect of the present disclosure provides a method of treating or ameliorating tissue repair comprising, consisting of, or consisting essentially of administering to a subject a therapeutically effective amount of gene therapy construct as provided herein such that the tissue repair is treated or ameliorated.

Another aspect of the present disclosure provides a method of augmenting tissue regeneration in a subject comprising, consisting of, or consisting essentially of administering to the subject a therapeutically effective amount of a gene therapy construct as described herein such that the tissue regeneration is augmented.

Another aspect of the present disclosure provides a method of delivering cell therapy to a subject comprising, consisting of, or consisting essentially of inserting within the genome of reprogrammed stem/progenitor cells of the subject a gene therapy construct as provided herein such that cell therapy is delivered.

Another aspect of the present disclosure provides a method for screening drugs that modulate regenerative capacity comprising, consisting of, or consisting essentially of transfecting a gene construct as described herein into a model system, administering to the system a drug of interest, and measuring regenerative capacity in response to the drug.

In some embodiments, the model system comprises a zebrafish model system.

Another aspect of the present disclosure provides all that is disclosed and illustrated herein.

### BRIEF DESCRIPTION OF THE DRAWINGS

The foregoing aspects and other features of the disclosure are explained in the following description, taken in connection with the accompanying drawings, herein:

FIGS. 1A-1G are images showing activation of lepb regulatory sequences during tissue regeneration. FIGS. 1A and 1B. Regenerating heart (FIG. 1A) and fin (FIG. 1B) tissues. FIG. 1C. Genes with increased transcript levels in regenerating fins and/or hearts. lepb is in red (FC: fold-change). FIG. 1D. lepb:eGFP BAC transgenic construct, with the first exon replaced by eGFP. FIG. 1E. lepb:eGFP fluorescence (arrows) is detected in fins regenerating after amputation (dpa: days post-amputation). FIGS. 1F and 1G. lepb:eGFP fluorescence is undetectable in uninjured hearts (see also FIG. 7), but induced in regenerating hearts by 3 dpa. lepb:eGFP fluorescence (arrows in FIG. 1G) does not co-localize with MHC⁺ cardiomyocytes (FIG. 1F), but co-localizes with Raldh2⁺ endocardial cells (FIG. 1G). Antibodies detected: eGFP, MHC, and Raldh2 in FIGS. 1F and 1G. n=8; all animals displayed a similar expression pattern. Scale bars: e, 500 μm; FIGS. 1F and 1G, 50 μm.

FIGS. 2A-2C are images showing a DNA element upstream of lepb directs regeneration-dependent gene expression. FIG. 2A. Genomic DNA regions surrounding lepb, indicating RNA-seq and H3K27ac profiles from uninjured and regenerating hearts. Red bar, distal lepb-linked element enriched with H3K27ac marks (LEN). FIG. 2B. Transgene constructs examined for regeneration-dependent expression in fin or heart. EC, endocardial cells. FIG. 2C. (Top) Images of 2 dpa regenerating fins from transgenic reporter lines. Arrowhead, amputation plane. Arrows, blastemal eGFP. (Middle) Section images of resected ventricular region at 3 dpa. (Bottom) Atrial tissue distant from injury site. At least 5 fish from each transgeric line were examined, and all animals displayed a similar expression pattern. Arrows, endocardial eGFP. Scale bars: Top, 500 μm; Middle, 50 μm.

FIGS. 3A and 3B are images showing LEN activity in neonatal mice. FIG. 3A. Whole-mount (top) and section (bottom) images of X-gal stained hearts of LEN-hsp68::lacZ and Ctrl-hsp68::lac2 (control) lines, with clear staining in partially resected hearts of LEN-hsp68::lacZ mice (arrows) but not controls. n=5, 5, 6, and 4 for uninjured LEN-hsp68::lacZ, 3 days post-injury (dpi) LEN-hsp68::lacZ, uninjured control, and 3 dpi control hearts, respectively. Six sham-operated hearts showed minimal staining (see FIG. 12). Dashed red lines indicate injury area, positioned facing the front. Arrows, injury-dependent β-galactosidase expression. FIG. 3B. Whole-mount (left) and section (right) images of X-gal-stained digits from these lines, with X-gal staining detectable in amputated, but not uninjured, digits of LEN-hsp68::lacZ mice. n=14(7) and 12(6) for LEN-hsp68::lacZ and control digits (animals), respectively. Injuries were performed in neonatal mice on postnatal day 1 and assessed for expression on postnatal day 4. Arrowheads, injury planes. Arrows, injury-dependent β-galactosidase expression. P1, P2, P3, proximal, middle, and distal phalange, respectively. Scale bars: 1 mm.

FIGS. 4A-4C are images showing LEN is separable into tissue-specific elements. FIG. 4A. Transgene constructs to examine enhancer activation in regenerating fin or cardiac tissue. EC, endocardial cells. FIG. 4B. Regenerating fins (top) and sections of cardiac tissue from transgenic lines in a. Middle, resected ventricle region. Bottom, atrial tissue distant from injury site. At least 5 fish from each transgenic line were examined, and all animals displayed a similar expression pattern. Arrowheads, amputation plane. Arrows. blastimal (fin) or endocardial (heart) eGFP. FIG. 4C. Cartoon indicating separable tissue-specific regeneration modules in LEN. Scale bars: FIG. 4B, 50 μm.

FIGS. 5A-5G are images showing LEN controls fin regeneration when paired with Fgf effectors. FIG. 5A. Quantification of 3rd and 4th ray lengths from each lobe at 3 and 5 dpa. *P<0.01, One-way ANOVA; n=40 (10), 56 (14), and 40 (10) for wild-type, P2:dnfgfrl, and LENP2:dnfgfrl fin rays (animals), respectively. FIG. 5B. Representative Images of 5 dpa fin regenerates that were used for quantification of regenerate lengths in (FIG. 5A). Bottom, inset indicates dnfgfrl-eGFP fluorescence from boxed area (FIG. 5C). Images of 30 dpa LENP2-dnfgfrl fin regenerate. i, ii, eGFP fluorescence from boxed areas, maintained in impaired rays (right). FIG. 5D. Section ISH for fgf20a expression (arrows) in wild-type, dob, LENP2fgf20a, dob, and dob; P2:fgP20a fin regenerates at 3 dpa. FIG. 5E. 3 dpa fin regenerates from animals in FIG. 5D), stained for EdU incorporation (green) and nuclei (DAPI, blue), indicating extensive blastemnal proliferation in wild-type and dob; LENP2fgf20a regenerates. Fins were collected 60 minutes after EdU injection. FIG. 5F. Quantification of 3rd and 4th ray lengths from each lobe at 5 and 10 dpa. *P<0.01, One-way ANOVA; n=100 (25), 72 (18), 56 (14), and 100 (25) for wild-type, dob; LENP2:fgf20a, dob, and dob; P2:fgf20a fin rays (animals) at 5 dpa, respectively; n=98 (25), 72 (18), 56 (14), and 96 (24) at 10 dpa, respectively. FIG. 5G. Representative images of 5 dpa fin regenerates that were used for quantification of regenerate lengths in (FIG. 5F). The LENP2:fgf20a transgene rescues tin regeneration in dob animals, shown with controls at 5 dpa. Arrowheads in FIGS. 5B-5E and FIG. 5G, amputation planes. Scale bars: FIGS. 5B, 5C, and 5G, 500 μm; FIGS. 5D and 5E, 20 μm.

FIGS. 6A-6D are images showing enhancer-driven nrg1 expression boosts cardiomyocyte proliferation. FIG. 6A. Representative images of section ISH for nrg1 in P2:nrg1 (top) and LENP2:nrg1 (bottom) ventricles, at several times post-resection. P2:nrg1: n=4, 8, 7, and 3 for 3, 7, 14, and 30 dpa, respectively. LENP2:nrg1: n=4, 8, 8, and 4 for 3, 7, 14, and 30 dpa, respectively. Dashed lines, approximate resection planes. nrg1 (violet) is sharply induced in endocardial and epicardial cells in LENP2:nrg1 ventricular injuries. FIG. 6B. qPCR analysis of nrg1 in whole P2:nrg1 or LENP2:nrg1 cardiac ventricles at 3 dpa. FIG. 6C. Section images of 14 dpa regenerating ventricular apices from P2:nrg1 (top) and LENP2:nrg1 (bottom) animals, stained for cardiomyocyte nuclei (MEF2; red) and the proliferation marker PCNA (green). Insets indicated high-magnification view of regenerating area. Arrowheads, MEF2⁺PCNA⁺ cardiomyocytes. FIG. 6D. Quantified cardiomyocyte proliferation indices in injury sites in experiments from c. *P<0.01, Mann-Whitney rank sum test; n=11 (P2nrg1) and 15 (LENP2:nrg1). Scale bars: FIGS. 6A and 6C, 50 μm. Error bars indicate standard error.

FIGS. 7A-7M are images showing that lepb transcripts are sharply induced during fin and heart regeneration. FIG. 7A. Venn diagram displaying numbers of genes with significantly increased transcript levels during fin and heart regeneration. FIG. 7B. RT-PCR of samples from 2 days post-fertilization (dpf) and 4 dpf embryos, and uninjured and regenerating adult tissues. lepb was not detected during embryogenesis and in uninjured tissues, but induced during regeneration. β-act2 is used as loading control. Uninj, Uninjured. FIG. 7C. (Left) Relative expression of lepb in uninjured, 1, 2, and 4 dpa fin regenerates. lepb transcript levels are increased at 1 and 2 dpa. (Right) Relative expression of lepb in uninjured or 3 dpa cardiac ventricles, assessed by gPCR. FIGS. 7D and 7E. Endogenous lepb expression assessed by in situ hybridization in sections of fins (FIG. D) and cardiac ventricle and atrium (FIG. 7E). Arrowhead. amputation plane. Arrows, endocardial lepb expression. Left: uninjured tissues, Right regenerating tissues. dpa: days post-amputation. FIGS. 7F and 7G. F₀ animals, injected with the transgenic lepb:eGFP BAC reporter construct at the one-cell stage, induced eGFP after larval fin fold amputation (FIG. 7F) and during adult fin regeneration (FIG. 7G). Note that lepb:eGFP is mosaically expressed. Arrowheads, amputation planes. FIGS. 7H and 7I. Expression pattern of lepb:eGFP stable transgenic animals. lepb:eGFP was not detected in fin and heart during embryogenesis (FIG. 7H. 2 dpf, FIG. 7I, 4 dpf). Below ‘FIG. 7I’ are enlargements of the boxed areas, which show heart (left) and fin fold (right). Dotted line, outline of fin fold. The yolk is autofluorescent. FIGS. 7J and 7K. Section images of lepb:eGFP caudal fin regenerates at 2 dpa (FIG. 7J) and 4 dpa (FIG. 7K). The majority of lepb:eGFP-positive cells are mesenchymal cells, FIGS. 7L and 7M. Lack of detectable expression of lepb-eGFP in hearts of uninjured (FIG. 7L) or sham-operated (FIG. 7M) lepb:eGFP animals. n=8 and 5 for uninjured and sham-operated hearts, respectively. Arrowheads, amputation planes. Scale bars: FIGS. 7D, 7F, and 7H-7K, 10 μm; FIG. 7E, 1, FIG. 7M, 50 μm; FIG. 7G, 500 μm

FIGS. 8A-8J are images showing Leptin signaling during fin and heart regeneration. FIGS. 8A-8E. Expression pattern of lepr:lepr-mCherry BAC reporter line. FIG. 8A. Schematic of the lepr:lepr-mCherry BAC transgenic construct. mCherry is fused at the C-terminus of Lepr. FIG. 8B. mCherry fluorescence in the lepr:lepr-mCherry BAC reporter strain is induced during fin regeneration. n=5; all animals displayed a similar expression pattern. FIG. 8C. Section images of 4 dpa lepr:lepr-mCherry caudal fin regenerates. The majority of Lepr-mCherry⁺ cells are epidermal cells, overlapping partially with p63⁺ basal and suprabasal cells (left). In addition, some putative vascular cells in the intraray region have Lepr-mCherry signals (right FIGS. 8D and 8E. Confocal images of sections through uninjured (FIG. 8D) and regenerating (FIG. 8E) lepr:lepr-mCherry hearts. Lepr-mCherry fluorescence co-localizes with MHC⁺ cardiomyocytes in uninjured and 3 dpa hearts (arrows). Note that these expression patterns are similar to Leptin receptor expression in mice. n=7 and 6 for uninjured and 3 dpa hearts, respectively. FIGS. 8F-8J. Analysis of fin and heart regeneration in lepb^{pd94} mutants. FIG. 8F. A schematic representation of Lepb, showing the effects of the pd94 mutation. Lepb is composed of 5 alpha-helix domains. Lepb^{pd94} has a 19 bp insertion and a 3 bp deletion at the 3^{rd} α-helix (Helix C). FIG. 8G. Sequencing of wild-type and lepb^{pd94} alleles revealed an indel (Red highlight). FIG. 8H. A comparison of the amino acid sequences of Leptin genes in of human, mice, and zebrafish. The predicted amino acid sequence of the lepb^{pd94} gene product is shown at the bottom, with the predicted truncation sites indicated in red. The predicted lepb^{pd94} protein product lacks the majority of C-terminal amino acids. *Identical amino acid residue between three species. FIG. 8I. Quantification of regenerated fin lengths from lepb^{pd94} and wild type siblings at 4 dpa. n=12 each of lepb^{pd94} and wild-type. FIG. 8J. Quantification of cardiomyocyte proliferation at 7 dpa. n=7 (lepb^{pd94}) and 8 (wild-type). Data are represented as mean±SEM. N.S, Not significant.

FIGS. 9A-9F are images showing the identification of LEN and tests of regulatory sequences near lepb. FIG. 9A. Schematic depicting the genomic region surrounding lepb (corresponding to the lepb BAC used in this study) with the profiles of RNA-sequencing and H3K27ac marks from uninjured and regenerating heart tissues. FIG. 9B. Enlargement of the boxed area in a. lepb is the only upregulated gene in this genomic region during regeneration. H3K27ac-enriched peaks in regenerating samples are present in a ˜1 kb region (red bar) that is ˜7 kb upstream of the start codon. FIG. 9C. Schematic representation of transgenes to examine regulatory sequence activity. Fin and endocardial expression during regeneration and the number of stable lines are indicated. *One LENP2:eGFP line showed occasional, weak endocardial eGFP expression in uninjured hearts, whereas eGFP signal in this line was broad and strong during regeneration. EC, endocardial cells. FIG. 9D. Images of representative 0 dpa fins from lines indicated in (FIG. 9C). eGFP fluorescence is not detectable in fins at 0 dpa or in uninjured fins, but is induced in regenerating ray blastemas in P7:eGFP and LENP2:eGFP lines. P6:eGFP regenerates displayed weak eGFP expression below the amputation plane during regeneration, with very weak or undetectable expression in regenerating portions (see FIG. 2C). FIG. 9E. LENP2:eGFP expression pattern during fin regeneration. eGFP is detectable as early as 12 hpa, but is undetectable at 30 dpa. n=5; all animals displayed a similar expression pattern. Arrowheads, amputation planes. FIG. 9F. Section images of representative uninjured and regenerating hearts from P2:eGFP, P6:eGFP, P7:eGFP, and LENP2:eGFP animals. eGFP fluorescence is rarely detectable in uninjured P2:eGFP, P6:eGFP, P7:eGFP, or LENP2eGFP hearts, except in one line or LENP2eGFP (mentioned above). Upon injury, P2 drove weak, occasional expression in cardiomyocytes and epicardium but not in endocardium, whereas P7 and LEN drove endocardial eGFP expression in ventricle and atrium. i, ii, enlargements of boxes areas in regenerating ventricle and atrium, respectively. Scale bars: FIGS. 9D and 9E, 500 μm; FIG. 9F, 50 μM.

FIGS. 10A-10E are images showing additional putative regeneration enhancer elements. FIG. 10A. Cartoon depicting the distal upstream regions of i111a, cd248b, vcana, and plek. RNA-sequencing profiles indicate that these genes are upregulated during heart regeneration. The red bar indicates putative enhancer regions that are enriched with H3K27ac marks in regenerating tissue. Two of these putative enhancers, near illla and vcana, showed primary sequence conservation in other non-mammalian vertebrates but not in mammals. FIG. 10B. Scheme depicting assays in injected F₀ transgenic animals. At 4 dpf, eGFP expression in the uninjured fin fold was examined, and then the fin fold was amputated. eGFP expression near the amputation plane was examined at 5 dpf. FIG. 10C. Table indicating injected constructs and the number of animals with eGFP⁺ cells near amputation plane. FIG. 10D. Images of representative 4 dpf (uninjured) and 5 dpf (regenerating) fin folds from animals in (FIG. 10C). FIG. 10E. Vista plot of genomic regions from mir129 to lepb based on LAGAN alignment with reference sequence zebrafish. Sequence comparison indicates that this region is not highly conserved between zebrafish and mammals. Arrowheads, amputation planes.

FIGS. 11A and 11B are transient transgenic assays examining lepb-linked regeneration enhancer fragments in combination with different promoters (fin regeneration). FIG. 11A. Scheme depicting assays in injected F⁰ transgenic animals. Transgene-positive larvae were selected by detection of eGFP in response to fin fold amputation (lepb promoter), in cardiomyocytes (cmlc2 promoter), or in lenses (α-cry promoter). Caudal fins of F0 transgenic positive zebrafish were amputated at 60-90 days post-fertilization (dpf), and eGFP expression was examined at 2 dpa. FIG. 11B. Schematic representation of the transgenic constructs to examine fin regeneration enhancer activity. Expression during fin regeneration and the number of assessed F₀ animals are indicated. Many embryos transgenic for LEN(1-850), LEN(450-1000), LEN(450-850), and LEN(660-850) coupled with the lepb or cmlc2 promoter showed activity during fin regeneration. One of 11 LENα-cry:eGFP animals displayed fin eGFP expression, but LEN((1-850)α-cry:eGFP and LEN(450-1000) α-cry:eGFP did not drive eGFP expression during fin regeneration, indicating that there may be repressive motifs in the α-cry promoter fragment that affect fin regeneration enhancer activity (See also FIG. 15). N.D., not determined.

FIGS. 12A and B are images showing x-gal staining in stable transgenic mouse lines. FIG. 12A. Additional whole mount images of X-gal stained hearts from neonatal LEN-hsp68::lacZ (line 13, presented in FIG. 3) and control animals injured at postnatal day 1 and assessed at postnatal day 4. X-gal staining is undetectable in sham-operated hearts of LEN-hsp68::lacZ mice (n=6; representative image shown) and injured hearts of control mice, but strong in partially resected hearts of LEN-hsp68::lacZ mice (Arrows). Dashed red lines indicate injury area, positioned facing the front Arrows, injury-dependent β-galactosidase expression. dpi, days post injury. FIG. 12B. Whole mount images of X-gal stained hearts and paws from LEN-hsp68::lacZ line 6, which exhibited vascular endothelial expression in uninjured hearts and paws. Scale bars: 1 mm.

FIGS. 13A and 13B are transgenic assays examining lepb-linked regeneration enhancer fragments in combination with lepb P2 (fin regeneration). FIG. 13A. Schematic representation of the transgenic constructs to examine LEN fragments that drive expression during fin regeneration. Expression during fin regeneration and the number of stable lines is indicated. FIG. 13B. Images of representative 0 dpa and 2 dpa fins from a. eGFP fluorescence is rarely detectable in uninjured fins. LEN(1-850), LEN(450-1000), LEN(450-850), and LEN(660-850) coupled with P2 drove eGFP expression during fin regeneration. *LEN(830-1350)P2:eGFP lines exhibited very weak eGFP expression in fin regenerates, detectable with long exposure times and at high magnification (data not shown), suggesting the possibility of minor fin regeneration enhancer elements in 850-1000. At least 5 fish from each transgenic line were examined, and all animals displayed a similar expression pattern Arrowhead amputation planes.

FIGS. 14A-H are images of heart sections from uninjured and regenerating transgenic lines that employ lepb-linked enhancer fragments (FIGS. 14A-14H). eGFP fluorescence is rarely detectable in uninjured hearts in all transgenic lines. One exception is LEN(1000-1350)P2:eGFP, which showed occasional, weak endocardial eGFP expression in uninjured hearts. LEN(1-850)P2:eGFP (FIG. 14A), LEN(450-1000)P2eGFP (FIG. 14B), LEN(450-850)P2:eGFP (FIG. 14D), and LEN(660-850)P2:eGFP (FIG. 14G) transgenic lines, which include distal LEN elements, directed eGFP expression from promoters in a subset of epicardial cells and/or cardiomyocytes, but not endocardial cells. LEN(450-660)P2eGFP lines (FIG. 14E) showed regeneration-dependent enhancer activity in cardiomyocytes near the injury site, but not in endocardial cells. Our data indicated that the activities of LEN(1-850)P2:eGFP (FIG. 14A), LEN(450-1000)P2eGFP (FIG. 14B), and LEN(450-850)P2eGFP (FIG. 14D) lines were not as strong as LEN(450-660)P2:eGFP (FIG. 14E), suggesting that there might be repressive elements for cardiomyocyte expression outside of sequences 450-660. LEN(830-1350) (FIG. 14C) and LEN(1000-1350) (FIG. 14H), which did not activate expression from promoters during fin regeneration, could direct endocardial expression in both ventricle and atrium during regeneration, similar to the reference reporters lepb:eGFP and LENP2:eGFP. Arrows in FIGS. 14C and 14H: endocardial eGFP. i, ii: Enlargements of the boxed areas in regenerating ventricle and atrium, respectively. At least 5 fish from each transgenic line were examined, and all animals displayed a similar expression pattern. Scale bars, 50 μm.

FIGS. 15A-15J are transgenic assays examining lepb-linked enhancer fragment activity in combination with cmic2 and α-cry promoters. FIG. 15A. Schematic representation of the transgenic constructs to examine enhancer fragment activity in combination with the cmlc2 promoter. Expression during fin regeneration and the number of stable lines is indicated. FIG. 15B. Images of representative 0 dpa and 2 dpa fins from a. eGFP fluorescence was very weak or undetectable in 0 dpa or uninjured fins. (1-850), (450-1000), (450-850), and (660-850) LEN fragments coupled with the cmlc2 promoter activated blastemal eGFP fluorescence (arrows) during fin regeneration. One LEN(1-850)cmlc2:eGFP line did not show fin regeneration enhancer activity. Arrowheads, amputation planes. At least five fish from each transgenic line were examined, and all animals displayed a similar expression pattern except for the following: For two strains of LEN(450-850)cmlc2:eGFP, 4 of 5 animals induced eGFP fluorescence at 2 dpa; For LEN(660-850)cmlc2:eGFP, 4 of 7 animals induced eGFP fluorescence at 2 dpa. FIG. 15C. (Left) Schematic diagram of the LEN(1000-1350)cmlc2:eGFP transgenic construct. (Right) Images of sections from uninjured and regenerating LEN(1000-1350)cmlc2:eGFP hearts. eGFP is expressed mosaically in cardiomyocytes via the cmlc2 promoter. Uninjured hearts had no detectable endocardial eGFP fluorescence, whereas 3 dpa hearts displayed induced endocardial eGFP fluorescence (arrows). Arrowheads indicate cardiomyocyte eGFP fluorescence driven by cmlc2 promoter activity. FIGS. 15D-15H. Schematic representation of the transgenic constructs to examine enhancer fragment activity in combination with the α-cry promoter. Expression during fin regeneration and injury-activated endocardial expression, and the number of stable lines are indicated. At least 5 fish from each transgenic line were examined, and all animals displayed a similar expression pattern. EC, endocardial cells. One LENαcry:eGFP line showed regeneration-dependent expression (arrows) in fins (FIG. 15E) yet, unlike when coupled with lepb and cmlc2 promoters, the LEN(450-1000) fragment did not drive expression during fin regeneration (FIG. 15D and data not shown). This suggests a possible repressive motif within α-cry sequences. *One LENα-cry:eGFP line showed weak endocardial eGFP expression in uninjured hearts, but the eGFP signal (arrows) was stronger and broader during regeneration (FIG. 15G). Two LEN(830-1350)α-cry:eGFP lines had no detectable eGFP fluorescence in regenerating fins (FIG. 15F) uninjured hearts (FIG. 15H), but displayed induced endocardial eGFP fluorescence (allows) during heart regeneration (FIG. 15H). i, ii: Enlargements of the boxed areas in regenerating ventricle and atrium, respectively. FIG. 15I. LEN sequences annotated with putative binding sites in fin- (663-854) and cardiac- (1034-1350) regeneration enhancer modules. FIG. 15J. A predicated AP-1 binding site is necessary for fin regeneration enhancer activity. (Top) Schematic representation of the LEN(450-850-AP-lmut)P2 transgenic construct, in which the predicted AP-1 binding site (tgactca) is mutated to aaaaaa. Two LEN(450-850-AP-imut)P2 lines had no detectable eGFP fluorescence in regenerating fins. Scale bars, 50 μm.

FIGS. 16A-16F are images showing pairing LEN with potent developmental influences can control regenerative capacity. FIG. 16A. Images of representative F₀ transgenic zebrafish injected with P2:dnfgfr1 (left) or LENP2:dnfgfr1 (right) constructs, shown at 3 dpa. The dn-fgfr1 cassette is fused in frame to eGFP. Whereas zero of 27 P2dnfgfr F0 animals displayed defective regeneration, 7 of 67 LENP2:dnfgfr1 F₀ zebrafish had impaired fin regeneration in some fin rays, corresponding to eGFP fluorescence (arrow). FIG. 16B. Additional examples of LENP2:dnfgfr1 fins at 30 dpa, from experiments with a stable line. Inset in (FIG. 16B): high magnification view of the boxed area, showing eGFP fluorescence. FIG. 16C. Quantification of regenerated area from dob; LENP2:fgf20a F₀ transgenic zebrafish (n=45, 44 at 5, 10 dpa), dob mutants (n 9, 19 at 5, 10 dpa), and dob; P2fgf20a F₀ transgenic zebrafish (n=40, 40 at 5, 10 dpa) at 5 dpa and 10 dpa. Dotted line indicates 500,000 μm². FIG. 16D. images of representative dob; LENP2:fgf20a F₀ transgenic zebrafish, dob mutants, and dob; P2:gf20a F₀ transgenic zebrafish at 5 dpa. FIG. 16E. Confocal images of tissue sections of 3 dpa fin regenerates. Mosaic regenerates indicate expression of the linked eflanls-mCherry marker construct (red), and EdU incorporation (collected 60 minutes after injection; green). DAPI, blue. F₀ mosaic dob; LENP2fgf20a regenerates show evidence of distal growth and blastemal EdU incorporation. Arrow, blastemal. Dotted lines, amputation planes. i, ii, Enlargements of the boxed areas. FIG. 16F. In situ hybridization in sections of 3 dpa fin regenerates from dob; P2fgf20a (left) and F₀ mosaic dob; LENP2:fgf20a (right) animals, indicating LEN-induced fgf20a expression in mesenchymal cells and regenerative growth. Fgf20a is rarely detected in dob; P2:fgf20a regenerates. Arrowheads, amputation planes.

FIG. 17 shows that zebrafish merge elevated regenerative capacity with genetic tools.

FIG. 18 shows fin regeneration.

FIG. 19 demonstrates that resident osteoblasts contribute only new osteoblasts during fin regeneration.

FIG. 20 is a photograph illustrating fin regeneration 4 days post amputation in sde1 and sde1/+ zebrafish.

FIG. 21 illustrates the highly regenerative zebrafish heart.

FIG. 22 illustrates natural heart recognition in zebrafish.

FIG. 23 illustrates heart regeneration and the three major cardiac cell types.

FIG. 24 illustrates identified regeneration factors.

FIG. 25 illustrates factors controlling the induction and restriction of regeneration factors in fin and heart.

FIG. 26 illustrates a model for specialized enhancer regulatory elements serve to activate regeneration programs.

FIG. 27 discloses that leptin b is sharply induced during fin and heart regeneration.

FIG. 28 further illustrates that leptin b is sharply induced during fin and heart regeneration.

FIG. 29 discloses that a 150 kb region surrounding lepb drives no detectable expression in larvae or adults.

FIG. 30 discloses that regeneration elements are contained in a 150 kb region surrounding lepb.

FIG. 31 is a cartoon illustrating enhancer elements are identifiable by chromatin profiling.

FIG. 32 illustrates that lepb upstream sequences indicate open chromatin during heart regeneration.

FIG. 33 demonstrates that the lepb-linked element is required in reporter constructs for regeneration expression.

FIG. 34 demonstrates that the 1.3 kb lepb upstream sequence element directs regeneration expression in heart and fin.

FIG. 35 demonstrates that lepb-linked enhancer resolves to adjacent tissue-specific modules.

FIG. 36 demonstrates that lepb-linked enhancer modules activate expression from at least 3 promoters.

FIG. 37 illustrates definition of upstream binding factors and identification of additional enhancer elements.

FIG. 38 illustrates new enhancer-effector pairings can cause disease.

FIG. 39 illustrates the implication of enhancer adoption in autosomal dominant adult-onset leukodystrophy.

FIG. 40 queries whether enhancers can be engineered to modulate regenerative capacity.

FIG. 41 demonstrates that Fgf20a is a key mitogen for fin regeneration.

FIG. 42 demonstrates that lepb-linked enhancer targets a dominant-negative Fgf receptor to amputation site.

FIG. 43 demonstrates that lepb-linked enhancer delivery of a dominant-negative Fgf receptor impairs regeneration.

FIG. 44 illustrates lepb-linked enhancer targets Fgf ligand gene expression to amputation site.

FIG. 45 illustrates that lepb-linked enhancer delivery of ligand rescues regeneration.

FIG. 46 illustrates that neuregulin1 is induced by cardiac injury in zebrafish, mainly in the epicardial lineage.

FIG. 47 demonstrates the induced myocardial Nrg1 reactivation in adults causes massive cardiomyocyte proliferation.

FIG. 48 demonstrates continued cardiomyocyte hyperplasia during myocardial nrg1 reactivation.

FIG. 49 demonstrates lepb-linked enhancer constructs induce Neuregulin1 specifically in cardia injury sites.

FIG. 50 demonstrates lepb-linked enhancer delivery of Neuregulin1 boosts injury-induced cardiomyocyte division.

FIG. 51 is a graphic illustration of injury induction of LEN sequences in mice.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

Unless otherwise defined, all technical terms used herein have the same meaning as commonly understood by one of ordinary skill in the art to which this disclosure belongs.

### Definitions

As used herein, the terms “gene transfer,” “gene delivery,” and “gene transduction” refer to methods or systems for reliably inserting a particular nucleotide sequence (e.g., DNA) into targeted cells.

As used herein, the term “gene therapy” refers to a method of treating a patient wherein polypeptides or nucleic acid sequences are transferred into cells of a patient such that activity and/or the expression of a particular molecule is restored.

As used herein, the term “adenoviral associated virus (AAV) vector,” “AAV gene therapy vector,” and “gene therapy vector” refer to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, and AAV-6. The ITRs, however, need not be the wild-type nucleotide sequences, and may be altered (e.g., by the insertion, deletion or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (e.g., functional ITRs) of the virus.

The terms “adeno-associated virus inverted terminal repeats” or “AAV ITRs” refer to the palindromic regions found at each end of the AAV genome which function together in cis as origins of DNA replication and as packaging signals for the virus. For use in some embodiments of the present invention, flanking AAV ITRs are positioned 5′ and 3′ of one or more selected heterologous nucleotide sequences. Optionally, the ITRs together with the rep coding region or the Rep expression products provide for the integration of the selected sequences into the genome of a target cell.

As used herein, the term “AAV rep coding region” refers to the region of the AAV genome that encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Muzyczka (Muzyczka, Curr. Top. Microbiol. Immunol., 158:97-129 (1992)) and Kotin (Kotin, Hum. Gene Ther., 5:793-801 (1994)) provide additional descriptions of the AAV rep coding region, as well as the cap coding region described below. Suitable homologues of the AAV rep coding region include the human herpesvirus 6 (HHV-6) rep gene which is also known to mediate AAV-2 DNA replication (Thomson et al., Virol., 204:304-311 (1994)).

As used herein, the term “AAV cap coding region” refers to the region of the AAV genome that encodes the capsid proteins VP1, VP2, and VP3, or functional homologues thereof. These cap expression products supply the packaging functions, which are collectively required for packaging the viral genome. In certain embodiments, AAV2 Cap proteins may be used.

As used herein, the term “AAV helper function” refers to AAV coding regions capable of being expressed in a host cell to complement AAV viral functions missing from the AAV vector. Typically, the AAV helper functions include the AAV rep coding region and the AAV cap coding region. The helper functions may be contained in a “helper plasmid” or “helper construct.” An AAV helper construct as used herein, refers to a molecule that provides all or part of the elements necessary for AAV replication and packaging. Such AAV helper constructs may be a plasmid, virus or genes integrated into cell lines or into the cells of a subject. It may be provided as DNA, RNA, or protein. The elements do not have to be arranged co-linearly (i.e., in the same molecule). For example, rep78 and rep68 may be on different molecules. An “AAV helper construct” may be, for example, a vector containing AAV coding regions required to complement AAV viral functions missing from the AAV vector (e.g., the AAV rep coding region and the AAV cap coding region).

As used herein, the terms “accessory functions” and “accessory factors” refer to functions and factors that are required by AAV for replication, but are not provided by the AAV vector or AAV helper construct. Thus, these accessory functions and factors must be provided by the host cell, a virus (e.g., adenovirus or herpes simplex virus), or another expression vector that is co-expressed in the same cell. Generally, the E1, E2A, E4 and VA coding regions of adenovirus are used to supply the necessary accessory function required for AAV replication and packaging (Matsushita et al., Gene Therapy 5:938 (1998)).

Portions of the AAV genome have the capability of integrating into the DNA of cells to which it is introduced. As used herein, “integrate,” refers to portions of the genetic construct that become covalently bound to the genome of the cell to which it is administered, for example through the mechanism of action mediated by the AAV Rep protein and the AAV ITRs. For example, the AAV virus has been shown to integrate at 19q13.3-qter in the human genome. The minimal elements for AAV integration are the inverted terminal repeat (ITR) sequences and a functional Rep 78/68 protein. The present invention incorporates the ITR sequences into a vector for integration to facilitate the integration of the transgene into the host cell genome for sustained transgene expression. The genetic transcript may also integrate into other chromosomes if the chromosomes contain the AAV integration site.

The predictability of insertion site reduces the danger of random insertional events into the cellular genome that may activate or inactivate host genes or interrupt coding sequences, consequences that limit the use of vectors whose integration is random, e.g., retroviruses. The Rep protein mediates the integration of the genetic construct containing the AAV ITRs and the transgene. The use of AAV is advantageous for its predictable integration site and because it has not been associated with human or non-human primate diseases, thus obviating many of the concerns that have been raised with virus-derived gene therapy vectors.

“Portion of the genetic construct integrates into a chromosome” refers to the portion of the genetic construct that will become covalently bound to the genome of the cell upon introduction of the genetic construct into the cell via administration of the gene therapy particle. The integration is mediated by the AAV ITRs flanking the transgene and the AAV Rep protein. Portions of the genetic construct that may be integrated into the genome include the transgene and the AAV ITRs.

The “transgene” may contain a transgenic sequence or a native or wild-type DNA sequence. The transgene may become part of the genome of the primate subject. A transgenic sequence can be partly or entirely species-heterologous, i.e., the transgenic sequence, or a portion thereof, can be from a species which is different from the cell into which it is introduced.

As used herein, the term “stably maintained” refers to characteristics of transgenic non-human primates that maintain at least one of their transgenic elements (i.e., the element that is desired) through multiple generations of cells. For example, it is intended that the term encompass many cell division cycles of the originally transfected cell. The term “stable transfection” or “stably transfected” refers to the introduction and integration of foreign DNA into the genome of the cell. The term “stable transfectant” refers to a cell that has stably integrated foreign DNA into the genomic DNA.

As used herein, the terms “transgene encoding,” “nucleic acid molecule encoding,” “DNA sequence encoding,” and “DNA encoding” refer to the order or sequence of deoxyribonucleotides along a strand of deoxyribonucleic acid. The order of these deoxyribonucleotides may, for example, determine the order of amino acids along the polypeptide (protein) chain. The DNA sequence thus may code for the amino acid sequence.

As used herein, the term “wild type” (wt) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants may be isolated, which are identified by the acquisition of altered characteristics when compared to the wild-type gene or gene product.

As used herein, the term “AAV virion,” “AAV particle,” or “AAV gene therapy particle,” “AAV gene therapy vector,” or “rAAV gene therapy vector” refers to a complete virus unit, such as a wt AAV virus particle (comprising a linear, single-stranded AAV nucleic acid genome associated with at least one AAV capsid protein coat). In this regard, single-stranded AAV nucleic acid molecules of either complementary sense (e.g., “sense” or “antisense” strands) can be packaged into any one AAV virion and both strands are equally infectious. Also included are infectious viral particles containing a heterologous DNA molecule of interest (e.g., CFTR or a biologically active portion thereof), which is flanked on both sides by AAV ITRs.

As used herein, the term “transfection” refers to the uptake of foreign DNA by a cell. A cell has been “transfected” when exogenous DNA has been introduced inside the cell membrane. A number of transfection techniques are generally known in the art (See e.g., Graham et al., Virol., 52:456 (1973); Sambrook et al., Molecular Cloning, a Laboratory Manual, Cold Spring Harbor Laboratories, New York (1989); Davis et al., Basic Methods in Molecular Biology, Elsevier, (1986); and Chu et al., Gene 13:197 (1981). Such techniques may be used to introduce one or more exogenous DNA moieties, such as a gene transfer vector and other nucleic acid molecules, into suitable recipient cells.

As used herein, the terms “stable transfection” and “stably transfected” refers to the introduction and integration of foreign DNA into the genome of the transfected cell. The term “stable transfectant” refers to a cell, which has stably integrated foreign DNA into the genomic DNA.

As used herein, the term “transient transfection” or “transiently transfected” refers to the introduction of foreign DNA into a cell wherein the foreign DNA fails to integrate into the genome of the transfected cell and is maintained as an episome. During this time the foreign DNA is subject to the regulatory controls that govern the expression of endogenous genes in the chromosomes. The term “transient transfectant” refers to cells which have taken up foreign DNA but have failed to integrate this DNA. As used herein, the term “transduction” denotes the delivery of a DNA molecule to a recipient cell either in vivo or in vitro, via a replication-defective viral vector, such as via a recombinant AAV virion.

As used herein, the term “recipient cell” refers to a cell which has been transfected or transduced, or is capable of being transfected or transduced, by a nucleic acid construct or vector bearing a selected nucleotide sequence of interest. The term includes the progeny of the parent cell, whether or not the progeny are identical in morphology or in genetic make-up to the original parent, so long as the selected nucleotide sequence is present. The recipient cell may be the cells of a subject to which the gene therapy particles and/or gene therapy vector has been administered.

As used herein, the term “nucleic acid” sequence refers to a DNA or RNA sequence. Nucleic acids can, for example, be single or double stranded. The term includes sequences such as any of the known base analogues of DNA and RNA.

As used herein, the term “recombinant DNA molecule” refers to a DNA molecule which is comprised of segments of DNA joined together by means of molecular biological techniques.

As used herein, the term “regulatory element” refers to a genetic element which can control the expression of nucleic acid sequences. For example, a promoter is a regulatory element that facilitates the initiation of transcription of an operably linked coding region. Other regulatory elements are splicing signals, polyadenylation signals, termination signals, etc.

The term DNA “control sequences” refers collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need be present.

Transcriptional control signals in eukaryotes generally comprise “promoter” and “enhancer” elements. Promoters and enhancers consist of short arrays of DNA sequences that interact specifically with cellular proteins involved in transcription (Maniatis et al., Science 236:1237 (1987)). Promoter and enhancer elements have been isolated from a variety of eukaryotic sources including genes in yeast, insect and mammalian cells and viruses (analogous control sequences, i.e., promoters, are also found in prokaryotes). The selection of a particular promoter and enhancer depends on the recipient cell type. Some eukaryotic promoters and enhancers have a broad host range while others are functional in a limited subset of cell types (See e.g., Voss et al., Trends Biochem. Sci., 11:287 (1986); and Maniatis et al., supra, for reviews). For example, the SV40 early gene enhancer is very active in a variety of cell types from many mammalian species and has been used to express proteins in a broad range of mammalian cells (Dijkema et al, EMBO J. 4:761 (1985)). Promoter and enhancer elements derived from the human elongation factor 1-alpha gene (Uetsuki et al., J. Biol. Chem., 264:5791 (1989); Kim et al., Gene 91:217 (1990); and Mizushima and Nagata, Nucl. Acids. Res., 18:5322 (1990)), the long terminal repeats of the Rous sarcoma virus (Gorman et al., Proc. Natl. Acad. Sci. U.S.A. 79:6777 (1982)), and the human cytomegalovirus (Boshart et al., Cell 41:521 (1985)) are also of utility for expression of proteins in diverse mammalian cell types. Promoters and enhancers can be found naturally, alone or together. For example, retroviral long terminal repeats comprise both promoter and enhancer elements. Generally promoters and enhancers act independently of the gene being transcribed or translated. Thus, the enhancer and promoter used can be “endogenous,” “exogenous,” or “heterologous” with respect to the gene to which they are operably linked. An “endogenous” enhancer/promoter is one which is naturally linked with a given gene in the genome. An “exogenous” or “heterologous” enhancer and promoter is one which is placed in juxtaposition to a gene by means of genetic manipulation (i.e., molecular biological techniques) such that transcription of that gene is directed by the linked enhancer/promoter.

As used herein, the term “tissue specific” refers to regulatory elements or control sequences, such as a promoter, enhancers, etc., wherein the expression of the nucleic acid sequence is substantially greater in a specific cell type(s) or tissue(s). In particularly preferred embodiments, the CB promoter (CB is the same as CBA defined above) displays good expression of human CFTR, rAAV5-CB-.DELTA.264CFTR, rAAV5-CB-.DELTA.27-264CFTR, or another biologically active portion of CFTR. It is not intended, however, that the present invention be limited to the CB promoter or to lung specific expression, as other tissue specific regulatory elements, or regulatory elements that display altered gene expression patterns, are encompassed within the invention.

The presence of “splicing signals” on: an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York (1989), pp. 16.7-16.8). A commonly used splice donor and acceptor site is the splice junction from the 16S RNA of SV40.

Transcription termination signals are generally found downstream of a polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which has been isolated from one gene and operably linked to the 3′ end of another gene. A commonly used heterologous poly A signal is the SV40 poly A signal. The SV40 poly A signal is contained on a 237 bp BamHI/BclI restriction fragment and directs both termination and polyadenylation (Sambrook et al., supra, at 16.6-16.7).

The terms “operably linked” and “operatively linked” refer to the regulatory sequences for expression of the coding sequence that are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

As used herein, the term “subject” refers to humans and other primates.

As defined herein, a “therapeutically effective amount” or “therapeutic effective dose” is an amount or dose of AAV particles or virions capable of producing sufficient amounts of a desired protein to restore the activity of the protein, thus providing a palliative tool for clinical intervention. A therapeutically effective amount or dose of transfected AAV particles that confer expression of a TREE (e.g., LEN), for example, to a patient will effect tissue regeneration.

The results presented herein demonstrate that:
1. Upon injury, the zebrafish Leptin homolog lepb is sharply induced in regenerating fins and hearts.
2. A short DNA element 7 kb upstream to lepb start colon is marked by acetylated lysine 27 of Histone H3 (H3K27ac), an indicator of active enhancers.
3. This lepb-linked regulatory enhancer element (LEN), a 1350 bp DNA sequence, can direct expression of a transgenic reporter in vertebrate species in injury and/or regeneration contexts, in combination with minimal promoters.
4. The LEN element is separable into tissue-specific regulatory modules. LEN fragments composed of nucleotides 663-854 and 1034-1350 can direct reporter expression in regenerating fin and heart tissue, respectively.
5. The engineering of TREEs such as LEN when paired with pro- or anti-regenerative factors, can be applied to modulate the regenerative capacities of fin and heart tissue in zebrafish.

These results are the first to (1) identify and validate the activity of tissue regeneration elements; and (2) demonstrate that enhancer element engineering can be used as a means to modulate tissue repair.

Accordingly, one aspect of the present disclosure provides a gene therapy construct comprising, consisting of, or consisting essentially of a nucleic acid encoding one or more tissue regeneration enhancer elements (TREEs) operatively linked to a promoter. In some embodiments, the gene therapy construct further comprises a nucleic acid sequence comprising one or more pro- or anti-regenerative factors, the expression of which is under the influence of the tissue regeneration enhancer elements.

In other embodiments, the gene therapy particle comprises a vector system. In some embodiments, the vector system comprises an AAV vector system.

In some embodiments, the gene therapy construct further comprises a first and second AAV inverted terminal repeat (ITR) sequence flanking the sequence encoding the TREEs. In certain embodiments, the ITR nucleotide sequences are derived from AAV serotype 2 (AAV-2).

Another aspect of the present disclosure provides a pharmaceutical composition comprising the gene therapy construct provided herein in a biocompatible pharmaceutical carrier.

In another embodiment, the TREE comprises the lepb-linked regulatory enhancer element (LEN).

One aspect of the present disclosure provides a method of treating or ameliorating tissue repair comprising, consisting of, or consisting essentially of administering to a subject a therapeutically effective amount of gene therapy construct as provided herein such that the tissue repair is treated or ameliorated.

Another aspect of the present disclosure provides a method of augmenting tissue regeneration in a subject comprising, consisting of, or consisting essentially of administering to the subject a therapeutically effective amount of a gene therapy construct as described herein such that the tissue regeneration is augmented.

Another aspect of the present disclosure provides a method of delivering cell therapy to a subject comprising, consisting of, or consisting essentially of inserting within the genome of reprogrammed stem/progenitor cells of the subject a gene therapy construct as provided herein such that cell therapy is delivered.

Another aspect of the present disclosure provides a method for screening drugs that modulate regenerative capacity comprising, consisting of, or consisting essentially of transfecting a gene construct as described herein into a model system, administering to the system a drug of interest, and measuring regenerative capacity in response to the drug.

In some embodiments, the model system comprises a zebrafish model system.

Another aspect of the present disclosure provides all that is disclosed and illustrated herein.

### EXAMPLES

The following examples are provided as illustration and not by way of limitation.

### General Methods

### Zebrafish Maintenance and Procedures

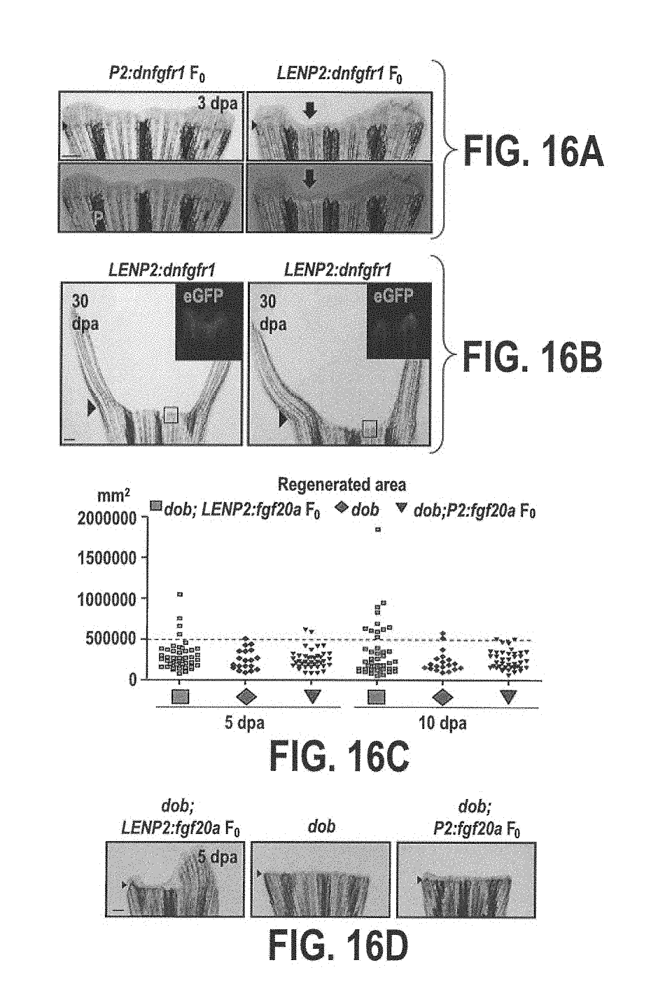

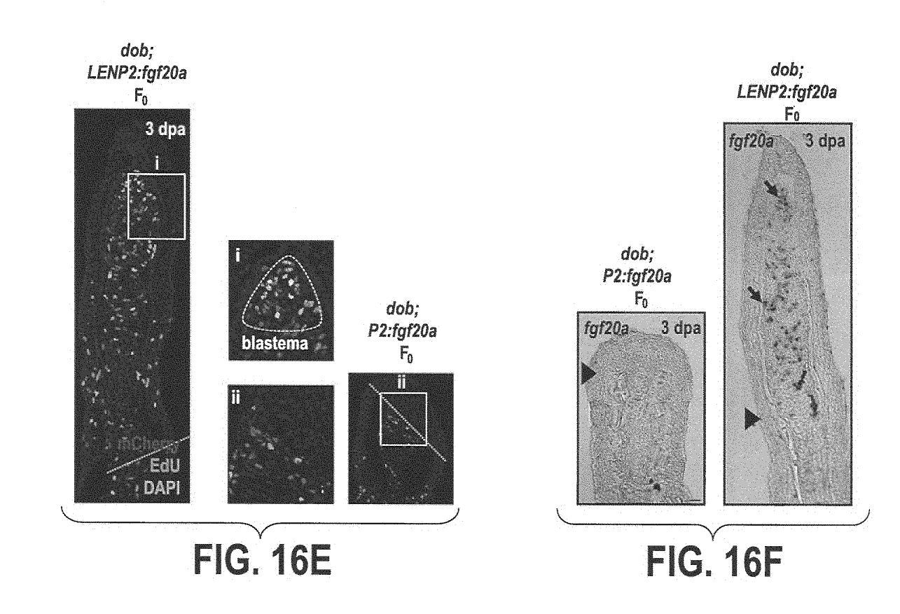

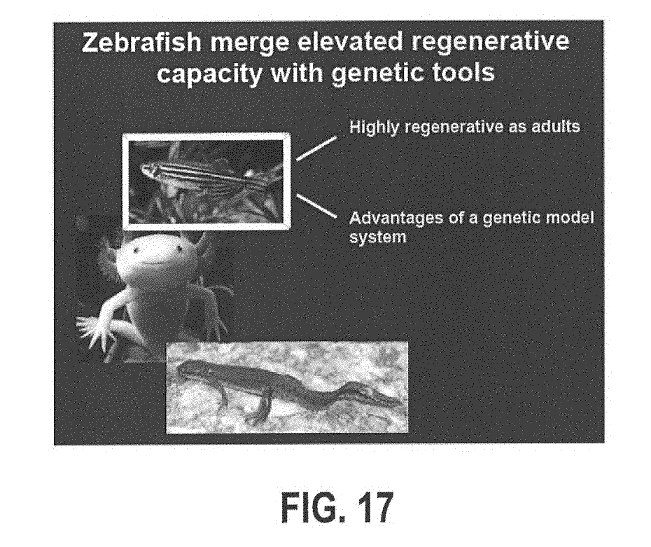

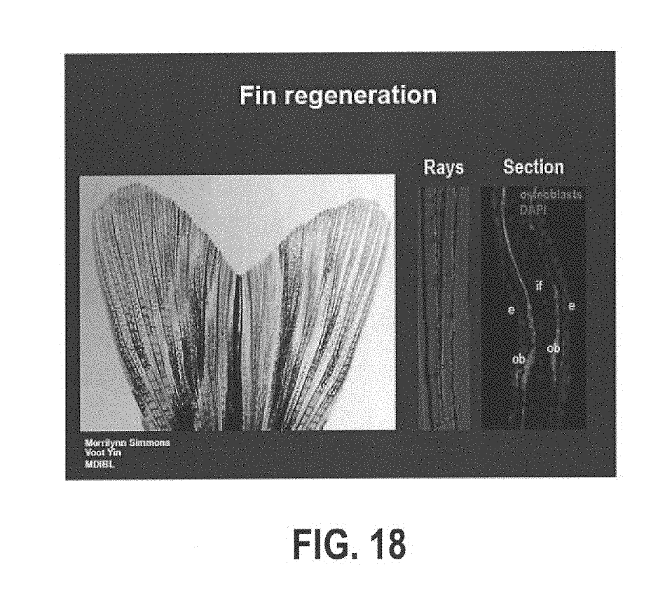

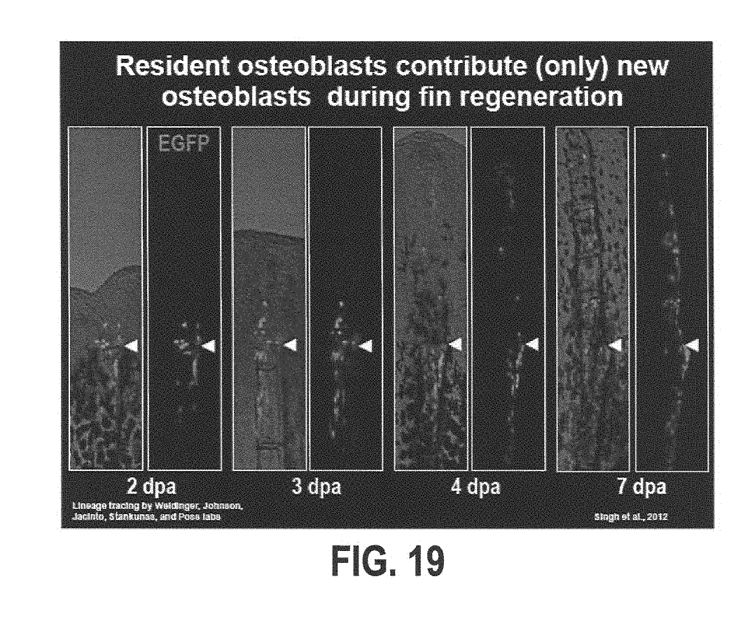

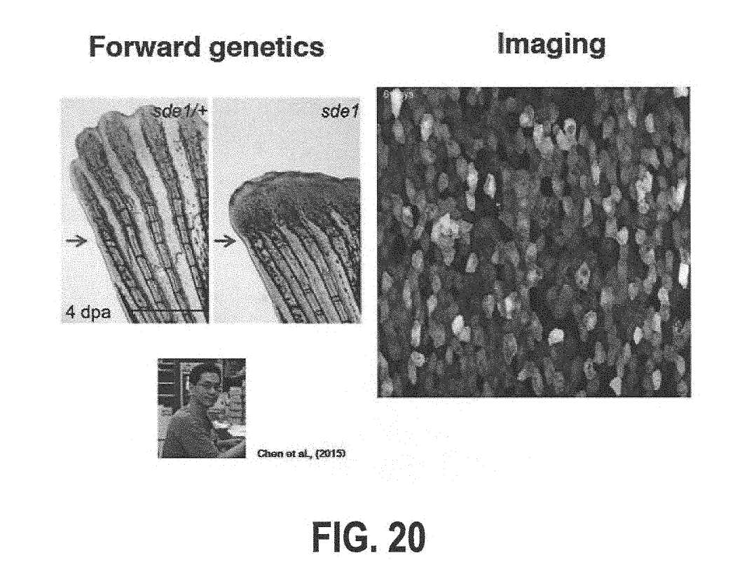

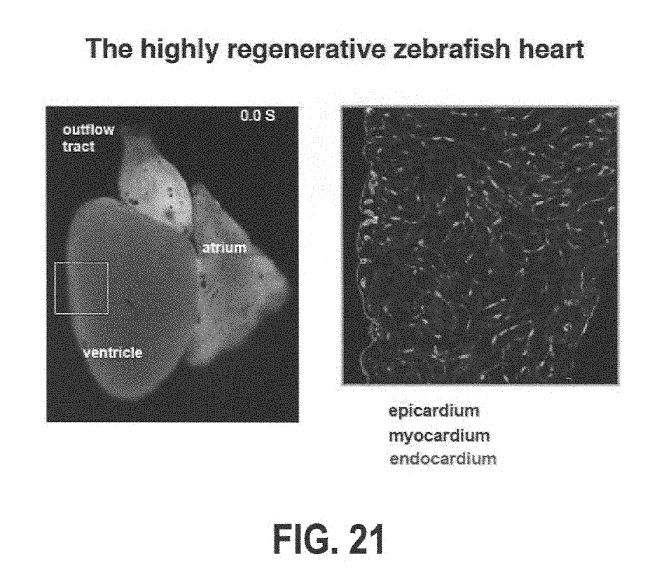

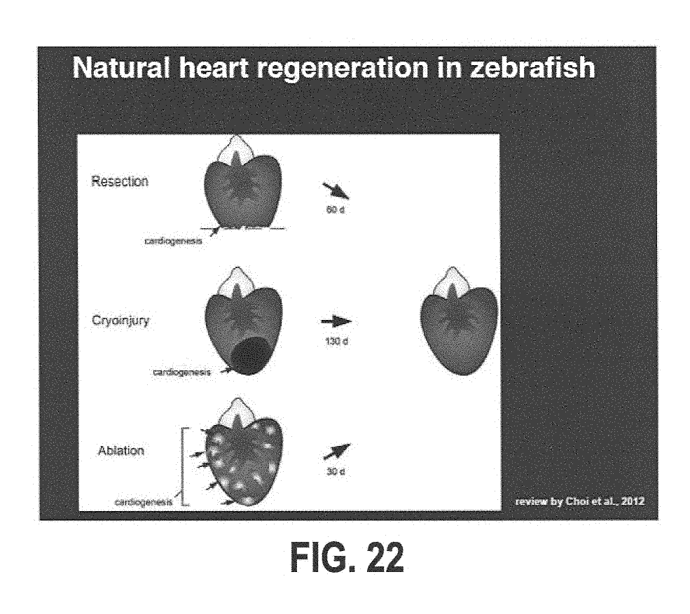

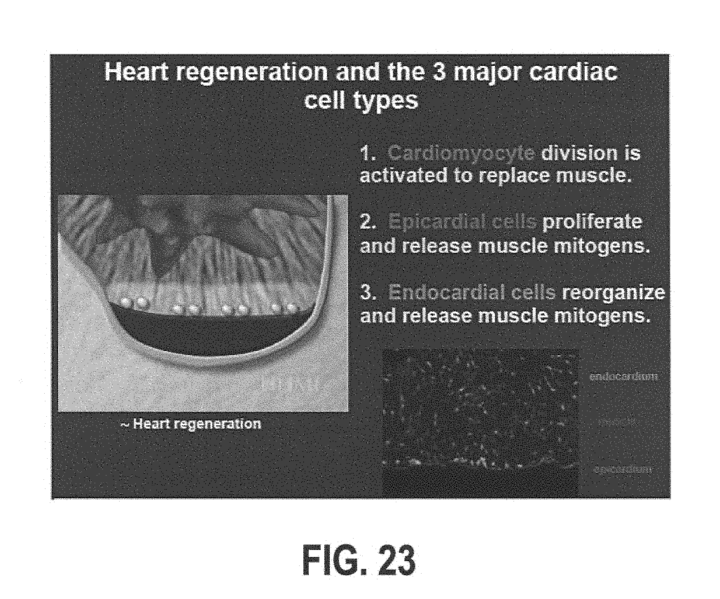

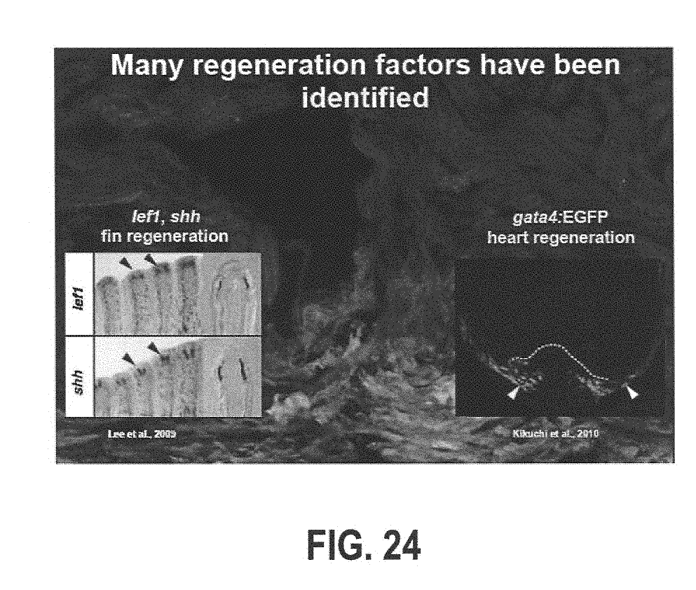

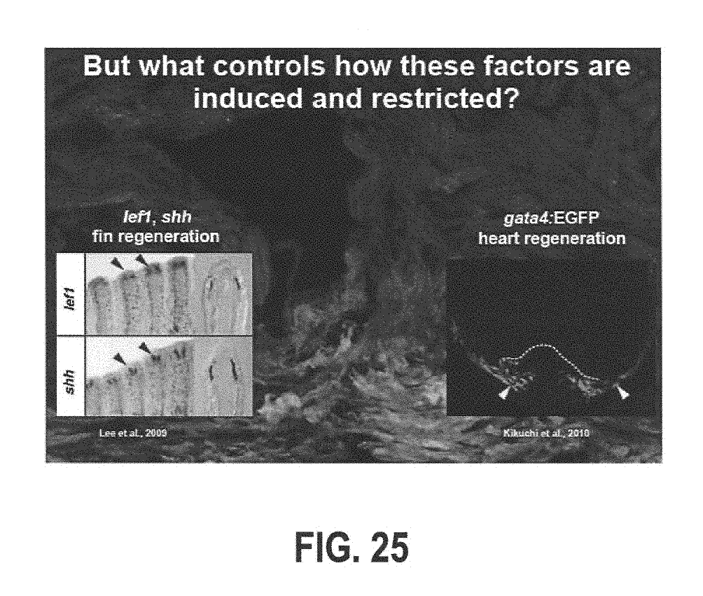

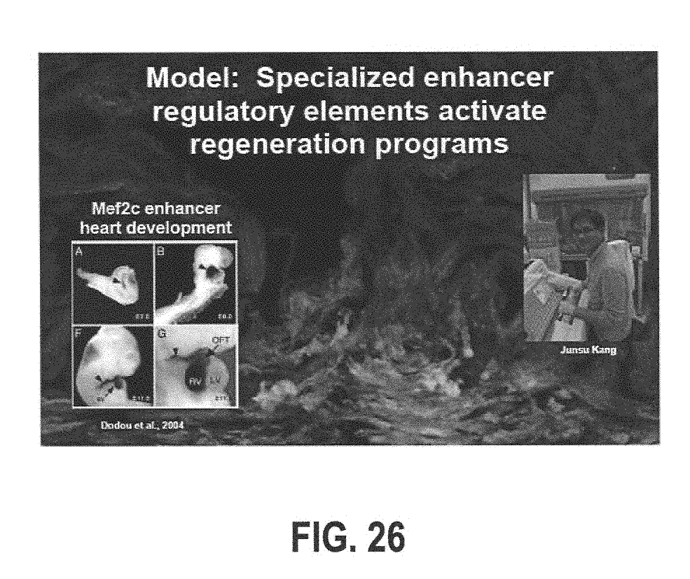

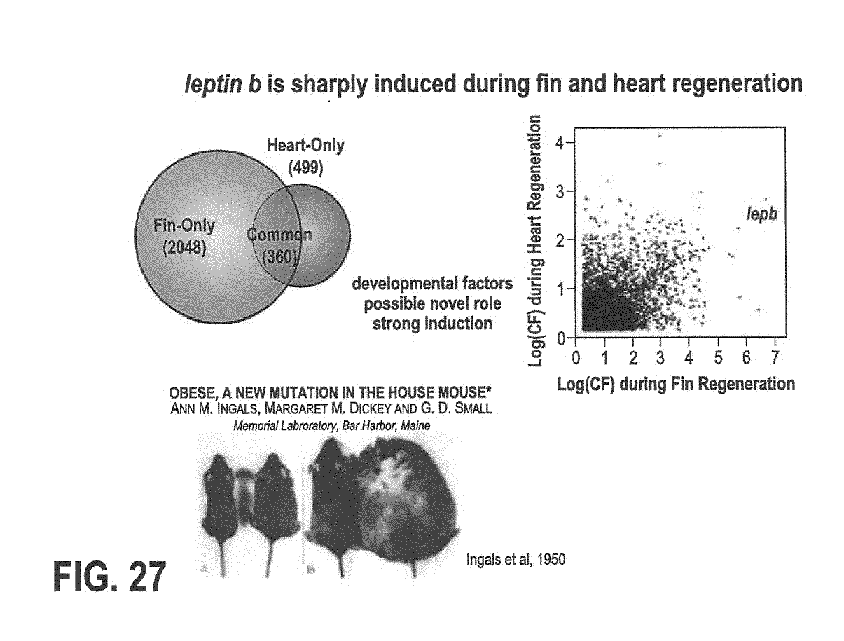

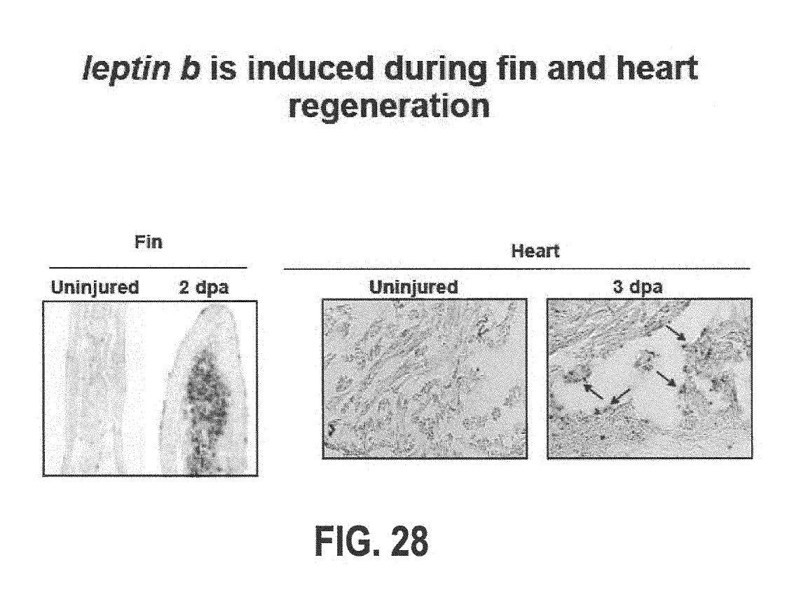

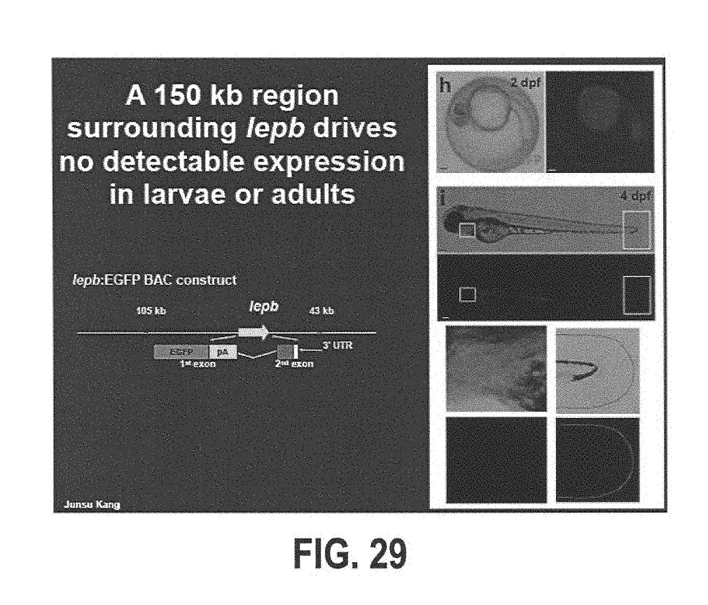

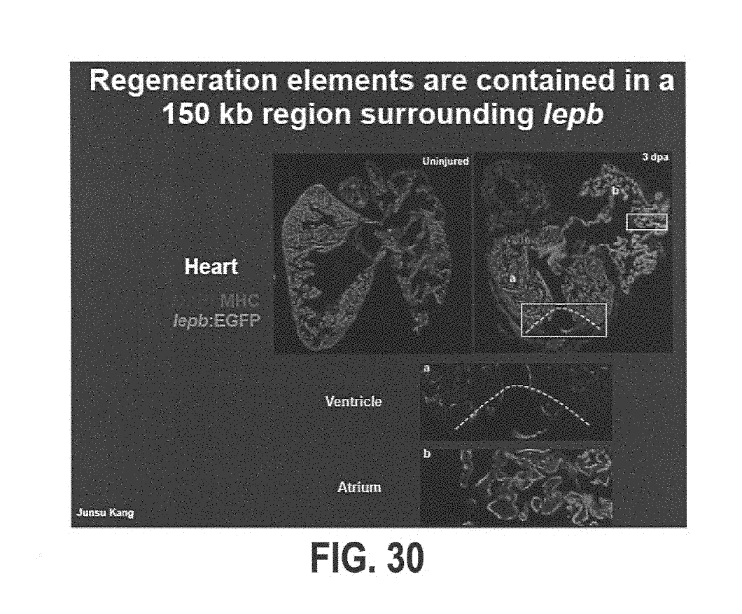

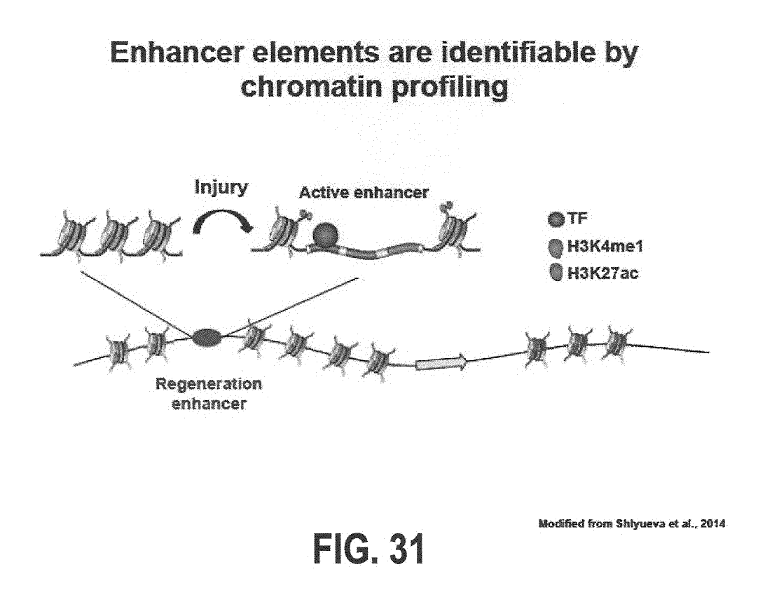

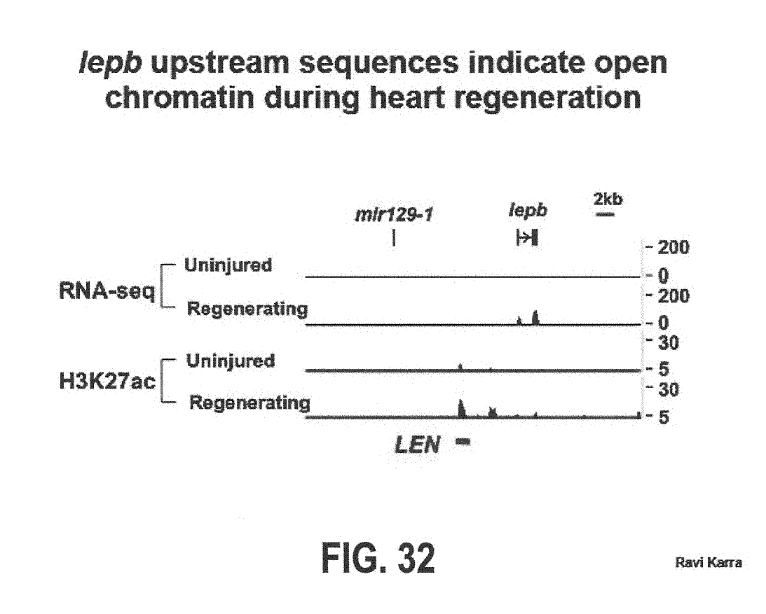

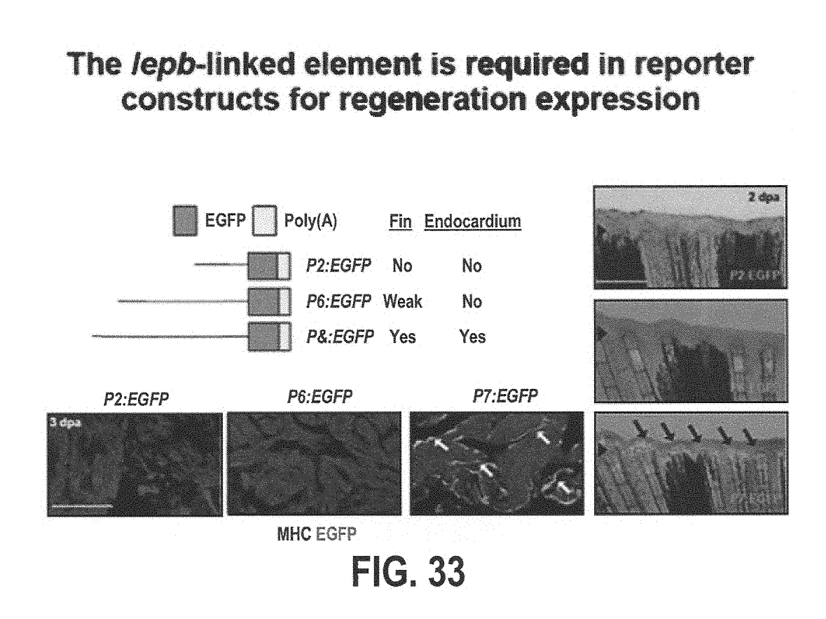

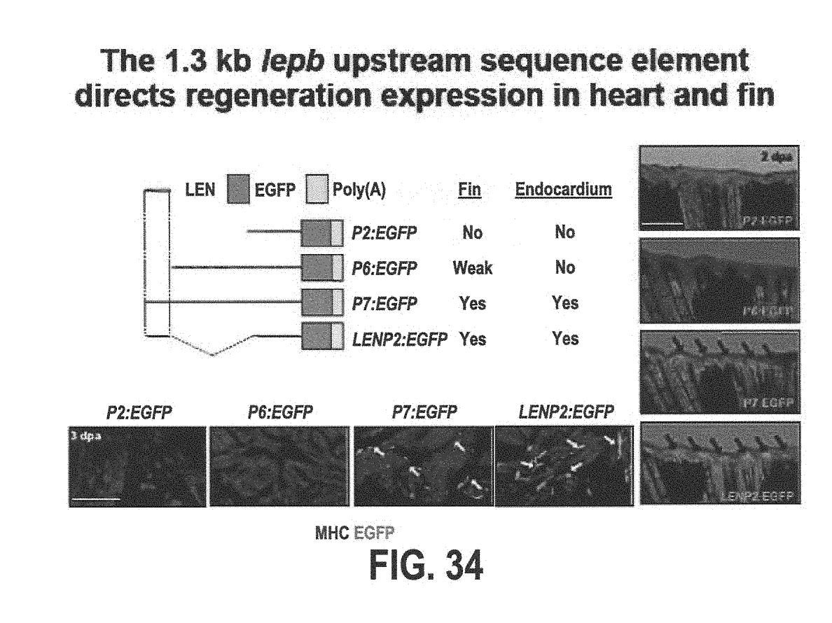

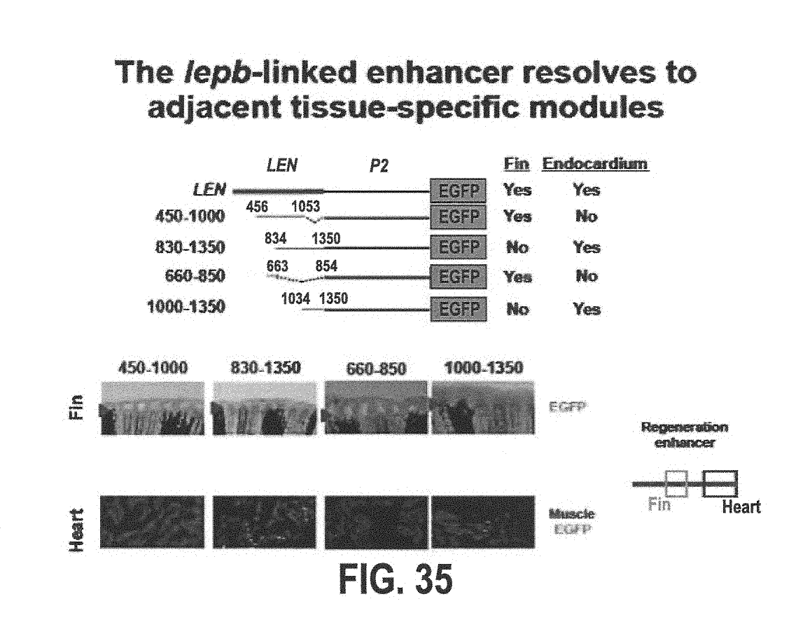

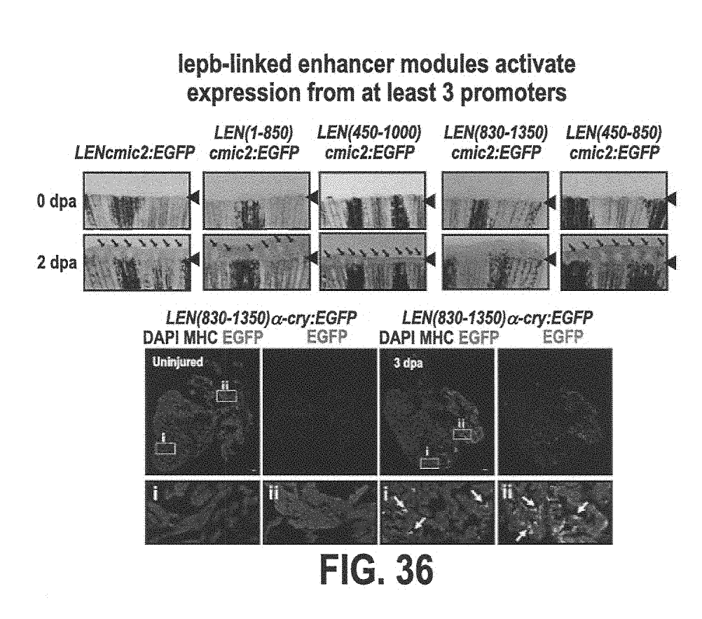

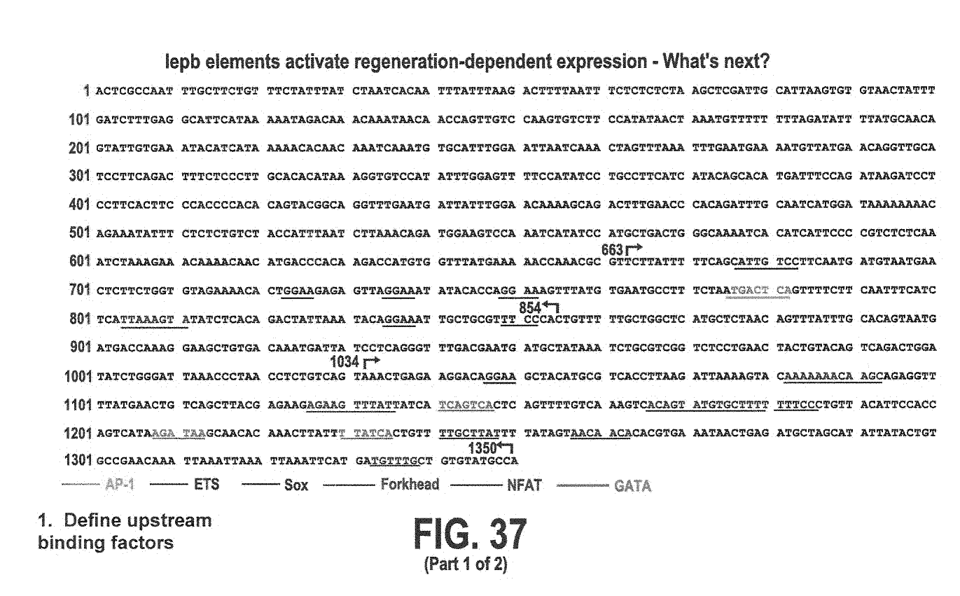

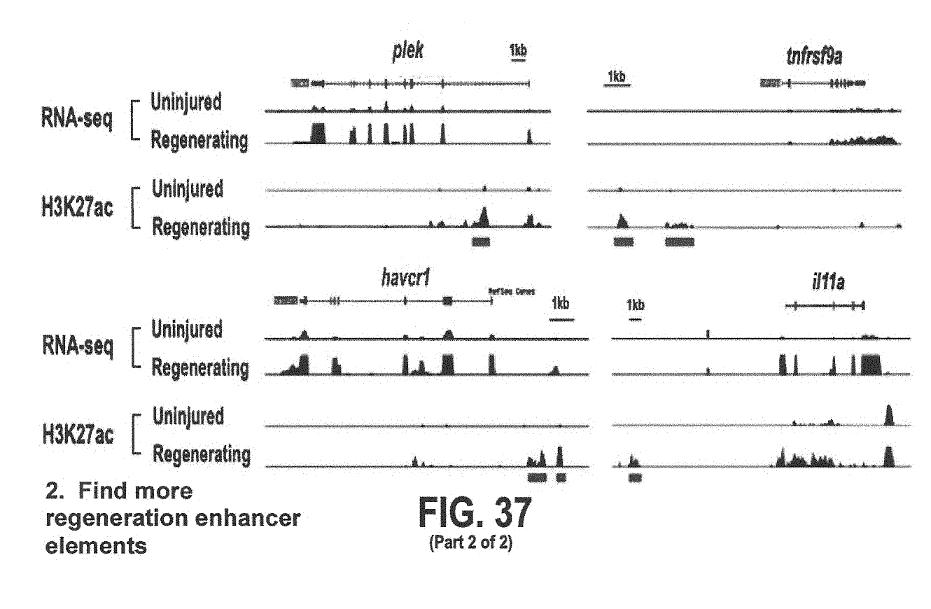

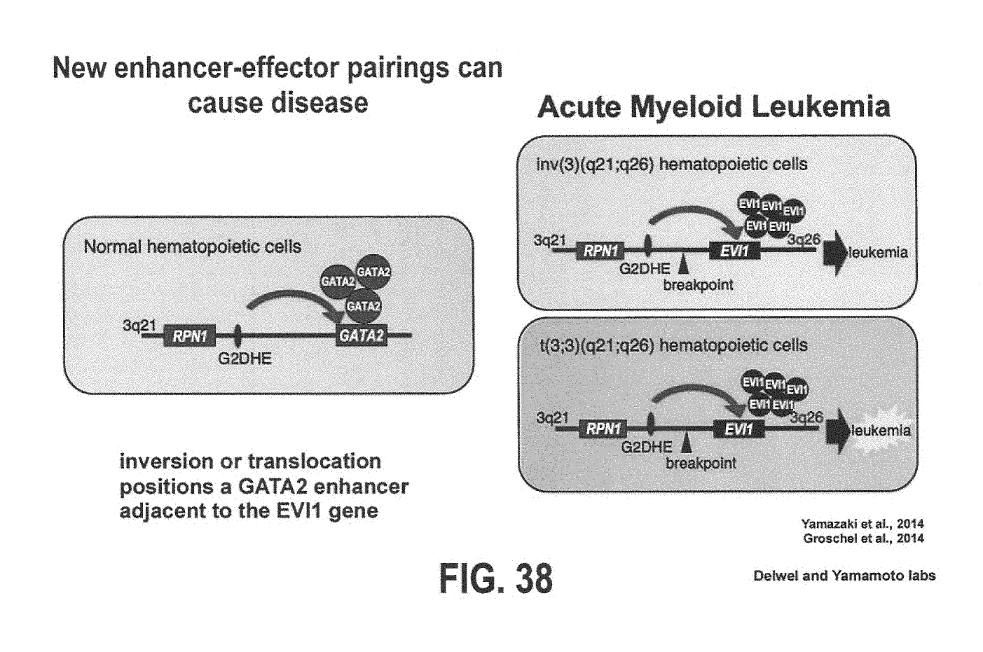

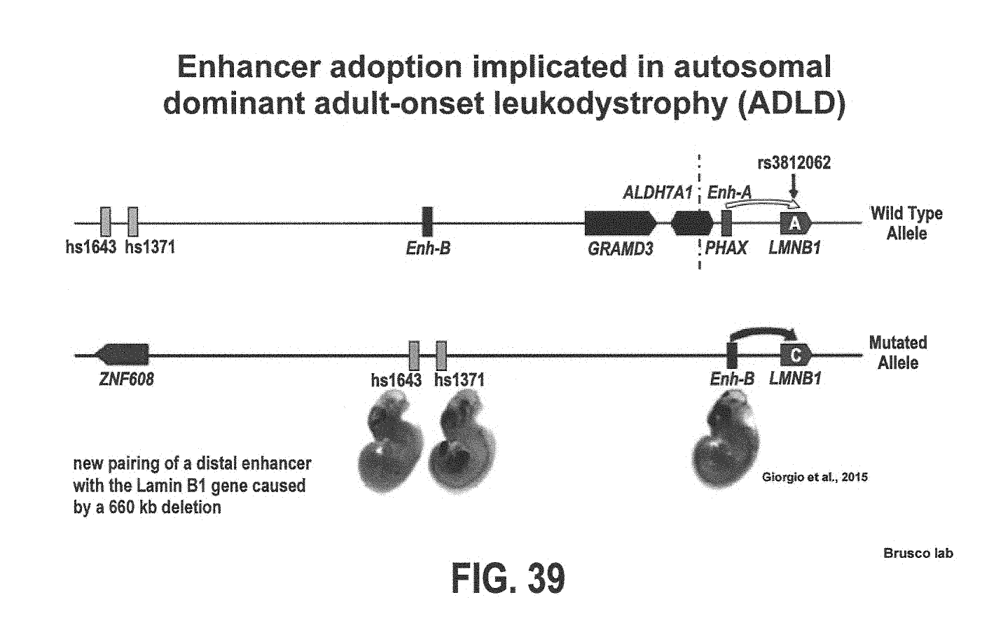

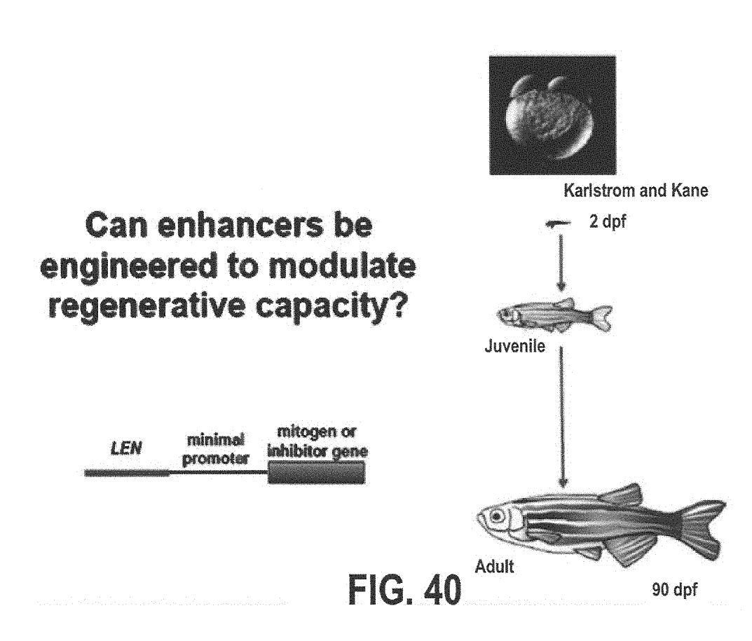

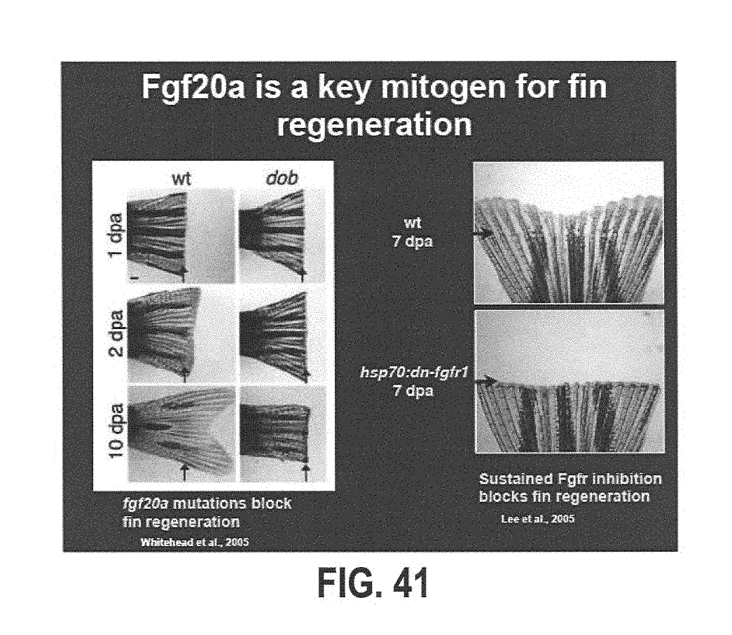

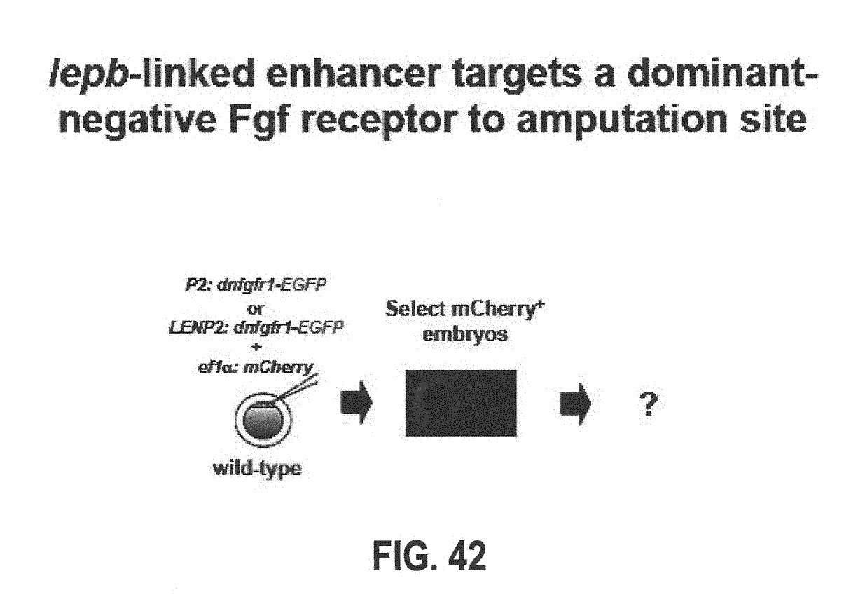

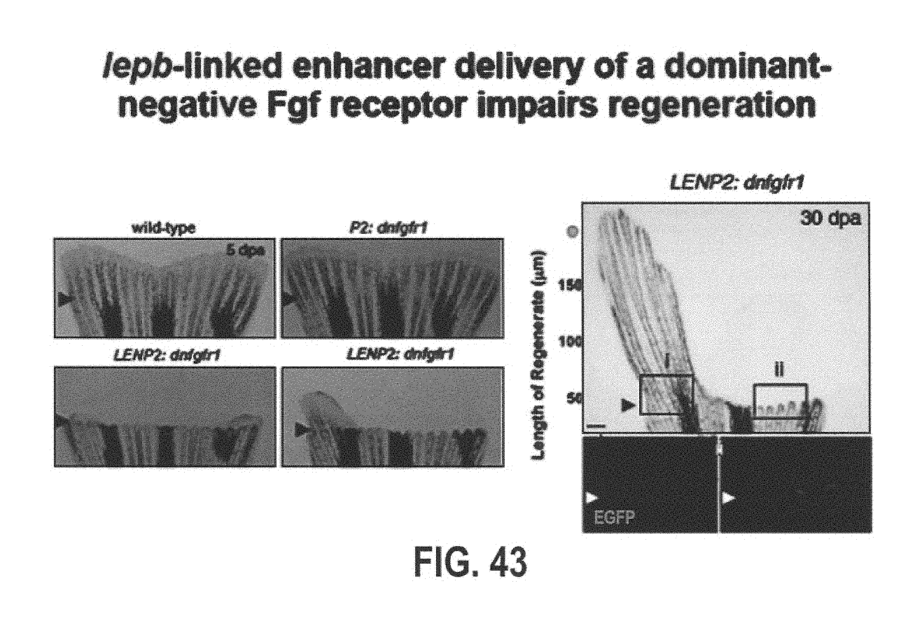

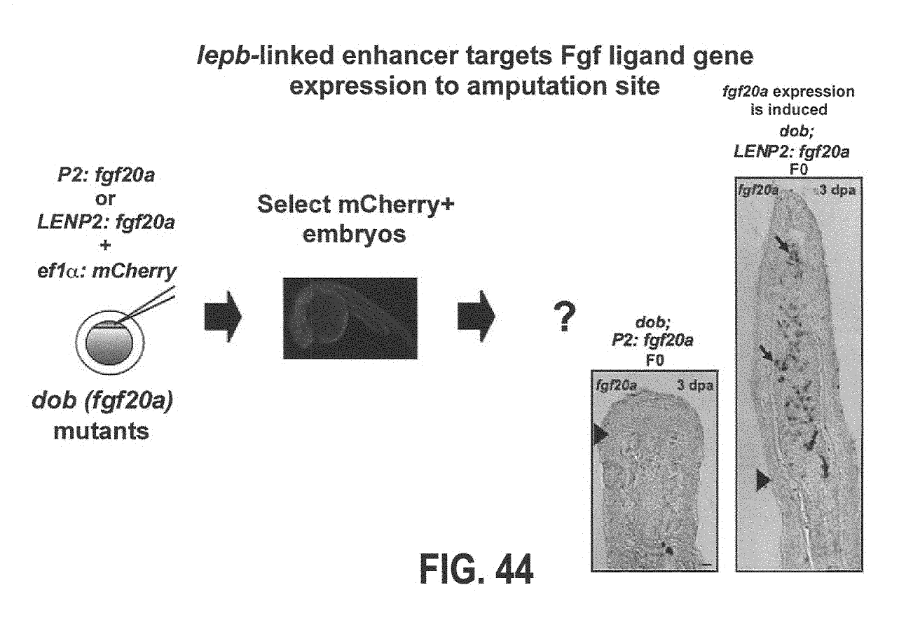

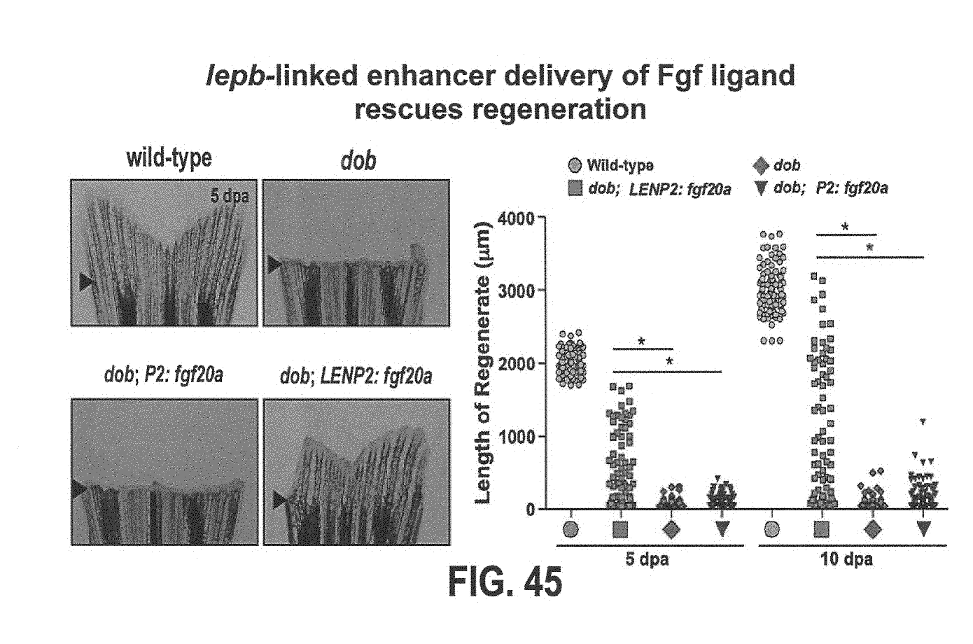

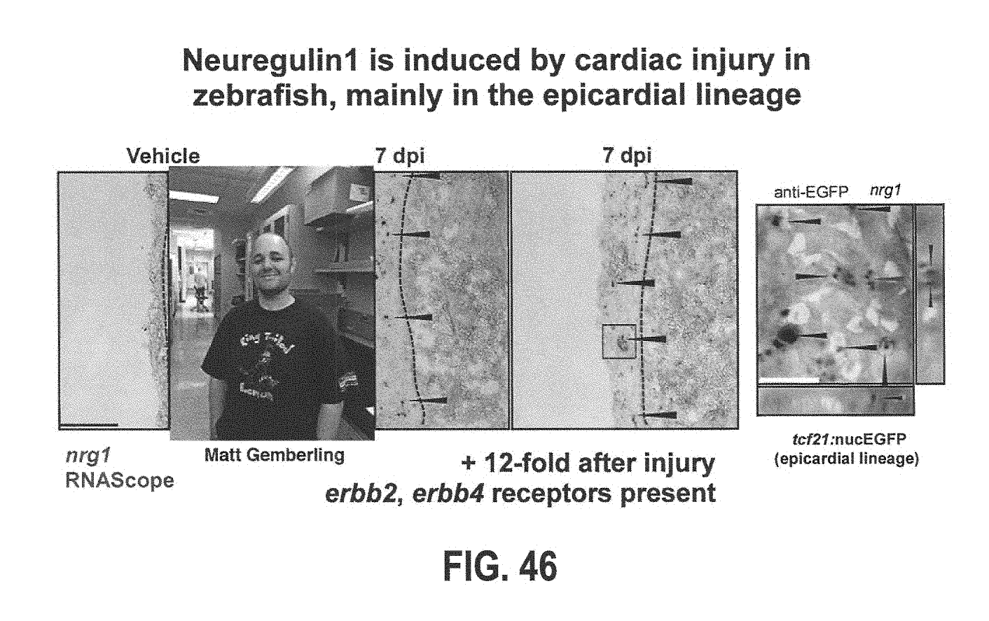

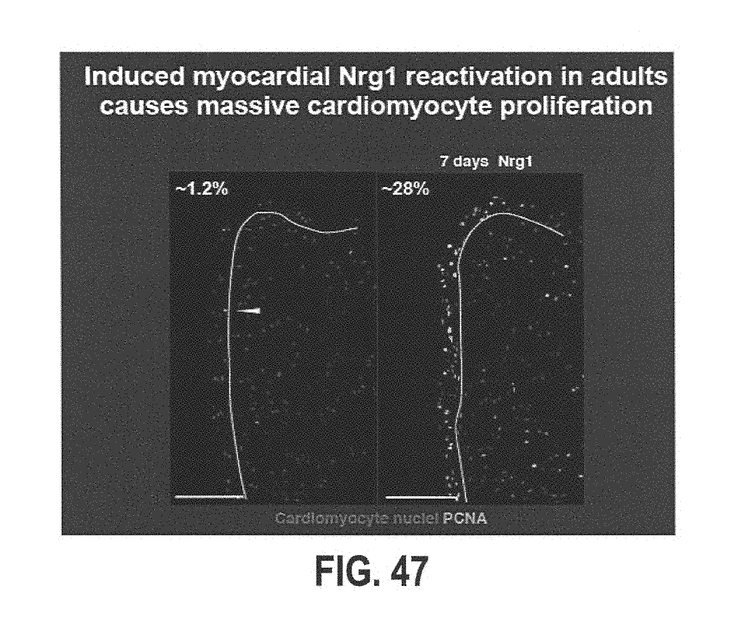

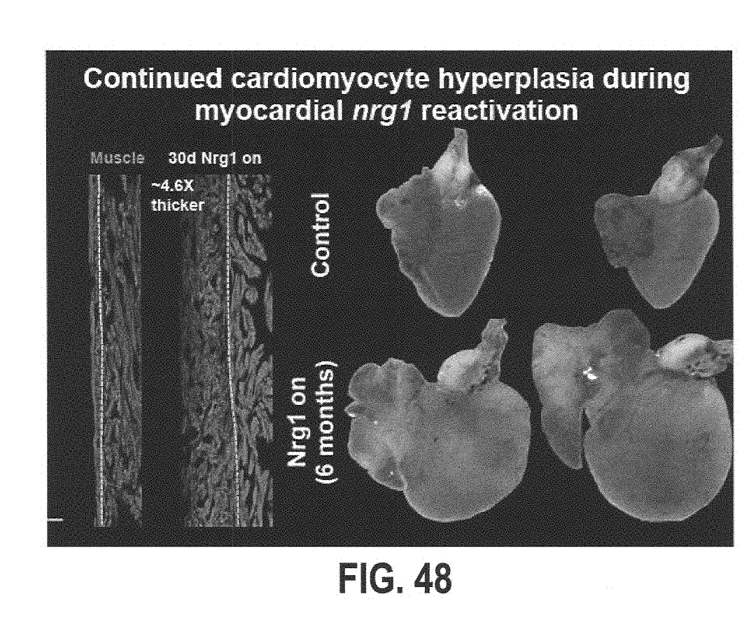

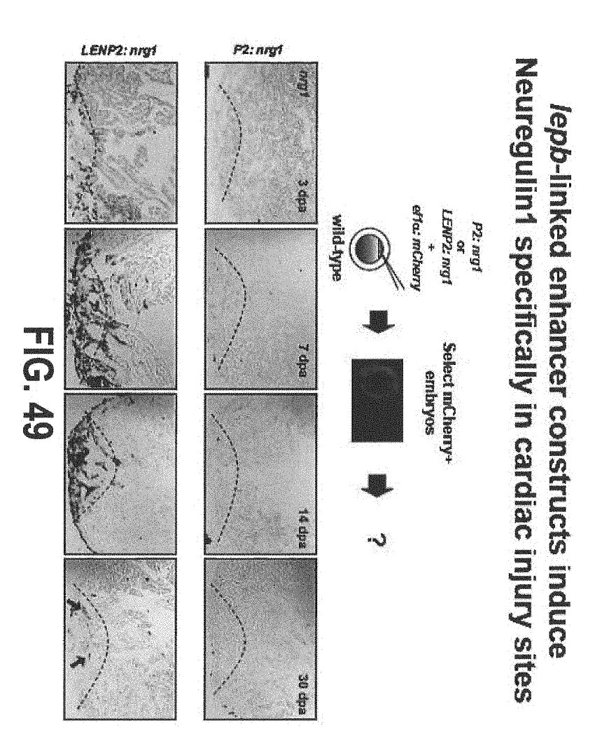

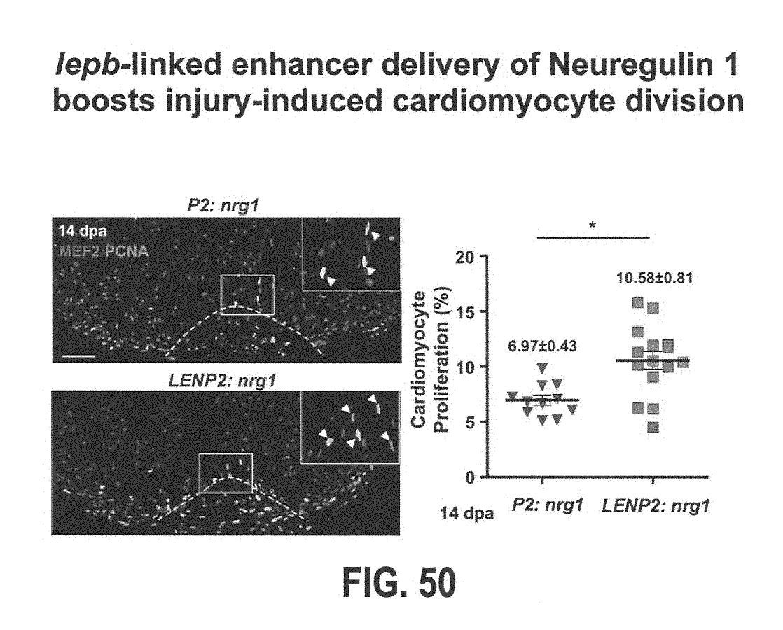

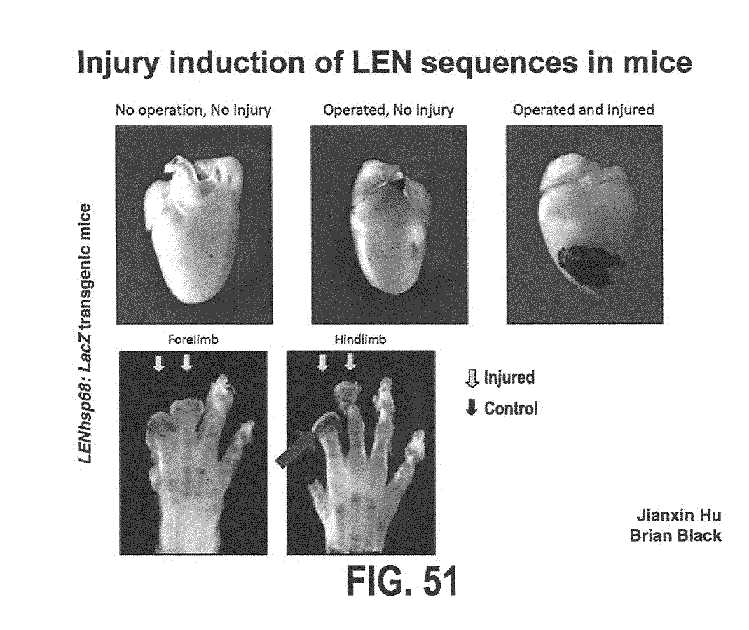

Wild-type or transgenic male and female zebrafish of the outbred Ekkwill (EK) strain were used for all experiments, with adults ranging in age from 3 to 12 months. Water temperature was maintained at 26° C. for animals unless otherwise indicated. Fins were amputated to 50% of their original length using razor blades. As penetrance of the dob mutation was higher at 33° C. than at 26° C., dob fish were maintained at 33° C. after caudal fin amputation. To measure lengths of regenerates, lengths from the amputation plane to the distal tips of the 3^{rd} and 4^{th} fin rays of dorsal and ventral caudal fin lobes were determined using ZEN software. Because some dob animals regenerated portions of the 1^{st} and 2^{nd} fin rays of ventral lobes, regenerating caudal fin areas for FIG. 16C were measured from the dorsal 3^{rd} fin ray to the ventral 3^{rd} fin ray and calculated using ZEN software. Partial ventricular resection surgeries were performed as described previously³⁹, in which ˜20% of the cardiac ventricle was removed at the apex. To ablate cardiomyocytes, cmlc2:CreER; bactin2loxp-mCherry-STOP-loxp-DTA (Z-CAT) fish were used⁴⁰. Z-CAT zebrafish were incubated in vehicle (0.01% EtOH) or 10 μM tamoxifen for 12 hours. Work with zebrafish was performed in accordance with Duke University guidelines.

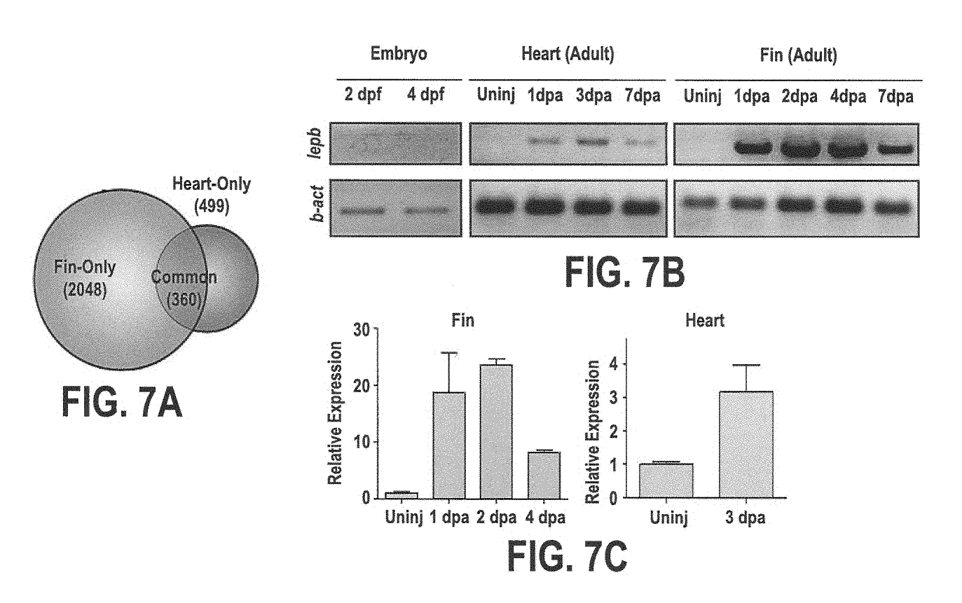

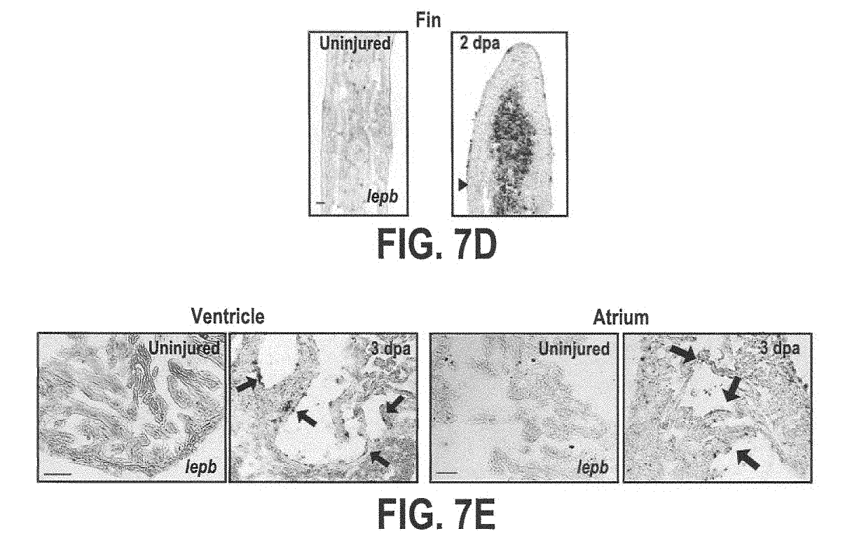

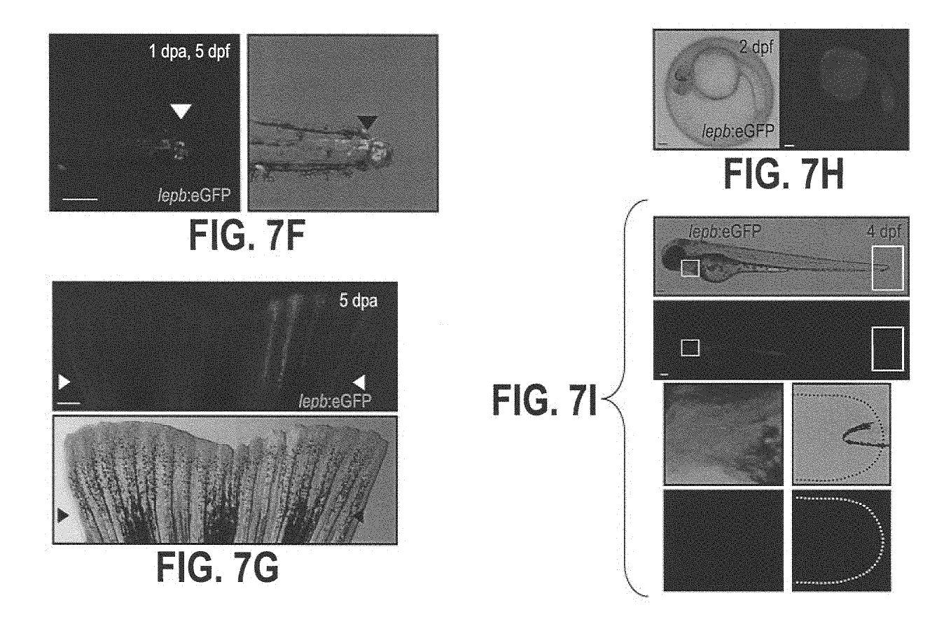

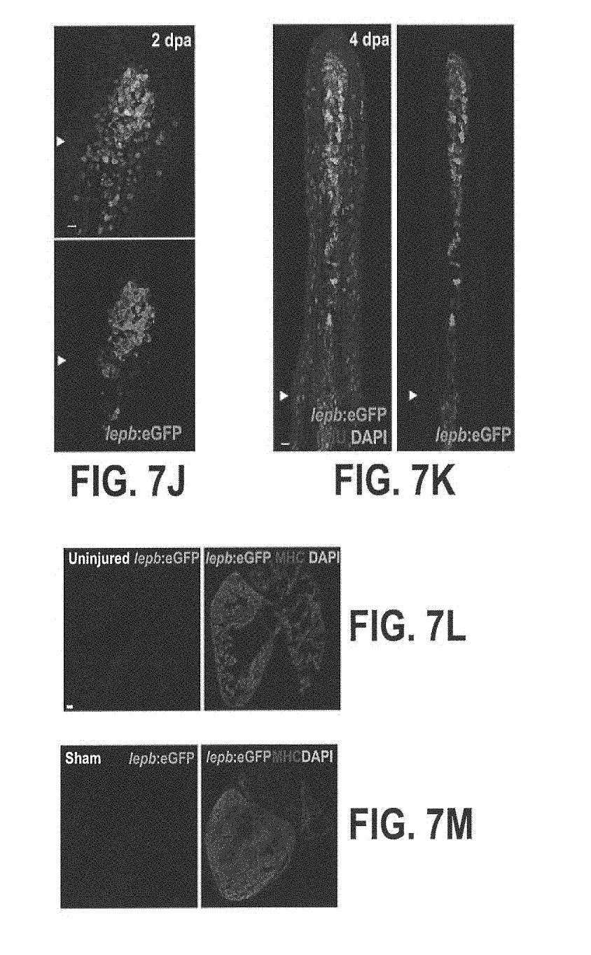

To generate lepb:eGFP BAC transgenic animals (full names, Tg(lepb:eGFP)^{pd120} and Tg(lepb:eGFP)^{pd121}), the iTol2 cassette⁴¹ was integrated into the BAC clone DKEY-21022 using Red/ET recombineering technology (GeneBridges). Then, the first exon of the lepb gene in the BAC clone DKEY-21022 was replaced with an eGFP cassette by Red/ET recombineering. 5′ and 3′ homology arms were amplified by PCR (Supplementary Information) and subcloned into the pCS2-eGFP plasmid. One nl of 50 ng/μl purified, recombined BAC was injected into one-cell stage zebrafish embryos along with one nl of 30 ng/μl synthetic Tol2 mRNA⁴¹. To sort F₀ transgenic animals injected with lepb:eGFP constructs, fin folds were amputated at 3 or 4 dpf, and embryos displaying eGFP fluorescence near the injury site at 1 dpa were selected, (FIG. 7F). After raising F₀ zebrafish to adulthood, caudal fins were amputated and zebrafish displaying induced eGFP were selected for breeding (FIG. 7G). Between 30-60 dpf, caudal fins of progeny from transgene-positive F₀ fish were amputated, and eGFP⁺ transgenic animals were isolated to identify stable lines. Two lines were identified that had indistinguishable expression features.

To define LEN activity, over 60 additional new transgenic lines were established in this study, listed in Table 1. To generate transgenic animals, DNA sequences were amplified by PCR with indicated primers (Table 2) and subcloned into a pCS2-eGFP-1-scel vector, in which 1-Scel restriction sites were flanked by a multiple cloning site. As promoters, 2 kb, 1.6 kb, and 0.7 kb upstream sequences of lepb, cmlc2⁴² and α-cry⁴³ genes were used, respectively. These constructs were injected into one-cell-stage wild-type or dob embryos using standard meganuclease transgenesis techniques. 2 kb lepb upstream sequences could induce transgene expression after fin fold amputation at larval stages, but never alter caudal fin amputation in adults. To isolate stable lines, larvae were examined for transgene expression near injury site in response to fin fold amputation (2 kb lepb), in cardiomyocytes (1.6 kb cmlc2), and in lens (0.7 kb α-cry).

TABLE 1
List of transgenic zebrafish lines.

| Strain Name | Allele |
|---|---|
| lepb | pd94 |
| Tg(lepr:lepr-mCherry BAC) | pd95 |
| Tg(lepb:EGFP BAC) line 2 | pd120 |
| Tg(lepb:EGFP BAC) line 13 | pd121 |
| Tg(lepb up 2kb:EGFP) line 2 | pd122 |
| Tg(lepb up 2kb:EGFP) line 3 | pd123 |
| Tg(lepb up 2kb:EGFP) line 14 | pd124 |
| Tg(lepb up 6kb:EGFP) line 1 | pd125 |
| Tg(lepb up 6kb:EGFP) line 10 | pd126 |
| Tg(lepb up 7kb:EGFP) line 3 | pd127 |
| Tg(lepb up 7kb:EGFP) line 6 | pd128 |
| Tg(LENP2:EGFP) line 6 | pd129 |
| Tg(LENP2:EGFP) line 7 | pd130 |
| Tg(LENP2:EGFP) line 8 | pd131 |
| Tg(LEN(1-850)P2:EGFP) line 2 | pd132 |
| Tg(LEN(1-850)P2:EGFP) line C | pd133 |
| Tg(LEN(450-1000)P2:EGFP) line 2 | pd134 |
| Tg(LEN(450-1000)P2:EGFP) line A | pd135 |
| Tg(LEN(830-1350)P2:EGFP) line B | pd136 |
| Tg(LEN(830-1350)P2:EGFP) line E | pd137 |
| Tg(LEN(830-1350)P2:EGFP) line K | pd138 |
| Tg(LEN(450-850)P2:EGFP) line 11 | pd139 |
| Tg(LEN(450-850)P2:EGFP) line 12 | pd140 |
| Tg(LEN(450-660)P2:EGFP) line 1 | pd141 |
| Tg(LEN(450-660)P2:EGFP) line 3 | pd142 |
| Tg(LEN(570-720)P2:EGFP) line 1 | pd143 |
| Tg(LEN(660-850)P2:EGFP) line 2 | pd144 |
| Tg(LEN(660-850)P2:EGFP) line 5 | pd145 |
| Tg(LEN(660-850)P2:EGFP) line 6 | pd146 |
| Tg(LEN(1000-1350)P2:EGFP) line 3 | pd147 |
| Tg(LEN(1000-1350)P2:EGFP) line 7 | pd148 |
| Tg(LEN(1000-1350)P2:EGFP) line 14 | pd149 |
| Tg(LENcmlc2:EGFP) line 2 | pd150 |
| Tg(LENcmlc2:EGFP) line 12 | pd151 |
| Tg(LEN(1-850)cmlc2:EGFP) line 2 | pd152 |
| Tg(LEN(1-850)cmlc2:EGFP) line 3 | pd153 |
| Tg(LEN(450-1000)cmlc2:EGFP) line 6 | pd154 |
| Tg(LEN(830-1350)cmlc2:EGFP) line 4 | pd155 |
| Tg(LEN(450-850)cmlc2:EGFP) line 1 | pd156 |
| Tg(LEN(450-850)cmlc2:EGFP) line 2 | pd157 |
| Tg(LEN(450-850)cmlc2:EGFP) line 7 | pd158 |
| Tg(LEN(450-660)cmlc2:EGFP) line 1 | pd159 |
| Tg(LEN(570-720)cmlc2:EGFP) line 1 | pd160 |
| Tg(LEN(570-720)cmlc2:EGFP) line 12 | pd161 |
| Tg(LEN(570-720)cmlc2:EGFP) line 14 | pd162 |
| Tg(LEN(660-850)cmlc2:EGFP) line 2 | pd163 |
| Tg(LEN(1000-1350)cmlc2:EGFP) line 2 | pd164 |
| Tg(LEN(1000-1350)cmlc2:EGFP) line 5 | pd165 |
| Tg(LEN(1000-1350)cmlc2:EGFP) line 6 | pd166 |
| Tg(LENα-cry:EGFP) line 1 | pd167 |
| Tg(LENα-cry:EGFP) line 5 | pd168 |
| Tg(LEN(450-1000) α-cry:EGFP) line 1 | pd169 |
| Tg(LEN(450-1000) α-cry:EGFP) line 2-1 | pd170 |
| Tg(LEN(450-1000) α-cry:EGFP) line 2-3 | pd171 |
| Tg(LEN(450-1000) α-cry:EGFP) line 2-11 | pd172 |
| Tg(LEN(830-1350) α-cry:EGFP) line 6 | pd173 |
| Tg(LEN(830-1350) α-cry:EGFP) line 13 | pd174 |
| Tg(LENP2:fgf20a; efα:nls-mCherry) line 1 | pd175 |
| Tg(P2:fgf20a; ef1α:nls-mCherry) line 2 | pd176 |
| Tg(P2:fgf20a; ef1α:nls-mCherry) line 6 | pd177 |
| Tg(LENP2:dnfgfr 1-EGFP; ef1α:nls-mCherry) line 9 | pd178 |
| Tg(P2:dnfgfr 1-EGFP; ef1α:nls-mCherry) line 3 | pd179 |
| Tg(P2:dnfgfr 1-EGFP; ef1α:nls-mCherry) line 22 | pd180 |
| Tg(LENP2:nrg1; efα:nls-mCherry) line 10 | pd181 |
| Tg(P2:nrg1; ef1α:nls-mCherry) line 11 | pd182 |
| Tg(LEN(450-850-AP-1mut):P2:EGFP) line 3 | pd183 |
| Tg(LEN(450-850-AP-1mut):P2:EGFP) line 12 | pd184 |

To test additional TREES, we subcloned putative enhancer regions of i111a, plek, vcana, and cd248b upstream of 800 bp of lepb upstream sequence (P0.8). To define TREE activity, these constructs were injected into one-cell-stage wild-type embryos, Fin folds were amputated at 4 dpf, and eGFP fluorescence near the amputation plane was examined at 5 dpf (1 dpa).

TABLE 2
List of primers used in this study.

| Primer | Sequence |
|---|---|
| Lepb 5′ Homology arm Hind III-f | cgg aagctt tcagcaacttaagtgcattgat |
| lepb 5′ Homology arm BamHI-r | ccc ggatcc atttctgcaaaagaccaaatgaaa |
| lepb 3′ Homology arm NotI-f | c gcggccgc caacgatttaagcccatcat |
| lepb 3′ Homology arm KpnI-r | cc ggtacc actgcaaatcccatcaaaaa |
| lepb promoter EcoRI-r | ccc gaattc atttctgcaaaagaccaaatgaaa |
| lepb up 2 kb BamHI-f | ccc ggatcc tcttgaacaagtgactttcgttgca |
| lepb up 6 kb HindIII-f | cgg aagctt cttgtatgtttatacaagacacat |
| lepb enhancer (7 kb) HindIII-f | cgg aagctt actcgccaatttgatctgifict |
| lepb enhancer BamH-r | ggc ggatcc tggcatacacagcaaacatcatga |
| lepb enh 450-f HindIII | cgg aagctt agcagactttgaacccacaga |
| lepb enh 450-r BamHI | ccc ggatcc tctgtgggttcaaagtctgct |
| lepb enh 1000-f HindIII | cgg aagctt actgagaaggacaggaagct |
| lepb enh 1000-r BamHI | ccc ggatcc agcttcctgtccttctcagt |
| lepb enh 850-f HindIII | cgg aagctt aggaaattgctgcgtttccca |
| lepb enh 830-r BamHI | ccc ggatcc tgggaaacgcagcaatttcct |
| lepb enh 660-r BamHI | ccc ggatcc acgcgtttggtttttcataaaccac |
| lepb enh 660-f HindIII | cgg aagctt tcttatttttcagcattgtccttca |
| lepb enh 570-f HindIII | cgg aagctt aaatcacatcattccccgtctct |
| lepb enh 720-r BamHI | ccc ggatcc tgttttctacaccagaagagttca |
| cmlc2 ATG EcoRI-r | ccc gaattc ggagaagacattggaagagcct |
| cmlc2 1.6 kb BamHI-f | ggc ggatcc tgtaaatgagctctccaaatcagca |
| α-cry BamHI-f | ccc ggatcc ctta atatg gcaat aaacg atctt cagag a |
| α-cry EcoRI-r | ccc gaattc acagtacaaagatccccaaatgatgt |
| lepb RT-PCR-f | acaggatacgaatcattgctcga |
| lepb RT-PCR-r | tcagagaatgaatgtctcagcca |
| actb2 qPCR and RT-PCR-f | agagctacgagctgcctgac |
| actb2 qPCR and RT-PCR-r | taccgcaagattccataccc |
| lepb qPCR-f | gga tac gaa tca ttg ctc gaa |
| lepb qPCR-r | tct gga gac atc tgg aag tgc |
| nrg1 qPCR-f | cacaaatgagttcacatcacca |
| nrg1 qPCR-r | tctgctttgccattactcca |
| dnfgfr 1a ATG EcoRI-f | ggc gaattc atgataatgaagaccacgctgct |
| dnfgfr 1a 1.2 kb-r MfeI | ccc caattg agagctgtgcattttggccag |
| fgf20a ATG MfeI-f | ccc caattg atgggtgcagtcggcgagct |
| fgf20a stop XbaI-r | cc tctaga tcagctgtgacctagaacatcc |
| nrg1 ATG EcoRI-f | gcc gaattc atggctgaggtgaaagcagg |
| nrg1 stop XbaI-r | cc tctaga tcacacagctataggatcctg |
| ef1 α pro KpnI-f | cc ggtacc cagggggatcatctaatcaagc |
| ef1 α fragment PspomI-r | ctg gggccc gcagtgaaaaaaatgctttatttgtga |
| lepb ISH-f | agagattgatttcggccctgac |
| lepb ISH-r | cttgcatgtgccattgtgtttt |
| lepr 5′ Homology Arm SpeI-f | cc actagt caggccagtcttcagtcacg |
| lepr 5′ Homology Arm BamHI-r | ccc ggatcc cgt tgg att gat aca ttc act tcg a |
| lepr 3′ Homology Arm NotI-f | c gcggccgc tgtgctttgtaaggctgcac |
| lepr 3′ Homology Arm KpnI-r | cc ggtacc gtgctggtgcttcagaatga |
| AP-1 site mutant-f | aggaaagtttatgtgaatgcc tttc taa aaaaaa gttttcttca |
| AP-1 site mutant-r | actttaatgagatgaaattgaagaaaac tttttt tta gaaaggca |
| lepb up 800 bp BamHI-f | ggc ggatcc ttcgggcttgttgaaagggt |
| il11a-enh SpeI-f | cc actagt accatatcttgatacacgccaca |
| il11a-enh BamHI-r | ggc ggatcc tgacttcccaagccaaaacct |
| plek-enh HindIII-f | cgg aagctt tgggagcagaagtagaaacgt |
| plek-enh BamHI-r | ggc ggatcc acataccagtgaacacactcact |
| cd248b-enh HindIII-f | cgg aagctt tctcctcagttctccagatgt |
| cd248b-enh BamHI-r | ggc ggatcc acagcatgtttgggatgtggt |
| vcana-enh HindIII-f | cgg aagctt gctctgaggtgacagtgctt |
| vcana-enh BamHI-r | ggc ggatcc tctaggtacaaatggctctttga |

### Generation and Analysis of Transgenic Mice

Transgenic mice (CD-1 strain) were generated by oocyte microinjection as described previously.⁴⁴ LEN-hsp68::lacZ transgenic mice were generated by subcloning the zebrafish LEN enhancer sequence into the transgenic reporter plasmid hsp684acZ⁴⁵. Ctrl-hsp68::lacZ transgenic mice harbor a transgene, Prkaa2[mMEF2(1+2)]-hsp68-lacZ, which contains a modified version of a 931-bp enhancer sequence from the mouse Prkaa2 gene cloned into hsp68-lacZ (J. Hu and B. L. Black, unpublished observations). Partial apical resection injury in male and female neonatal mice at postnatal day 1 was performed similarly to previously described methods⁴⁶. Hearts and paws were collected at postnatal day 4. All experiments with mice complied with federal and institutional guidelines and were reviewed and approved by the UCSF IACUC.

### RNA Isolation and Quantitative PCR

RNA was isolated from dissected caudal tins and partially resected ventricles using Tri-Reagent (Sigma). cDNA was synthesized from 1 μg of total RNA using the Roche First Strand Synthesis Kit. Quantitative PCR was performed using the Roche LightCycler 480 and the Roche LightCycler 480 Probes Master. All samples were analyzed in biological triplicates and technical duplicates. Primer sequences are given in Table 2. Probe numbers for actb2, lepb, and nrg1 were 104, 156 and 76, respectively. lepb and nrg1 transcript levels were normalized to actb2 levels for all experiments.

### RNA Sequencing

Total RNA was prepared from two biological replicate pools of ablated Z-CAT ventricles and uninjured ventricles at 7 days post-ablation as per Gemberling et al.³¹, or regenerating and uninjured caudal fins. Generation of mRNA libraries and sequencing were performed at the Duke Genome Sequencing Shared Resource using an Illumina HiSeq2000. Sequences were aligned to the zebrafish genome (Zv9) using TopHat⁴⁷. Differentially regulated transcripts were identified using EdgeR and an FDR cut-off of O.1⁴⁸ Accession numbers for transcriptome datasets are GSE75894 and GSE76564.

### ChIP Sequencing

To identify candidate enhancer elements activated during heart regeneration, chromatin extracts were prepared from two biological replicate pools of 10 ablated Z-CAT ventricles and 10 uninjured ventricles. Chromatin was sonicated and immunoprecipitated with an antibody against H3K27ac (ActiveMotif) using the MAGnify ChIP system (Invitrogen). Sequencing libraries were prepared as per Bowman, et al.⁴⁹. Sequencing was performed using an Illumina HiSeq2000, and 10-25 million 50 bp single end reads were obtained for each library. Sequences were aligned to the zebrafish genome (Zv9) using Bowtie2⁵⁰. Differential peaks were identified using Model-based Analysis for ChiP-Seq (MACS)⁵¹.

### Histology and Imaging

In situ hybridization on cryosections of 4% paraformaldehyde-fixed fins was performed as described previously⁵². To generate digoxigenin-labeled probes for lepb and fgf20a, we generated a fragment of lepb cDNA and a full length of fgf20a cDNA by PCR using primer sequences described in Table 2. The nrg1 probe was prepared as described previously³¹. Immunohistochemistry was performed as described previously⁴⁰. Primary and secondary antibodies used in this study were: anti-Myosin heavy chain (mouse, F59, Developmental Studies Hybridoma Bank), anti-MEF2 (rabbit, sc-313, Santa Cruz Biotechnology), anti-PCNA (mouse, P8825, Sigma), anti-eGFP (rabbit, A11122, Life Technologies), anti-eGFP (chicken, GFP-1020. Ayes Labs), anti-Raldh2 (rabbit, Abmart), anti-Ds-Red (rabbit, 632496, Clontech), anti-p63 (mouse; 4A4, Santa Cruz Biotechnology), Alexa Fluor 488 (mouse and rabbit; Life Technologies), Alexa Fluor 594 (mouse and rabbit; Life Technologies). For EdU incorporation experiments, zebrafish were injected intraperitoneally with 10 mM EdU (A10055, sigma), and caudal fins were collected at 1 hour post-treatment. EdU staining was performed as previously described⁵³. The secondary antibody used for EdU staining was Alexa 488 azide (10-20 μM, Sigma). Whole-mount images were acquired using an M205FA stereofluorescence microscope (Leica) or Axio Zoom (Zeiss). Images of tissue sections (10 μm for hearts and 14 Am for fins) were acquired using an LSM 700 confocal microscope (Zeiss). X-gal staining to detect β-galactosidase activity and counterstaining with nuclear fast red were performed with murine tissue as described previously⁴⁴.

### Data Collection and Statistics

Clutchmates were randomized into different treatment groups for each experiment. No animal or sample was excluded from the analysis unless the animal died during the procedure. Sample sizes were chosen on the basis of previous publications and experiment types, and are indicated in each figure legend or methods. For expression patterns, at least five fish from each transgenic line were examined. At least 9 hearts of each group were pooled for RNA purification and subsequent RT-qPCR. Quantification of cardiomyocyte proliferation and calculation of statistical outcomes were assessed by a person blinded to the treatments. Sample sizes, statistical tests, and P values are indicated in the figures or the legends. One-way ANOVA tests were applied when normality and equal variance tests were passed. The Mann-Whitney rank sum test was applied in assays of cardiomyocyte proliferation.

### Example 1

### Modulation of Tissue Repair by Regeneration Enhancer Elements

How tissue regeneration programs are triggered by injury has received limited research attention. Here, we investigated the existence of enhancer regulatory elements that engage in regenerating tissue. Transcriptome analyses revealed that leptin b (lepb) is sharply induced in regenerating hearts and fins of zebrafish. Epigenetic profiling identified a short DNA sequence element upstream and distal to lepb that acquires open chromatin marks during regeneration and enables injury-dependent expression from minimal promoters. This element could activate expression in injured neonatal mouse tissues and was divisible into tissue-specific modules sufficient for expression in regenerating zebrafish fins or hearts. Simple enhancer-effector transgenes employing lepb-linked sequences upstream of pro- or anti-regenerative factors controlled the efficacy of regeneration in zebrafish. Our findings provide evidence for tissue regeneration enhancer elements (TREES) that trigger gene expression in injury sites and can be engineered to modulate the regenerative potential of vertebrate organs.

The capacity for complex tissue regeneration is unevenly distributed among vertebrate tissues and species. Salamanders and zebrafish possess remarkable potential to regenerate tissues like amputated appendages, resected heart muscle, and transected spinal cords^{1,2}. Investigations of gene expression arid function have generated molecular models for regeneration in multiple contexts, yet there is a deficiency in our understanding of the regulatory events that activate tissue regeneration programs¹⁻⁵.

Recent genome-wide chromatin analyses suggest that gene regulatory elements comprise a substantial portion of genomic sequence. Of these elements, distal-acting regulatory sequences, or enhancers, represent the most abundant class^{6,7}. Enhancers can direct expression of their target genes and have been predominantly examined as a means for stage- and tissue-specific regulation during embryonic development^{8,9}. Studies have also implicated enhancers in disease and as targets during evolution¹⁰⁻¹⁵. Because of such findings, it is possible there may also exist enhancer elements that engage in response to tissue damage to regulate genetic programs for regeneration. The identification of such elements could potentially inspire solutions for manipulating regenerative events.

### Example 2

### The Leptin Ortholog Leptin b is Induced in Regenerating Zebrafish Fin and Cardiac Tissues

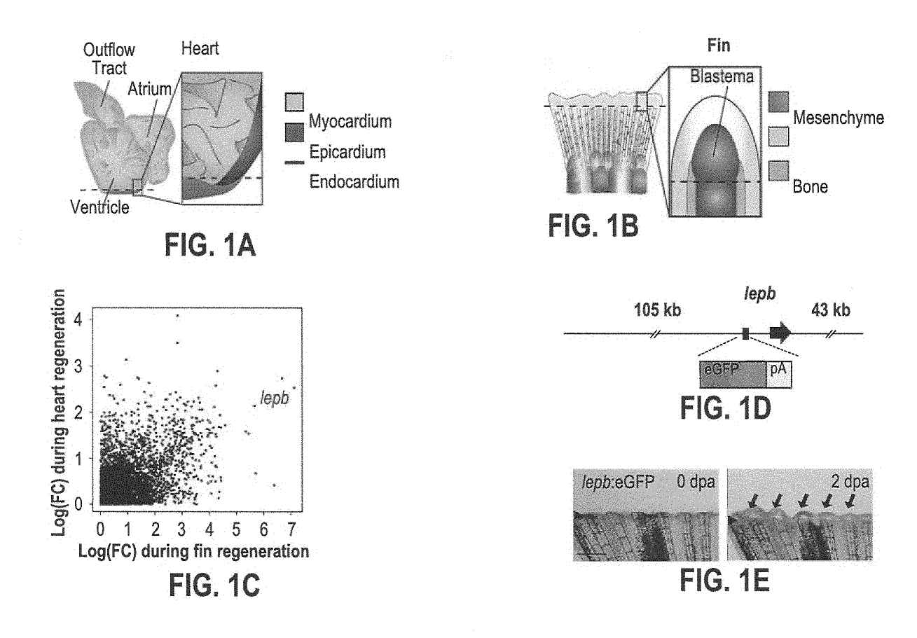

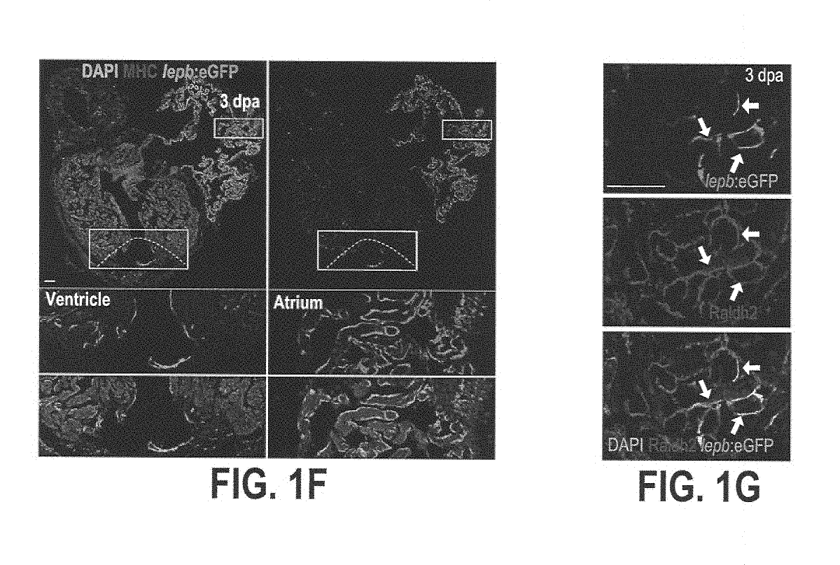

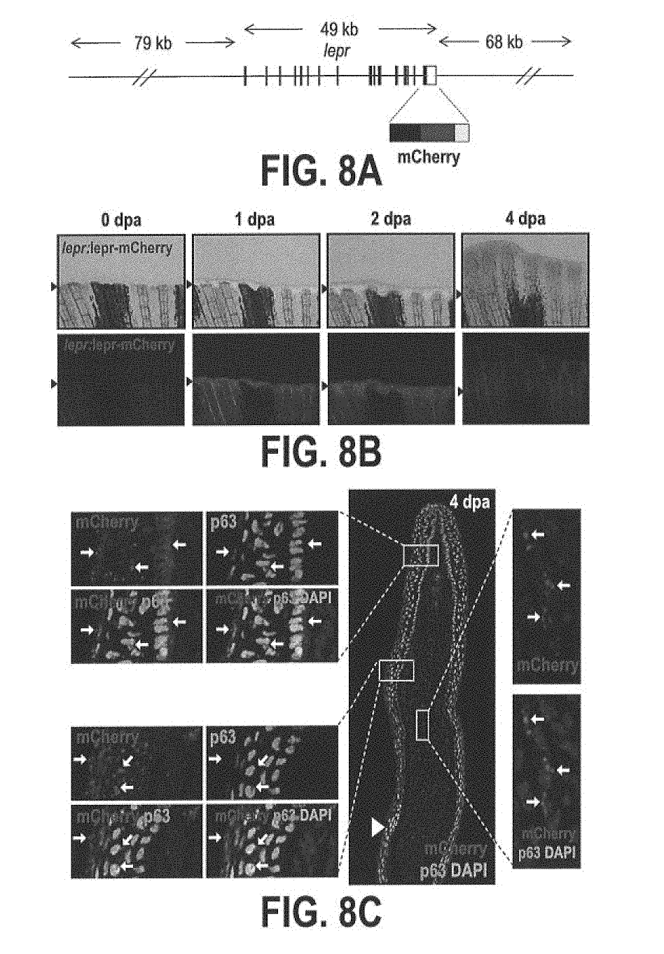

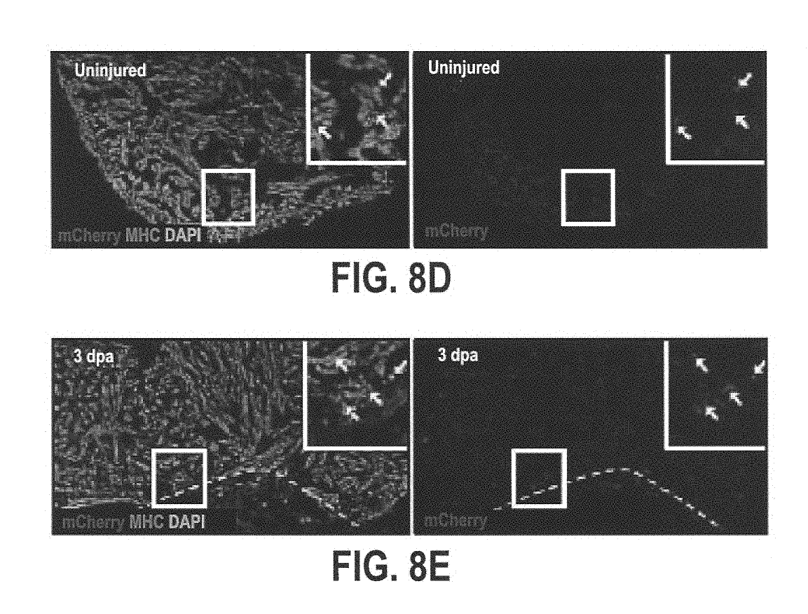

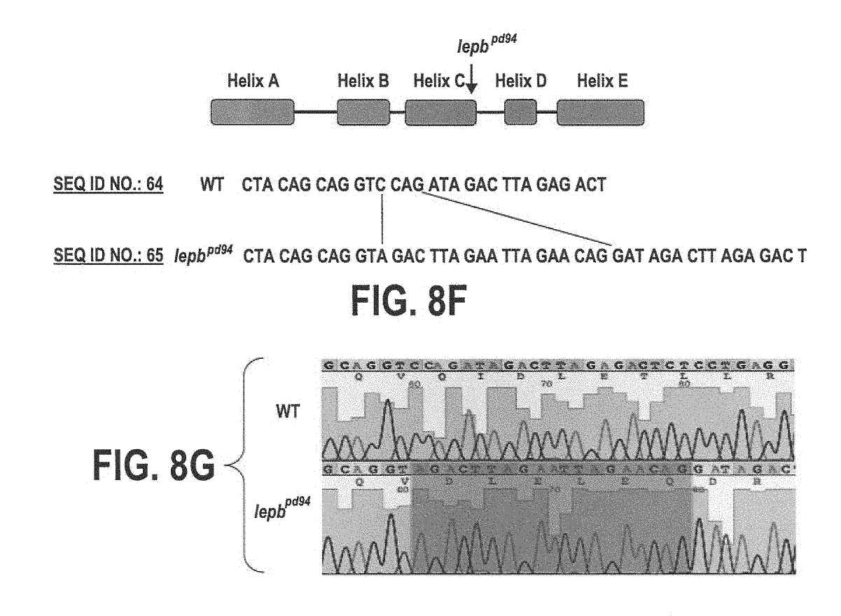

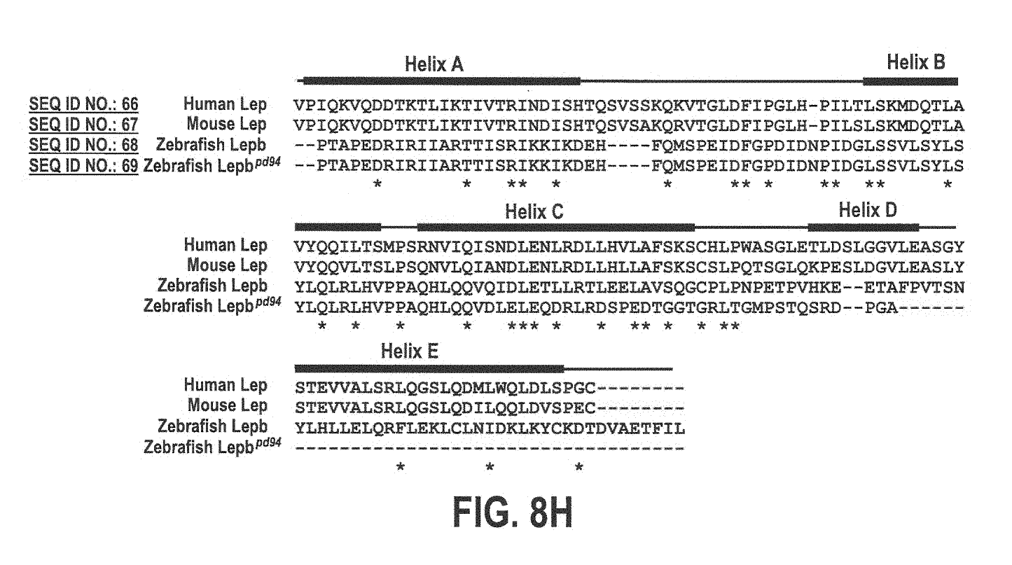

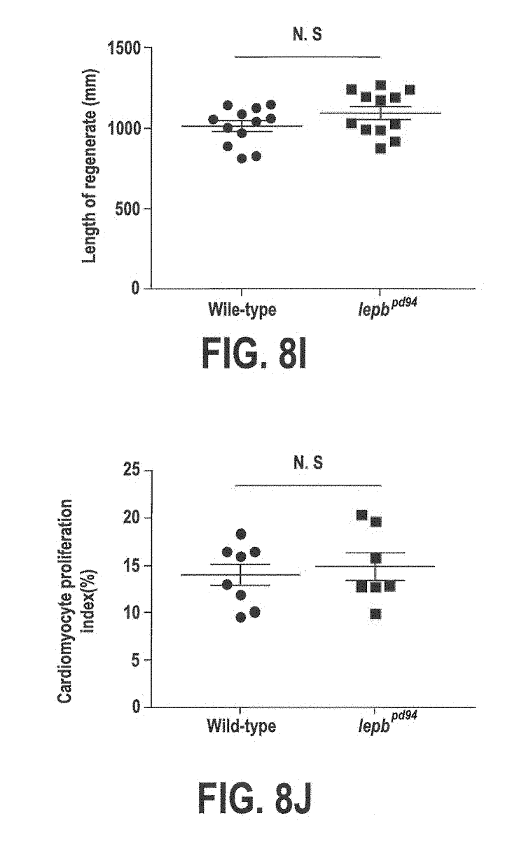

To identify genes that are induced during tissue regeneration, we collected RNA from uninjured and regenerating tissues of adult zebrafish and sequenced transcriptomes. Our analyses identified 2,408 genes with significantly higher expression in tail fins at 4 days post amputation (dpa), and 859 genes with significantly higher expression in cardiac ventricles 7 days after induced genetic ablation of half of all cardiomyocytes (FIG. 7A and Tables 4 and 5). In total, 360 genes were induced 2-fold or greater in both tissues compared to uninjured tissues (FIG. 7A). Among these genes, 69 were present at low levels in uninjured fins and induced sharply during regeneration (Table 3). leptin b (lepb), one of two zebrafish paralogs related to mammalian Leptin, a secreted regulator of energy homeostasis¹⁸, had the highest relative change during fin regeneration of genes in this group (130-fold; FIG. 1C, FIG. 8). lepb transcripts were rare or undetectable in uninjured fins by semi-quantitative or quantitative RT-PCR (qPCR) or in situ hybridization (ISH), but induced in the regeneration blastema by 1 dpa (FIGS. 7B to 7D). Upon local injury of the cardiac ventricle by partial resection, lepb expression was induced in the endocardium, the endothelial lining of inner myofibers that has been implicated in regenerative events (FIGS. 7B, 7C, and 7E)^{17,18}.

TABLE 3
List of 69 genes expressed at low levels in uninjured
fins and highly induced during regeneration.

| | Gene | | |
|---|---|---|---|
| ID | name | Fin fold change | Heart fold change |
| ENSDARG00000045548 | lepb | 139.5869041 | 5.783118267 |
| ENSDARG00000077938 | cd248b | 102.8405025 | 6.677217275 |
| ENSDARG00000078680 | vcana | 40.61925575 | 3.00391938 |
| ENSDARG00000078244 | si:ch211- | 30.81668987 | 3.45208716 |
| | 197l9.2 | | |
| ENSDARG00000037859 | il11a | 19.7766668 | 7.426410081 |
| ENSDARG00000055705 | f5 | 18.12716453 | 4.328098849 |
| ENSDARG00000069917 | ska3 | 15.45894566 | 2.78590812 |
| ENSDARG00000005993 | prc1b | 12.56607465 | 4.634054585 |
| ENSDARG00000011821 | plod2 | 12.53389624 | 2.643654683 |
| ENSDARG00000030759 | melk | 11.37272542 | 3.671438552 |
| ENSDARG00000043640 | cenpn | 11.18626299 | 2.875634469 |
| ENSDARG00000005098 | zgc:86764 | 10.74445202 | 2.751619994 |
| ENSDARG00000070239 | casc5 | 10.59047969 | 3.004360618 |
| ENSDARG00000055133 | cenpf | 10.34174394 | 3.248474193 |
| ENSDARG00000019128 | tpm4b | 10.30636139 | 3.147855145 |
| ENSDARG00000087198 | cthrc1a | 10.27259957 | 2.730617246 |
| ENSDARG00000045708 | adm2a | 10.18690641 | 4.371531065 |
| ENSDARG00000071658 | ywhag2 | 9.488818393 | 2.297710903 |
| ENSDARG00000052895 | htra3a | 8.939659332 | 4.456719971 |
| ENSDARG00000044541 | ppp1r14ba | 8.636167566 | 2.429573847 |
| ENSDARG00000060498 | tnfrsf9a | 8.420304443 | 2.214891479 |
| ENSDARG00000061928 | numa1 | 8.210282061 | 2.708742037 |
| ENSDARG00000056832 | exo1 | 8.099019702 | 2.891606096 |
| ENSDARG00000063285 | ube2t | 7.752886627 | 2.58817845 |
| ENSDARG00000058695 | ddr2b | 7.663768786 | 5.000777762 |
| ENSDARG00000070230 | aldh1l2 | 7.550329664 | 2.257319609 |
| ENSDARG00000035422 | cyr61l1 | 7.200408509 | 17.00903175 |
| ENSDARG00000075891 | sall1b | 7.139958125 | 4.178229406 |
| ENSDARG00000057323 | e2f8 | 6.748598744 | 2.503509739 |
| ENSDARG00000040178 | havcr1 | 6.707857872 | 2.908727734 |
| ENSDARG00000045219 | dkk1b | 6.512662603 | 2.9775671 |
| ENSDARG00000094634 | si:ch211- | 6.444918661 | 3.540647532 |
| | 120e1.7 | | |
| ENSDARG00000068457 | tnnt3b | 6.355595796 | 1.974852584 |
| ENSDARG00000003998 | phyhipla | 6.203215796 | 2.390450105 |
| ENSDARG00000021255 | arhgap22 | 6.188035541 | 3.528162449 |
| ENSDARG00000039062 | morn4 | 6.186386835 | 2.133581326 |
| ENSDARG00000079233 | e2f2 | 5.978313777 | 2.359654227 |
| ENSDARG00000013476 | arhgef39 | 5.901113746 | 3.073023944 |
| ENSDARG00000018623 | rad54l | 5.872877494 | 2.205835082 |
| ENSDARG00000075265 | plaua | 5.73231732 | 3.717899635 |
| ENSDARG00000009387 | robo4 | 5.45584801 | 2.071902332 |
| ENSDARG00000036505 | syt4 | 5.443425486 | 4.865203582 |
| ENSDARG00000002986 | gda | 5.256824084 | 4.838260109 |
| ENSDARG00000045802 | hapln3 | 4.972220662 | 2.043508849 |
| ENSDARG00000055226 | slc7a7 | 4.742748659 | 2.520351694 |
| ENSDARG00000025921 | runx1 | 4.125851137 | 2.941443776 |
| ENSDARG00000038541 | ccr12.3 | 3.72098279 | 3.801474567 |
| ENSDARG00000069912 | hmga2 | 3.535897936 | 2.953136892 |
| ENSDARG00000041381 | arntl2 | 3.462141193 | 6.220247187 |
| ENSDARG00000071491 | nrros | 3.457626556 | 2.036395481 |
| ENSDARG00000069017 | elnb | 3.445226442 | 2.673863059 |
| ENSDARG00000025855 | camk2n1a | 3.441189026 | 2.669102571 |
| ENSDARG00000075284 | kansl1l | 3.198552699 | 2.884446954 |
| ENSDARG00000038822 | mrc1b | 3.160791779 | 4.648139688 |
| ENSDARG00000009123 | sele | 3.125066749 | 2.918957853 |
| ENSDARG00000033662 | scd | 3.10073631 | 2.576725269 |
| ENSDARG00000077869 | dpy19l1l | 3.077532613 | 2.409904531 |
| ENSDARG00000062023 | fndc3bb | 2.757792103 | 2.038331473 |
| ENSDARG00000040027 | osbpl10 | 2.708239842 | 2.548537312 |
| ENSDARG00000061120 | slc43a2b | 2.687631028 | 2.885362714 |
| ENSDARG00000078125 | rusc1 | 2.525335688 | 2.409187806 |
| ENSDARG00000077054 | fam198b | 2.382947853 | 2.434447374 |
| ENSDARG00000017298 | plek | 2.364792092 | 3.959809914 |
| ENSDARG00000045544 | hgfa | 2.253544849 | 2.221888861 |
| ENSDARG00000013687 | cilp2 | 2.223615906 | 3.478056724 |
| ENSDARG00000076586 | csf2rb | 2.135476016 | 2.855436575 |
| ENSDARG00000043593 | rapgef1a | 2.124813912 | 2.11049704 |
| ENSDARG00000035907 | fam49a | 2.090088834 | 2.688615959 |
| ENSDARG00000057108 | serpine3 | 2.078496675 | 2.686004441 |

To capture the regulatory elements responsible for lepb induction, we replaced the first exon of lepb with an eGFP reporter transgene within a 150 kb BAC containing 105 kb of DNA sequence upstream of the start codon (FIG. 1D). Transgenic lepb_eGFP larvae had little or no detectable eGFP as viewed under a stereofluorescence microscope, and no fluorescence was detectable in fins or hearts throughout life (FIG. 1E and FIGS. 7I, 7L and 7M). Upon fin amputation, lepb:eGFP fluorescence was sharply induced in regenerating structures, where fluorescence localized to blastemal mesenchyme (FIG. 1E and FIGS. 7J and 7K). lepb:eGFP was also induced in wounds of resected ventricles, as well as in atrial tissue distant from the site of injury (FIG. 1G), a signature observed with other injury-induced markers^{17,18}. While sparse lepb:eGFP could be detected in epicardial tissue at 1 day post resection (dpa; data not shown), cardiac lepb:eGFP fluorescence was predominantly endocardial by 3 dpa (FIGS. 1G and 1H). Thus, sequences within a ˜150 kb genomic region surrounding lepb direct regeneration-dependent expression in fin and cardiac tissues.

### Example 3

### A Sequence Element Upstream of lepb is Sufficient for Regeneration-Activated Expression

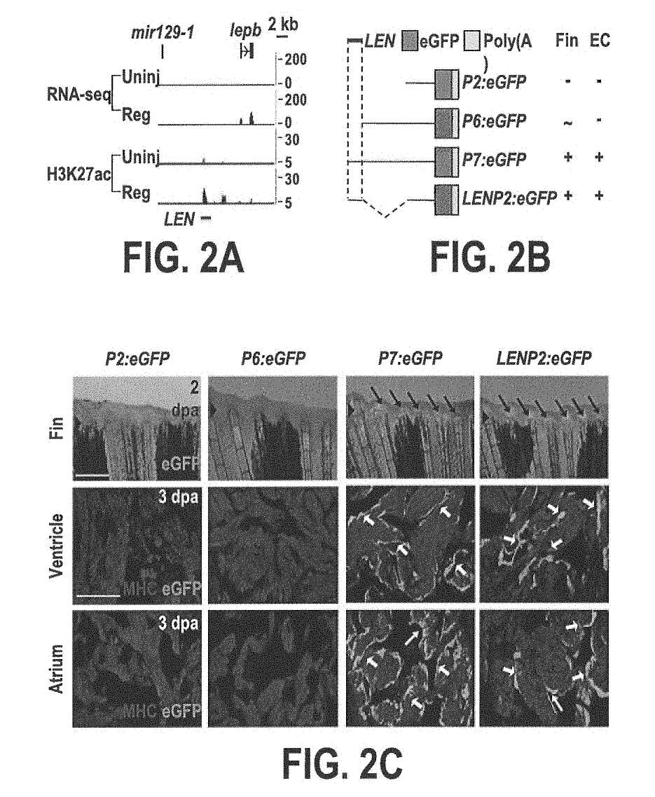

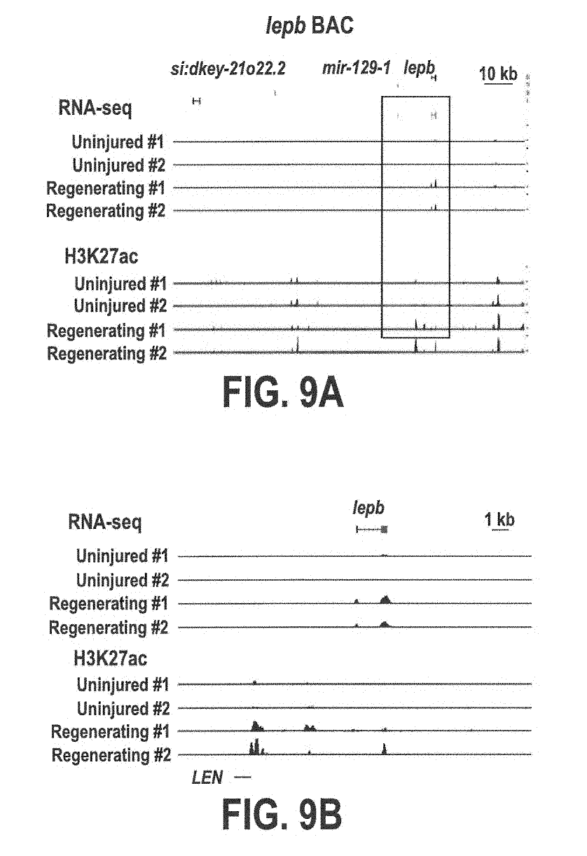

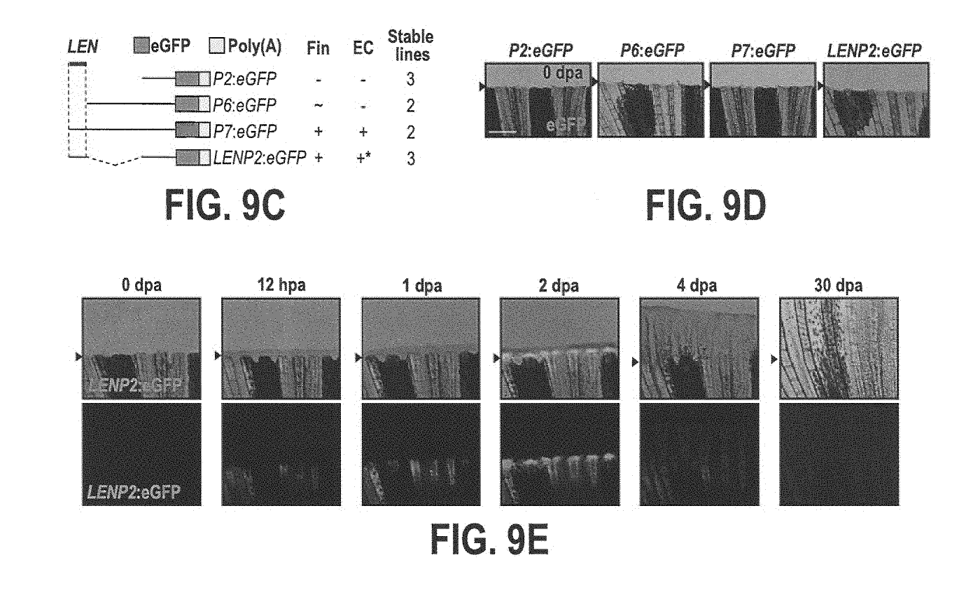

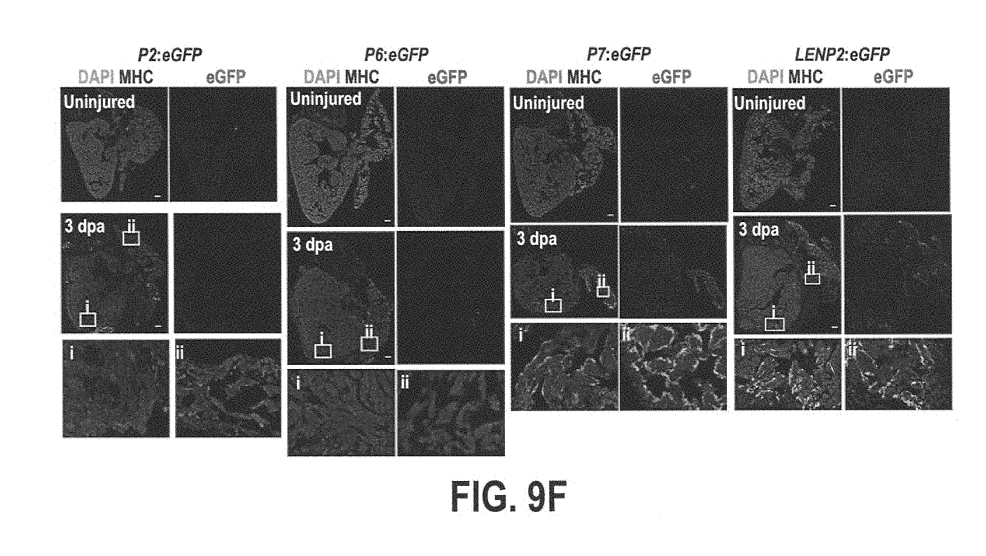

Enhancers are identifiable as areas of open chromatin, bound by transcription factors and occupied by histones possessing various modifications, such as acetylated lysine 27 of Histone H3 (H3K27ac)^{19,20}. To define areas of open chromatin, we assayed genomic regions surrounding lepb for H3K27ac marks by ChIP-Seq in samples of uninjured and regenerating hearts. Two regions within the lepb BAC, located 7 kb and 3 kb upstream of the lepb start codon, were enriched with H3K27ac marks in regenerating, but not uninjured, samples (FIG. 2A and FIGS. 9A and 9B). To examine if either of these distal regions exhibited enhancer activity, we established several transgenic lines containing 2 kb, 6 kb, and 7 kb upstream sequences of lepb fused to an eGFP reporter gene (referred to hereafter as P2:eGFP, P6:eGFP, and P7:eGFP) (FIG. 2B and FIG. 9C). Upon fin amputation, only P7:eGFP animals, with regulatory sequences encompassing the distal H3K27ac-rich area in the transgene, displayed strong blastemal expression that was comparable to lepb:eGFP BAC transgenic animals (FIG. 2C and FIG. 9D; P6:eGFP fins showed expression below the amputation site). Similarly, whereas P2:eGFP and P6:eGFP animals occasionally displayed induced fluorescence in myocardium and epicardium alter cardiac injury, only injured P7:eGFP hearts displayed strong endocardial fluorescence (FIG. 2C and FIG. 9F). Thus, a short DNA element located 7 kb upstream of the lepb coding sequence is important for directing gene expression in regenerating adult tissues.

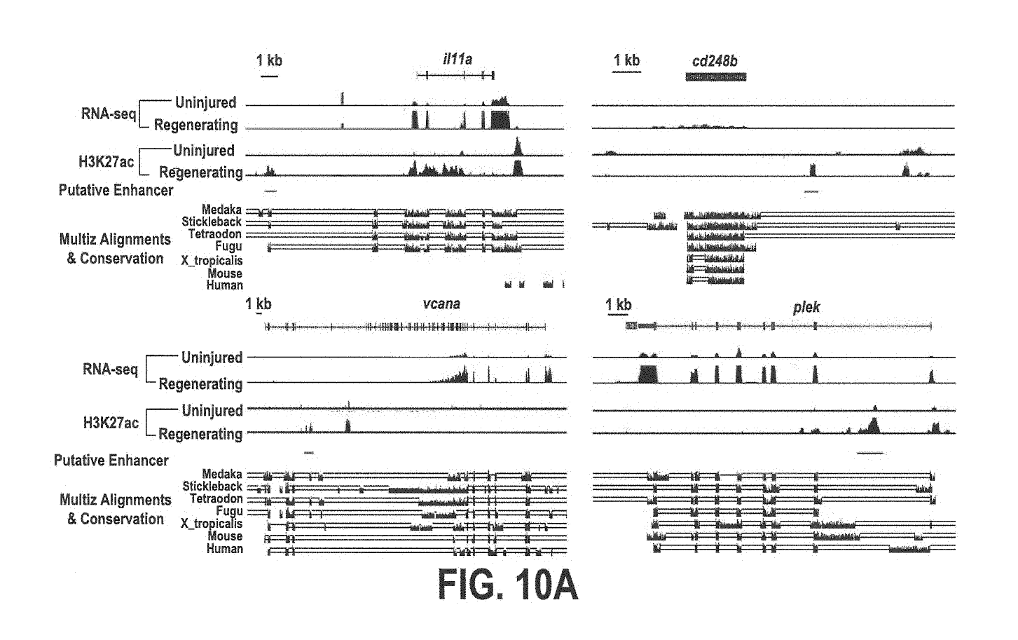

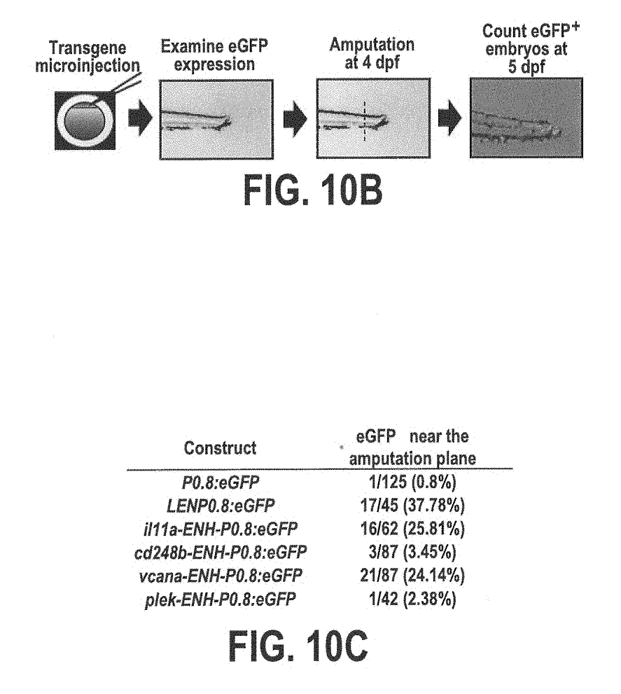

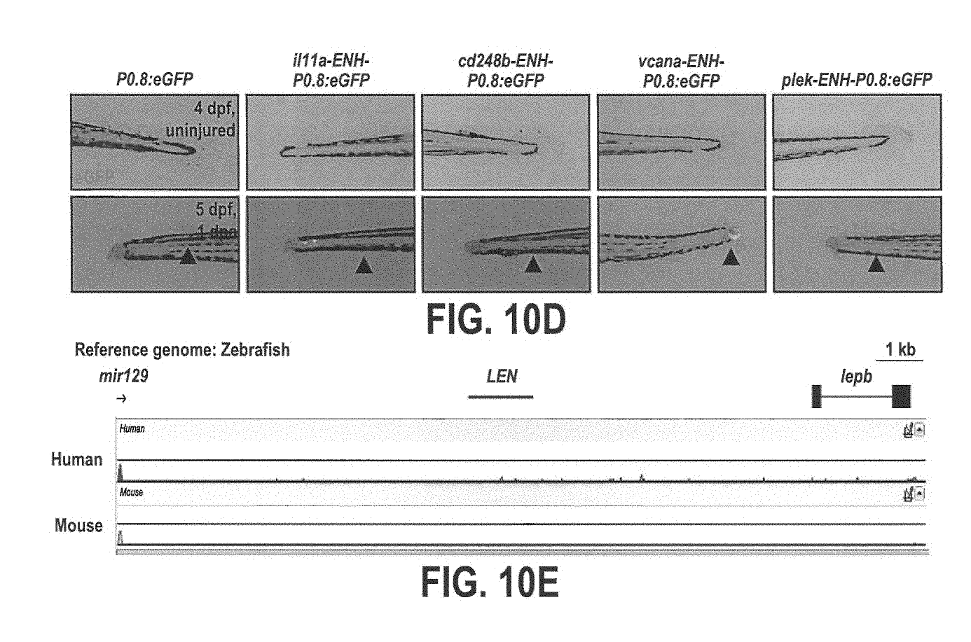

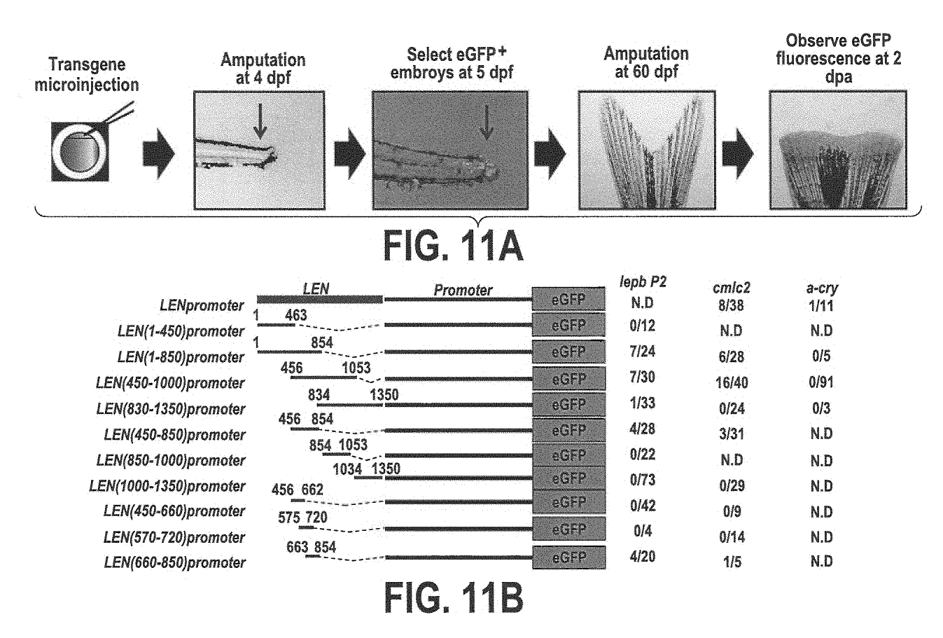

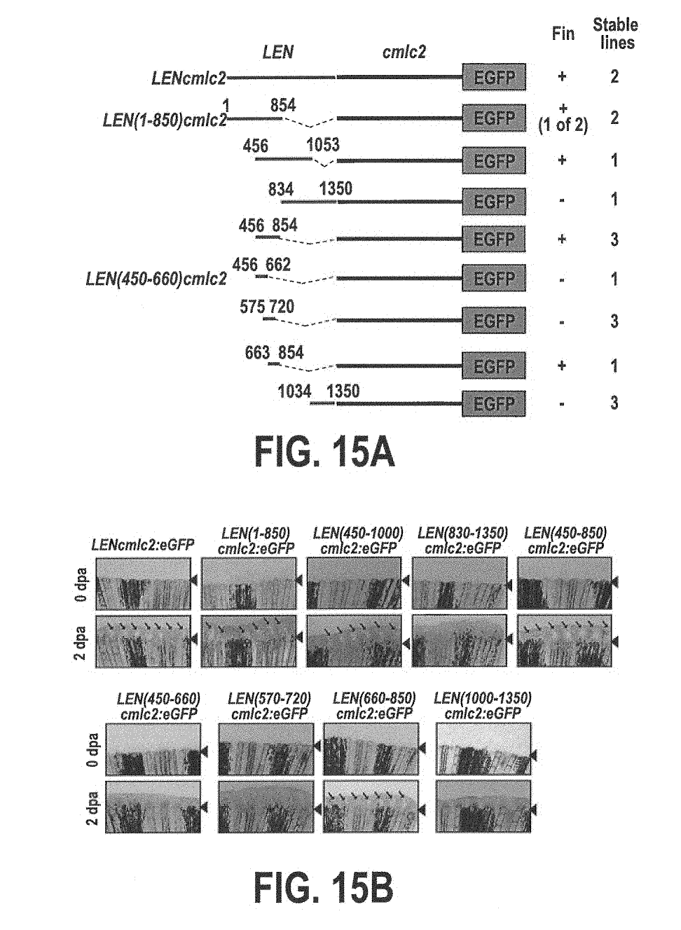

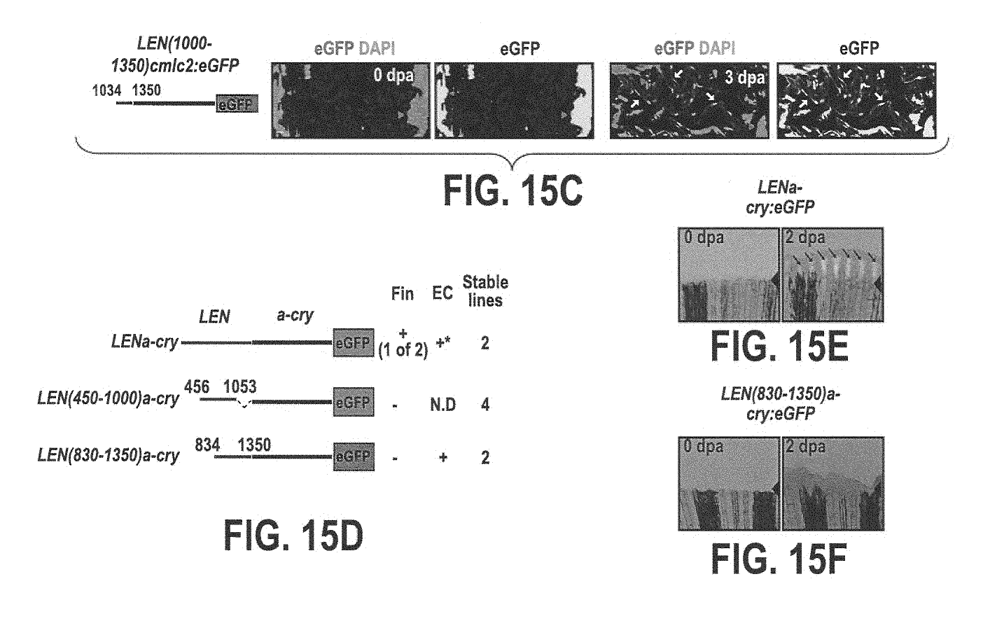

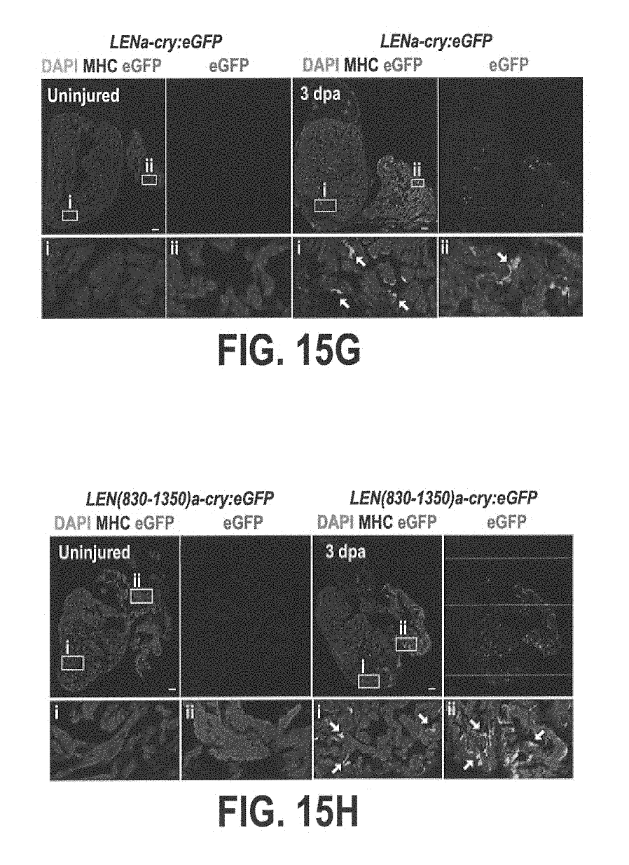

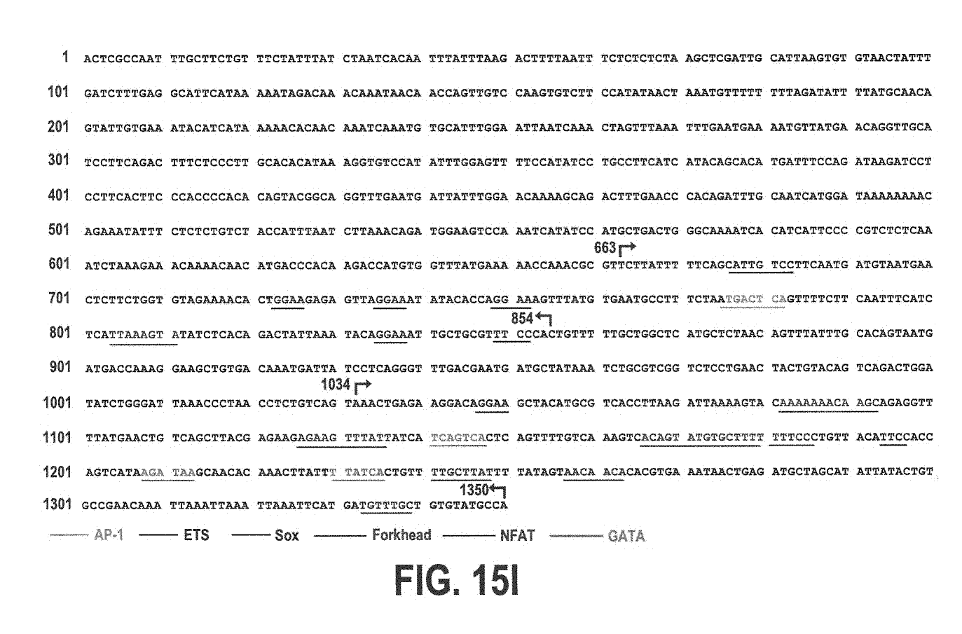

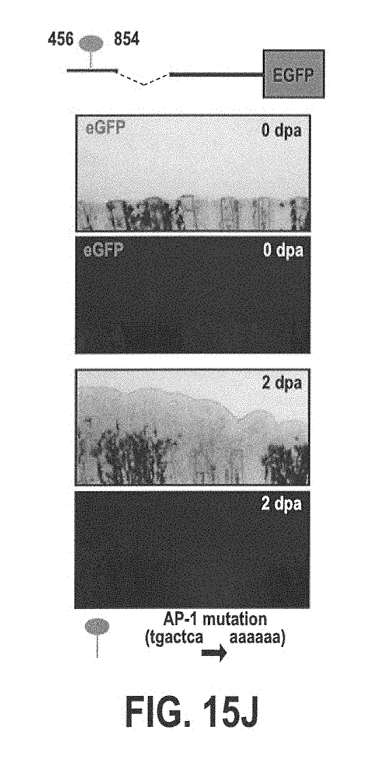

We next examined whether an isolated 1.3 Kb sequence that corresponded to the H3K27ac-rich region could activate gene expression when fused to P2, which ostensibly includes the lepb promoter (FIG. 2B and FIG. 9C). Although reporter eGFP fluorescence was not evident in uninjured adult fins or hearts of transgenic fish containing this lepb-linked distal element, fin amputation and ventricular resection activated eGFP fluorescence in blastemal and endocardial cells, respectively, in a similar manner to the lepb BAC sequences (FIG. 2C and FIGS. 9D to 9F). From a genome-wide H3K27ac survey, we also identified many 1-2 kb intergenic regions at other genomic loci that acquired H3K27ac marks during regeneration. We assessed sequence conservation and examined potential enhancer activity by transient transgenic reporter assays using several regions, some of which enabled expression from a minimal lepb promoter after injury (FIGS. 10A to 10D). To further validate the lepb -linked element, we examined its ability to influence the cell type-specific promoters cmic2 (cardiomyocytes) and α-cry (lens) in stable transgenic reporter lines. Robust, regeneration-dependent eGFP fluorescence was evident in fins and hearts of transgenic animals harboring either the cmic2 or α-cry promoters (FIG. 11 and FIGS. 15, 15B, 15D and 15G). Thus, a small intergenic element we now refer to as lepb-linked enhancer, or LEN, can direct regeneration-activated gene expression from multiple promoters.

### Example 4

### LEN-Associated Expression in Injured Neonatal Mouse Tissues

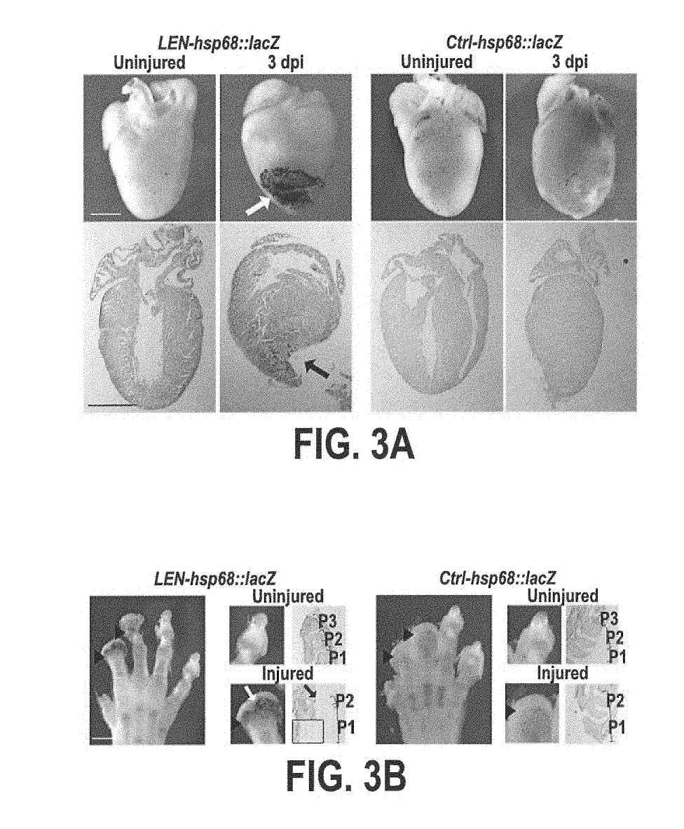

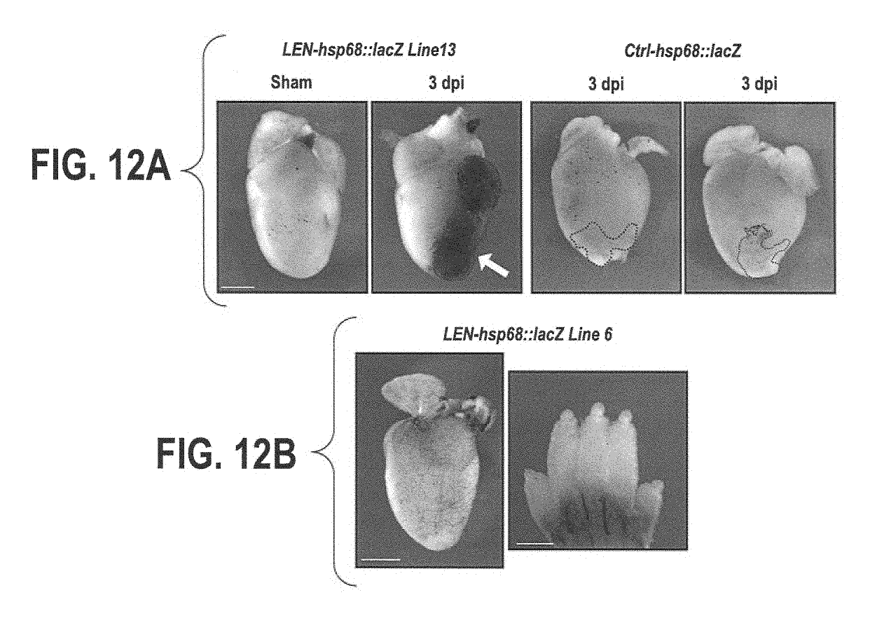

Analysis of regions upstream of Leptin genes in murine and human genomes revealed limited primary sequence conservation of LEN (FIG. 10E). This sequence divergence likely reflects rapid evolution of enhancers, reported in previous studies^{21,22}. To examine whether zebrafish LEN has activity in mammalian injury contexts, we fused it upstream of a construct containing a murine minimal hsp68 promoter and a lacZ reporter gene. We generated two stable lines, one of which displayed vascular endothelial X-gal staining in uninjured neonatal hearts and paws (FIG. 12B). A second line had a small number of X-gal-positive cells in uninjured neonatal tissues and was selected for injury studies (LEN-hsp68::lacZ) (FIGS. 3A and 3B). Neonatal digit tips amputated at P2 phalanges do not regenerate lost structures effectively²³, whereas injured neonatal ventricles display a regenerative response²⁴. Strikingly, amputated digit tips and damaged ventricles of all injured postnatal day 1 LEN-hsp68::lacZ neonates showed conspicuous X-gal staining in wounds 3 days following surgeries. A control transgenic line with an unrelated enhancer fragment also exhibited low basal expression in uninjured neonatal tissues, but unlike LEN-hsp68::lacZ animals, showed no detectable activation of the lacZ reporter upon injury to the digits or ventricle (FIGS. 3A and 3B and FIG. 12A). While tests of LEN activity using a panel of promoters and integration sites will be important, overall these results suggest that zebrafish LEN sequences can interact with mammalian transcriptional machinery to enable injury-induced expression in mice.

### Example 5

### The LEN Element is Separable into Tissue Specific Regulatory Modules

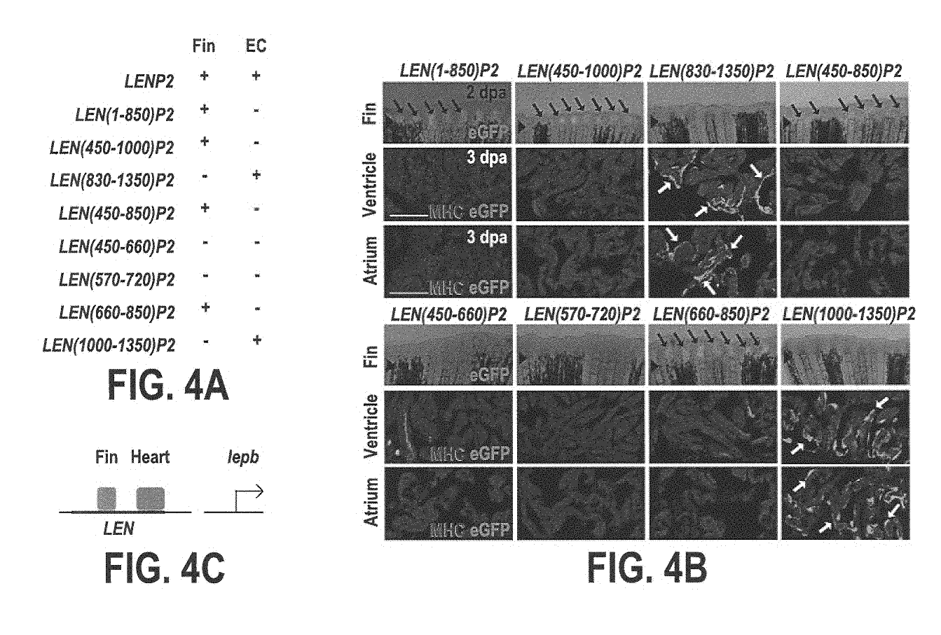

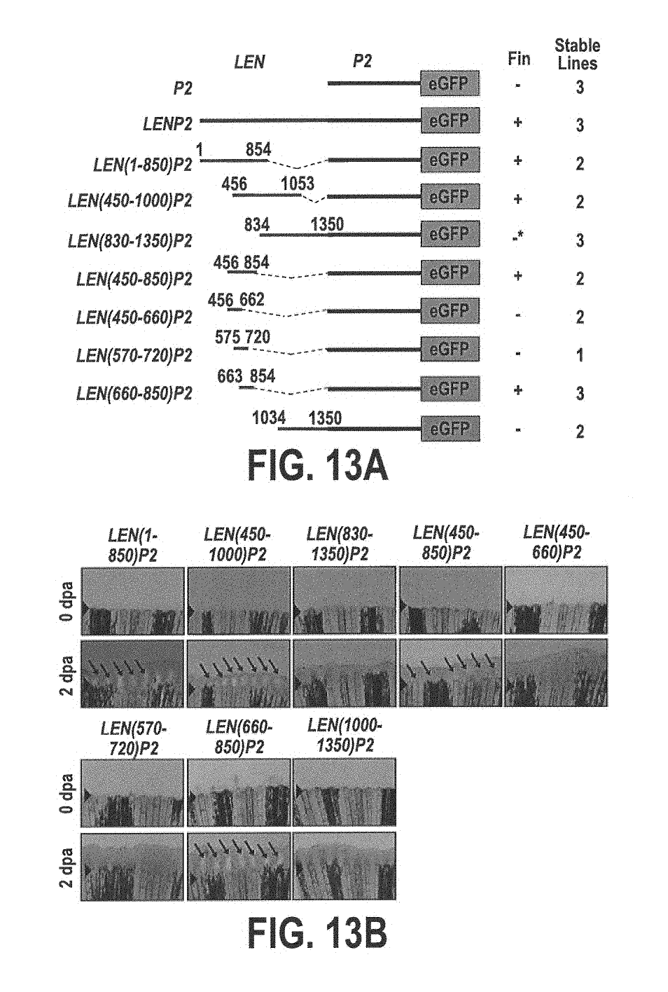

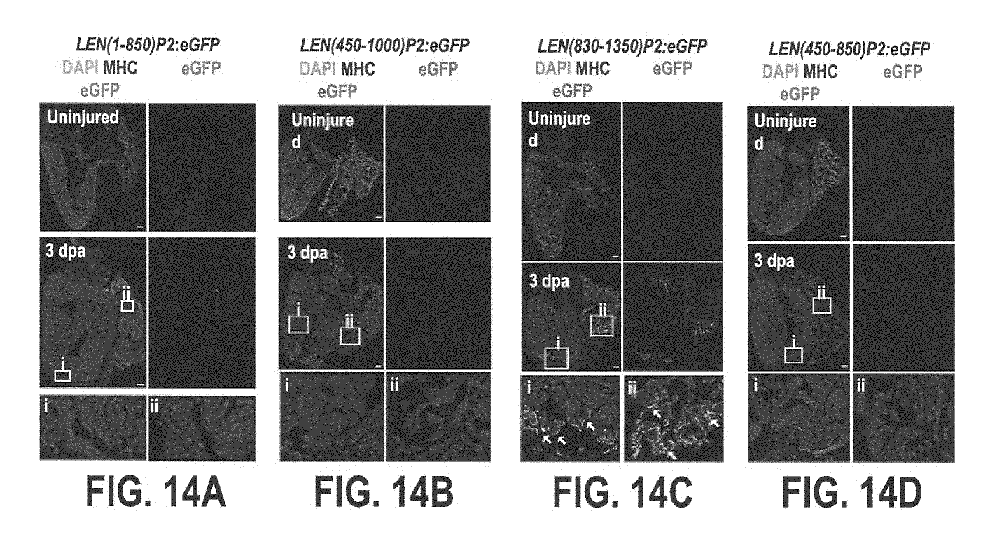

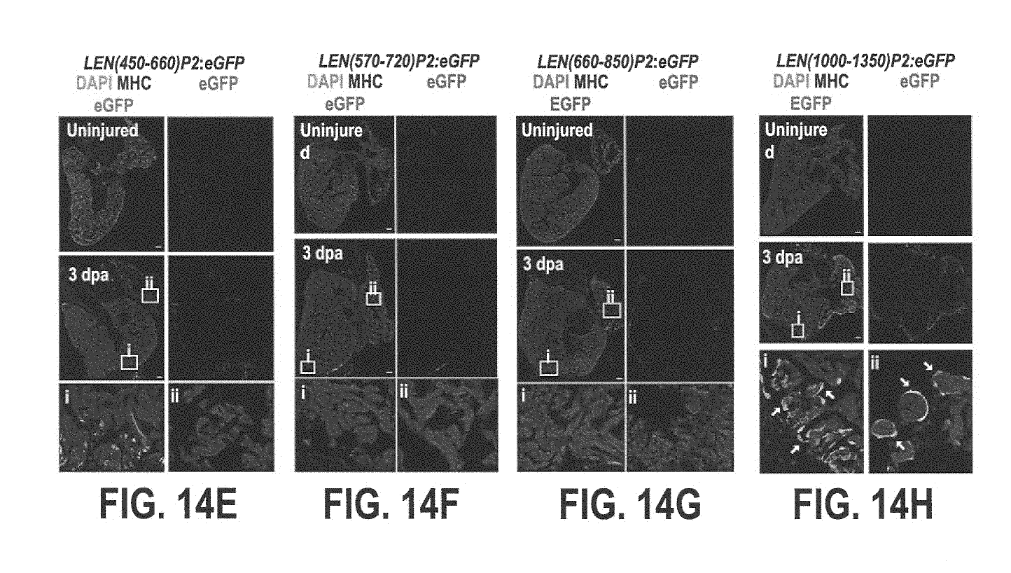

To identify minimal sequences responsible for the activity of LEN, we tested the ability of various fragments to direct regeneration-activated expression. We found that more distal LEN fragments composed of nucleotides 1-850, 450-1000, 450-850, or 660-850 could each drive eGFP expression from the lepb 2 kb promoter during fin regeneration (FIGS. 4A and 4B and FIG. 13). LEN fragments generated from the distal 1 kb portion also directed eGFP expression during fin regeneration when paired with the cmic2 promoter (FIG. 11 and FIGS. 15A and 15B). LEN fragments 1-850 and 450-1000 did not direct detectable eGFP expression during fin regeneration from the α-cry promoter in our experiments (FIG. 11 and FIGS. 15D to 15F), suggesting a repressive motif in α-cry upstream sequences. Intriguingly, none of these fragments directed endocardial expression alter cardiac injury, although eGFP fluorescence was occasionally observed sparsely in epicardial cells or cardiomyocytes (FIG. 14). Conversely, more proximal LEN fragments comprising nucleotides 830-1350 or 1000-1350 directed endocardial expression during heart regeneration, but did not activate eGFP fluorescence in regenerating fins (FIGS. 4A and 4B and FIGS. 13 and 14). These proximal LEN fragments also could direct regeneration-associated expression in endocardial cells from cmlc2 and α-cry promoters (FIGS. 15C and 15H). Thus, our analyses suggested the presence of two separate, tissue-specific enhancer modules (FIG. 4C).

We analyzed sequences of the minimal 190 nt (fin) and 316 nt (heart) elements, and identified distinct sets of predicted transcription factor binding motifs. LEN(663-854) contains predicted AP-1, Sox, Forkhead, and ETS binding sites, and we confirmed by transgenic reporter assays that a predicted AP-1 binding site at LEN(776-782) is necessary to direct expression in regenerating fins (FIGS. 15I and 15J). LEN(1034-1350) contains predicted NFAT, GATA, Forkhead, and ETS binding sites, motifs associated with expression in endothelial cells^{25,26} (FIG. 15I). In total, our findings indicate a composite arrangement of regulatory elements with distinct tissue preferences within the LEN regeneration enhancer.

### Example 6

### Engineered LEN Element Constructs can Control Regenerative Capacity

Recent studies have described new enhancer-target gene pairings caused by chromosomal rearrangements that underlie genetic diseases like cancer and neurological disorders^{10,12,15}. To examine a parallel idea for experimentally guiding tissue regeneration, we designed transgenic constructs positioning LEN and the minimal lepb promoter upstream of pro- or anti-regenerative factors. A possible outcome is that LEN would limit embryonic expression of potent developmental influences to permit maturation from the one-cell stage to adulthood, but also trigger and sustain expression of these influences upon tissue damage.

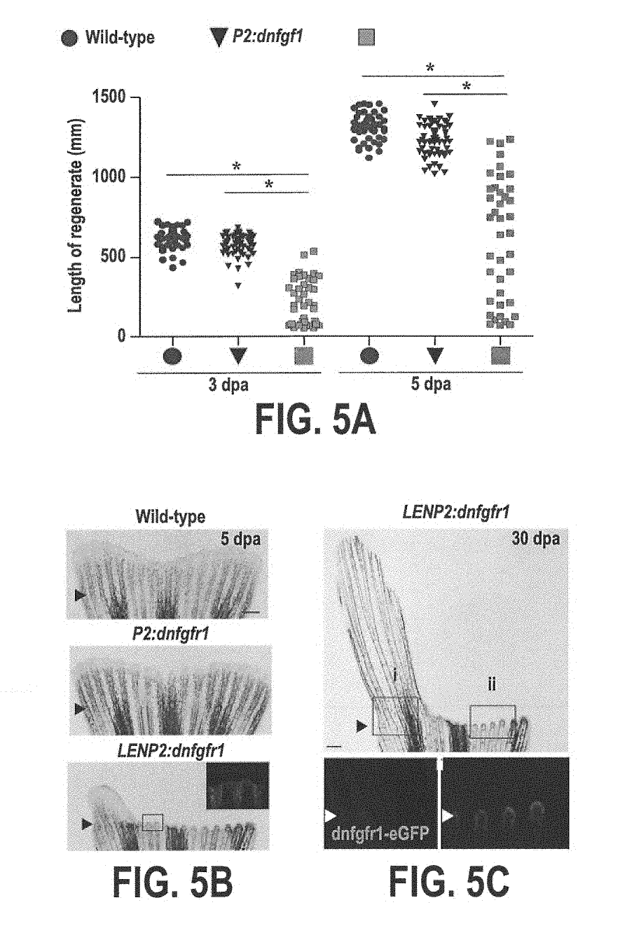

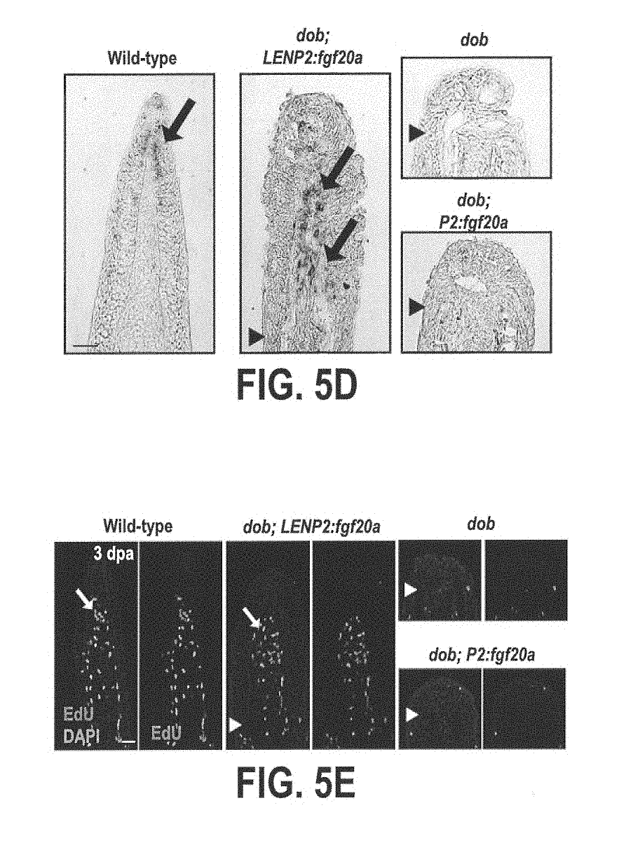

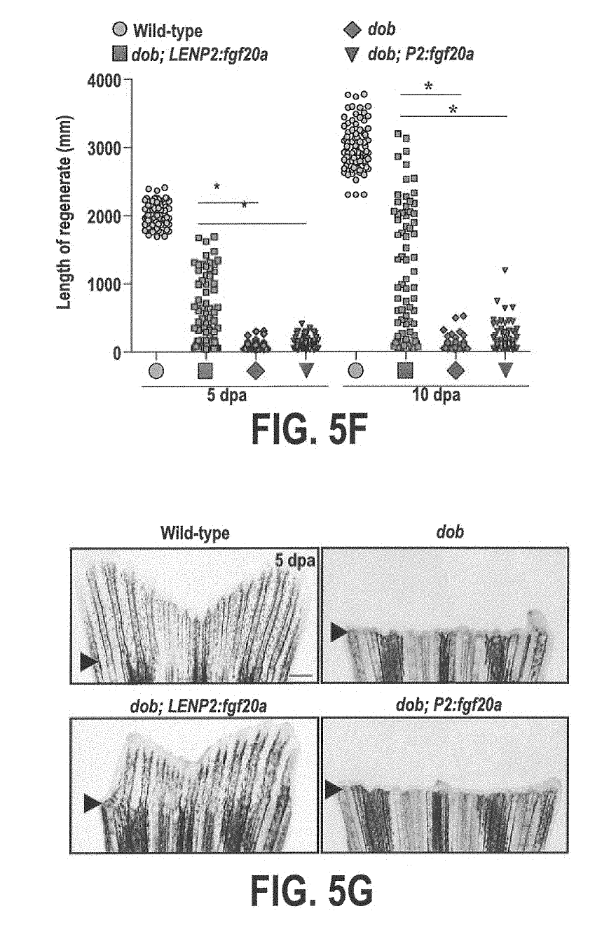

To create enhancer-effector transgenes, we took advantage of the dependency of fin regeneration on signaling by Fibroblast growth factors (Fgfs)^{4,27}. We first positioned LEN upstream of a cDNA encoding a dominant-negative form of fgfr1 (dnfgfr1)—a potent inhibitor of embryonic development^{27,28}—and injected this construct into wild-type embryos. We established stable lines of zebrafish harboring either P2:dnfgfr1 or LENP2:dnfgfr1, demonstrating that dnfgfr1 expression was limited to developmentally insignificant levels. Adult P2:dnfgfr1 fins displayed no detectable dnfgfr1 induction after amputation and regenerated normally. By contrast, injury to LENP2:dnfgfr1 animals induced strong expression of dnfgfr1 (detectable by dnfgfrl-eGFP fusion protein fluorescence) that was restricted to the amputation plane. Moreover, these animals displayed conspicuous defects or outright failures in fin regeneration (FIGS. 5A and B). In some cases, fin rays failed to regenerate even by 30 dpa and maintained dnfgfr1 expression in ray stumps, indicating persistent activation of LEN in the setting of regenerative failure (FIG. 5C and FIG. 16B).

We complemented these experiments with a gain-of-function approach, based on the discovery that mutations in the fgf20a ligand gene, devoid of blastema (dob), arrest fin regeneration⁴. We positioned LEN and the minimal lepb promoter upstream of a fgf20a cDNA and injected this construct into one-cell dob embryos. We generated stable lines of control dob; P2:fgf20a and dob; LENP2:fgf20a animals, indicating that these constructs restricted ectopic fgf20a expression during embryonic development. Upon amputation of adult tail fins, dob; P2:fgf20a animals induced no additional detectable fgf20a and displayed regenerative blocks comparable to dob animals (FIGS. 5D, 5F and 5G). By contrast, LENP2 sequences directed broad expression of fgf20a in mesenchymal cells upon fin amputation (FIGS. 5D, 5F and 5G). Remarkably, blastemal cell proliferation was stimulated in amputated dob; LENP2:fgf20a fins, and these animals regenerated patterned structures that were often of normal length (FIGS. 5E to 5G). In some cases, the lobed pattern of the tail fin was restored, and in no cases were there uncontrolled growth phenotypes (FIG. 5G).

### Example 7

### Targeted Stimulation of Cardiomyocyte Proliferation using the LEN Element

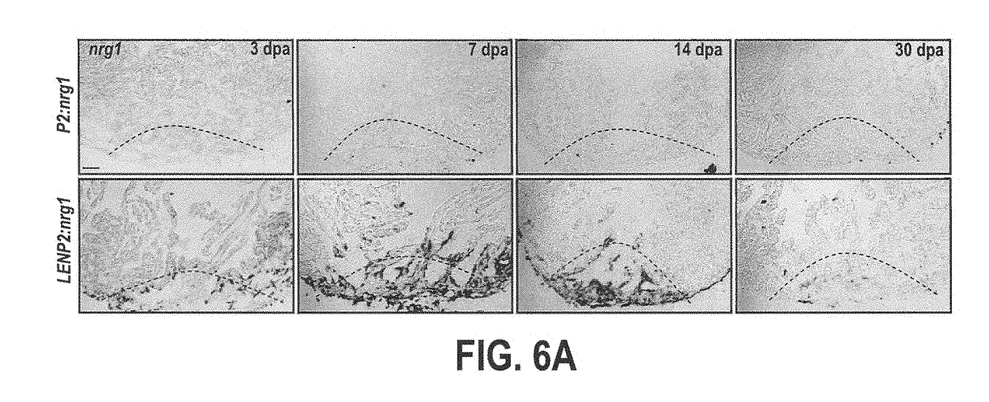

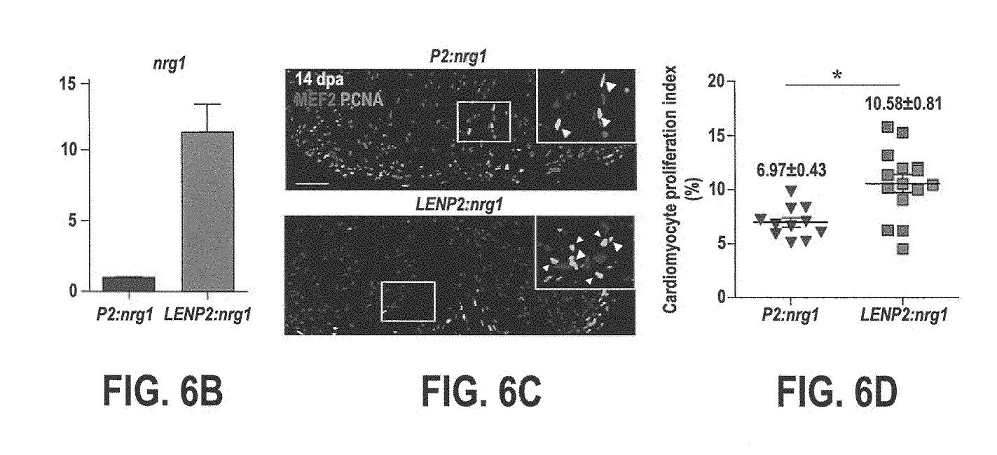

Heart regeneration occurs through injury-induced stimulation of proliferation by pre-existing cardiomyocytes²⁹. Recent evidence indicates that the secreted factor Neuregulin1 (Nrg1) is a cardiomyocyte mitogen during cardiac growth or repair in lower and higher vertebrates³⁰⁻³². In zebrafish, nrg1 is present at very low levels in the heart, and it is induced upon injury at levels that remain undetectable by standard ISH methodology³¹. Strong transgenic overexpression of nrg1 in adult zebrafish cardiomyocytes activates overt cardiomyocyte proliferation and enlarges the ventricular wall³¹. To test whether LEN can influence heart regeneration, we created stable transgenic zebrafish lines with P2:nrg1 or LENP2nrg1 constructs. Resection of the ventricular apex sharply increased nrg1 transcripts in injured portions of LENP2:nrg but not control P2:nrg1, ventricles (FIGS. 6A and 6B). LEN-induced nrg1 expression was strongest in 7 dpa injury sites, slightly less prominent at 14 dpa, and scarcely detectable by 30 dpa, typically when a contiguous muscle wall has regenerated (FIG. 6A). To examine effects of targeted nrg1 enhancement, we quantified cardiomyocyte proliferation indices in LENP2:nrg1 and P2:nrg1 ventricles at 14 dpa. LENP2:nrg1 injury sites had a 52% increase in cardiomyocyte proliferation compared to P2:nrg1 wounds, indicative of improved muscle regeneration (FIGS. 6C and 6D). By 30 dpa, when nrg1 levels approached baseline, regenerated ventricular walls appeared grossly normal (FIG. 6A). Thus, LEN can be designed to deliver mitogenic factors preferentially to areas of cardiac damage, boosting injury-induced cardiomyocyte proliferation.

Here, we used a profiling approach to identify small regulatory elements that direct gene expression in regenerating tissue, which we now refer to as Tissue Regeneration Enhancer Elements (TREES). Recently, a ˜18 Kb region of the murine Bmp5 locus was reported to activate expression from minimal promoters in injury contexts³³, suggesting it may harbor a TREE analogous to the LEN element we describe here. We suspect that diverse classes of TREES exist, including elements activated during development and re-activated by injury³⁴ or during regeneration, elements that activate expression preferentially during regeneration in multiple tissues, and regeneration-specific elements that are more tissue-restricted. The investigation of individual binding motifs within TREES should identify upstream transcriptional regulators of regeneration, whereas genomic TREE locations can pinpoint novel downstream target genes.

Current methodologies to interrogate regenerative biology often have experimental disadvantages like multiple transgenes, ubiquitous promoters, irreversible expression, and/or stressful stimuli like estrogen analogs, tetracycline analogs, or heat shock³⁵. By contrast, TREES are single-transgene systems that can naturally induce and maintain target genes upon injury, and then naturally temper expression as regeneration concludes. Whereas LEN elements induce expression in fin mesenchyme and/or endocardium, we expect that future investigations will uncover a panel of regeneration-responsive TREEs representing additional distinct tissues. Thus, when combined with effectors, recombinases, or genome-editing enzymes, TREEs should facilitate targeted genetic manipulations that have been elusive to this point.

Multiple features of TREEs are appealing with respect to the design of potential regenerative therapies. Previous studies have implicated the manipulation of enhancer activity as a means to treat human genetic disease^{12,36}. In this study, we report that pro-or anti-regenerative factors directed by TREEs are capable of blocking regenerative growth, promoting cell proliferation, or even rescuing genetic defects in regeneration. With a TREE-based system, factor delivery is spatiotemporally defined and could permit therapeutic cycles as injury recurs. Notably, although Nrg1 impacts heart regeneration, systemic neuregulin delivery has the potential for neurological or oncogenic effects^{37,38}. Thus, enhancer-based targeting of Nrg1 to injury sites, as we model here in zebrafish, may represent a more effective regenerative medicine platform. We suggest that TREES identified from natural regenerative contexts across vertebrate species can inform new strategies for precise factor delivery to injured human tissues.

### REFERENCES

1 Poss, K. D. Advances in understanding tissue regenerative capacity and mechanisms in animals. Nat. Rev. Gen. 11, 710-722, (2010).
2 Nacu, E & Tanaka, E. M. Limb regeneration: a new development? Ann. Rev. Cell Dev. Biol. 27, 409-440, (2011).
3 Kumar, A., Godwin, J. W., Gates, P. B., Garza-Garcia, A. A. & Brockes, J. P. Molecular basis for the nerve dependence of limb regeneration in an adult vertebrate. Science 318. 772-777, (2007).
4 Whitehead, G. G., Makino, S. Lien, C. L. & Keating, M. T. fgf20 is essential for initiating zebrafish fin regeneration. Science 310, 1957-1960, (2005).
5 Wehner, D. & Weidinger, G. Signaling networks organizing regenerative growth of the zebrafish fin. Trends Genet 31, 336-343, (2015).
6 Consortium, E P. An integrated encyclopedia of DNA elements in the human genome. Nature 489, 57-74, (2012).
7 Roadmap Epigenomics, C. et al, Integrative analysis of 111 reference human epigenomes. Nature 518, 317-330, (2015).
8 Lagha, M., Bothma, J. P. & Levine, M. Mechanisms of transcriptional precision in animal development Trends Genet 28, 409-416, (2012).
9 Visel, A., Rubin, E. M. & Pennacchio, L A. Genomic views of distant-acting enhancers. Nature 461, 199-205, (2009).
10 Giorgio, E. et al. A large genomic deletion leads to enhancer adoption by the lamin B1 gene: a second path to autosomal dominant adult-onset demyelinating leukodystrophy (ADLD). Hum. Mol. Genet. 24, 3143-3154, (2015).
11 Rebeiz, M., Pool, J. E., Kassner, V. A., Aquadro, C. F. & Carroll, S. B. Stepwise modification of a modular enhancer underlies adaptation in a Drosophila population. Science 326, 1663-1667, (2009).
12 van den Heuvel, A., Stadhouders, R., Andrieu-Soler, C., Grosveld, F. & Soler, E. Long-range gene regulation and novel therapeutic applications. Blood 125, 1521-1525, (2015).
13 Indjeian, V. B. et al. Evolving New Skeletal Traits by cis-Regulatory Changes in Bone Morphogenetic Proteins. Cell 164, 45-56, (2016).
14 Lonfat, N., Montavon, T., Darbellay, F., Gitto, S. & Duboule, D. Convergent evolution of complex regulatory landscapes and pleiotropy at Hox loci. Science 346, 1004-1006, (2014).
15 Herranz, D. et al. A NOTCH1-driven MYC enhancer promotes T cell development, transformation and acute lymphoblastic leukemia. Nat Med. 20, 1130-1137, (2014).
16 Zhang, Y. et al. Positional cloning of the mouse obese gene and its human homologue. Nature 372, 425-432. (1994).
17 Fang. Y. et al. Translational profiling of cardiomyocytes identifies an early Jakl/Stat3 injury response required for zebrafish heart regeneration. Proc. Natl Acad. Sci. USA, 110, 13416-13421, (2013).
18 Kikuchi, K. et al. Retinoic acid production by endocardium and epicardium is an injury response essential for zebrafish heart regeneration. Dev. Cell 20, 397-404, (2011).
19 Heintzman, N. D. et al. Histone modifications at human enhancers reflect global cell-type-specific gene expression. Nature 459. 108-112. (2009).
20 Creyghton, M. P. et al. Histone H3K27ac separates active from poised enhancers and predicts developmental state. Proc. Natl Acad. Sci. USA, 107, 21931-21936, (2010).
21 Villar, D. et al. Enhancer Evolution across 20 Mammalian Species. Cell 160, 554-566, (2015).
22 Blow, M. J. et al. ChIP-Seq identification of weakly conserved heart enhancers. Nat. Genet. 42, 806-810, (2010).
23 Simkin, J., Han, M., Yu, L., Yan, M. & Muneoka, K. The mouse digit tip: from wound healing to regeneration. Methods Mol. Biol. 1037, 419-435, (2013).
24 Porrello, E. R. et al. Transient regenerative potential of the neonatal mouse heart. Science 331, 1078-1080, (2011)
25 Park, C., Kim, T. M. & Malik, A. B. Transcriptional regulation of endothelial cell and vascular development. Circ. Res. 112, 1380-1400, (2013).
26 De Val, S. et al. Combinatorial regulation of endothelial gene expression by ets and forkhead transcription factors. Cell 135, 1053-1064, (2008).
27 Lee, Y., Grill, S., Sanchez, A., Murphy-Ryan, M. & Poss, K. D. Fgf signaling instructs position-dependent growth rate during zebrafish fin regeneration. Development 132, 5173-5183, (2005).
28 Amaya, E., Musci, T. J. & Kirschner, M. W. Expression of a dominant negative mutant of the FGF receptor disrupts mesoderm formation in Xenopus embryos. Cell 66, 257-270 (1991).
29 Kikuchi. K et al. Primary contribution to zebrafish heart regeneration by gata4(+) cardiomyocytes. Nature 464, 601-605, (2010).
30 Polizzotti, B. D. et al. Neuregulin stimulation of cardiomyocyte regeneration in mice and human myocardium reveals a therapeutic window. Sci. Translat Med. 7, 281ra245, (2015).
31 Gemberling, M., Karra, R., Dickson, A. L. & Poss, K. D. Nrg1 is an injury-induced cardiomyocyte mitogen for the endogenous heart regeneration program in zebrafish. eLife 4, (2015).
32 Bersell, K, Arab, S., Haring, B. & Kuhn, B. Neuregutin1/ErbB4 signaling induces cardiomyocyte proliferation and repair of heart injury. Cell 138, 257-270, (2009).
33 Guenther, C. A. et al. A distinct regulatory region of the Bmp5 locus activates gene expression following adult bone fracture or soft tissue injury. Bone 77, 31-41, (2015).
34 Huang, G. N. et al. C/EBP transcription factors mediate epicardial activation during heart development and injury. Science 338, 1599-1603, (2012).
35 Gemberting, M., Bailey, T. J., Hyde, D. R. & Poss, K. D. The zebrafish as a model for complex tissue regeneration. Trends Genet. 29, 611-620, (2013).
36 Deng, W. et al. Reactivation of developmentally silenced globin genes by forced chromatin looping. Cell 158, 849-860, (2014).
37 Nawa, H., Sotoyama, H., Iwakura, Y., Takei, N. & Namba, H. Neuropathologic implication of peripheral neuregulin-1 and EGF signals in dopaminergic dysfunction and behavioral deficits relevant to schizophrenia: their target cells and time window. Biomed Res. Int; 697935, (2014).
38 Montero, J. C. et al. Neuregulins and cancer. Clin. Cancer Res. 14, 3237-3241, (2008).
39 Poss, K. D., Wilson, L G. & Keating, M. T. Heart regeneration in zebrafish. Science 298, 2188-2190, (2002)
40 Wang, J. et al. The regenerative capacity of zebrafish reverses cardiac failure caused by genetic cardiomyocyte depletion. Development 138, 3421-3430, (2011).
41 Suster, M. L, Abe, G., Schouw, A. & Kawakami, K. Transposon-mediated BAC transgenesis in zebrafish. Nat Protocols 6, 1998-2021, (2011).
42 Burns, C. G. et al. High-throughput assay for small molecules that modulate zebrafish embryonic heart rate Nat Chem. Biol. 1, 263-264, (2005).
43 Kurita, R. et al. Suppression of lens growth by alphaA-crystallin promoter-driven expression of diphtheria toxin results in disruption of retinal cell organization in zebrafish. Dev. Biol. 255, 113-127 (2003).
44 Dodou, E, Xu, S. M. & Black, B. L. mef2c is activated directly by myogenic basic helix-loop-helix proteins during skeletal muscle development in vivo. Mech. Dev. 120, 1021-1032 (2003).
45 Kolhary, R. et al. Inducible expression of an hsp68-lacZ hybrid gene in transgenic mice. Development 105, 707-714 (1989).
46 Mahmoud, A. I., Porrello, E. R., Kimura, W., Olson, E. N. & Sadek, H. A. Surgical models for cardiac regeneration in neonatal mice. Nat Protocols 9, 305-311, (2014).
47 Trapnell, C., Pachter, L & Salzberg, S. L TopHat: discovering splice junctions with RNA-Seq. Bioinformatics 25, 1105-1111, (2009).
48 Robinson, M. D., McCarthy, D. J. & Smyth, G. K. edgeR: a Bioconductor package for differential expression analysis of digital gene expression data. Bioinformatics 26, 139-140, (2010).
49 Bowman, S. K et al. Multiplexed Illumina sequencing libraries from picogram quantities of DNA. BMC Genom. 14, 466, (2013).
50 Langmead, B. & Salzberg, S. L. Fast gapped-read alignment with Bowtie 2. Nat. Meth. 9, 357-359, (2012).
51 Zhang, Y., et al. Model-based analysis of ChIP-Seq (MACS). Gen. Biol. 9, R137, (2008).
52 Lee, V. et al. Maintenance of blastemal proliferation by functionally diverse epidermis in regenerating zebrafish fins. Dev. Biol. 331, 270-280, (2009).
53 Salic, A. & Mitchison, T. J. A chemical method for fast and sensitive detection of DNA synthesis in vivo. Proc Natl Acad. Sci. USA 105, 2415-2420, (2008).

### Example 8

### Transient Transgenic Analysis to Indicate Additional Regeneration Enhancers

To test whether peaks in our profiling datasets represented additional enhancer elements similar to LEN, we performed F₀ transgenic analysis. We used 800 nt of upstream sequence of lepb (P0.8) as a minimal promoter, as embryos injected with P0.8:EGFP only very rarely displayed EGFP fluorescence during fin fold regeneration (FIGS. 9B to 9D). We subcloned LEN and other enhancer candidate fragments upstream of P0.8:EGFP and injected these constructs (FIGS. 9A to 9D). Although F₀ mosaic transgenic embryos containing plek-ENH and cd248b-ENH fragments displayed no obvious regeneration-dependent enhancer activities, 37.78% (17/45), 25.81% (16/62), and 24.14% (21/87) of embryos injected with LENP0.8:EGFP, i111a-ENHP0.8:EGFP, and vcana-ENHP0.8:EGFP, respectively, displayed EGFP induction during fin fold regeneration (FIGS. 9C and 9D). These data suggest the existence of additional elements with similar properties of LEN.

### Example 9

### Transient Transgenic Analysis to Test Enhancer Activity in LEN Fragments During Fin Regeneration

To test enhancer activity during regeneration, we first analyzed EGFP transgene induction in F₀ mosaic transgenic animals. We fused LEN fragments to the lepb 2 kb upstream sequence (P2). After microinjection of LENP2 constructs, potential founders were selected at the larval (5 dpf) stage using an assay for induced EGFP fluorescence in response to fin fold amputation. At 60-90 dpf, caudal fins were amputated and EGFP fluorescence was examined (FIG. 10A). Although F₀ mosaic transgenic adult fish containing (850-1350), (1000-1350), (450-720), and (600-750) LEN fragments displayed no obvious enhancer activities, 29% (7/24), 23% (7/30), 14% (4/28), and 20% (4/20) of adults corresponding to LEN fragments (1-850), (450-1000), (450-850), and (660-850), respectively, displayed induced EGFP expression during fin regeneration. These data indicated that a fin regeneration enhancer element is located in LEN(663-854) (FIGS. 10B and 14I).

To examine whether LEN fragments are functional with different promoters, we tested enhancer activity with cmlc2 and α-cry promoters. A total of 21% (8/38), 21% (6/28), 40% (16/40), and 20% (1/5) of F₀ mosaic transgenic adult fish containing (1-1350), (1-850), (450-1000), and (660-850) LEN fragments coupled with the cmlc2 promoter, respectively, displayed enhancer activity during fin regeneration (FIG. 10B). By contrast, the α-cry promoter fused to (1-850) or (450-1000) LEN fragments displayed no activation of EGFP fluorescence during fin regeneration (FIG. 10B). These data suggest a possible repressive motif in the α-cry promoter that inhibits function of an enhancer during fin regeneration.

### Example 10

### Transient Transgenic Analysis to Test Modulation of Regenerative Capacity by Engineered LEN Constructs

To test whether the LEN element can be engineered to modulate tissue regeneration, we positioned LEN in constructs with a dominant-negative fgfr1, a potent inhibitor of fin regeneration. P2:dnfgfr1-EGFP and LENP2:dnfgfr1-EGFP constructs contained an ef1α-nls-mCherry mini-gene embedded in same vector, used as a screening marker. We injected these constructs into wild-type embryos, selected transgene-positive (red) embryos, and examined fin regeneration at 3 months of age. Whereas all 27 P2:dnfgfr1 F0 transgenic fish regenerated fins properly, 7 of 67 LENP2:dnfgfr1 F0 transgenic fish showed defective regeneration of some fin rays, corresponding to where LEN ectopically induced dnfgfr1-EGFP (FIG. 16A).

To examine whether a LEN element coupled with the Fgf ligand gene fgf20a could rescue the defective fin regeneration in dob mutants, we injected two constructs into dob embryos: 1) fgf20a downstream of the lepb 2 kb promoter, with ef1α-nls-mCherry as a screening marker (dob; P2:fgf20a); and 2) this same construct with a LEN sequence positioned upstream of P2 (dob; LENP2:fgf20a). We examined fin regeneration in transgene-positive (red) F₀ mosaic adult transgenic fish. Whereas 2 of 19 dob, and 2 of 40 dob; P2:fgf20a F₀ animals regenerated more than 500,000 μm² of tissue at 10 dpa, 11 of 44 dob; LENP2:fgf20a F₀ animals regenerated more than 500,000 μm² (FIGS. 16C and 16D). One obvious phenotype of dob is the absence of a clear blastema, generally distinguishable histologically and as a dense region of EdU labeling. Although the blastema is rarely detectable in sectioned samples of dob; P2:fgf20a F₀ fins, many dob; LENP2:fgf20a F₀ fin regenerates displayed recognizable blastema formation (FIG. 16E). Moreover, by in situ hybridization, we confirmed ectopic expression of fgf20a in mesenchymal cells of dob; LENP2:fgf20a F₀ fin regenerates, signals that were not detected in dob; P2:fgf20a F₀ fin tissues (FIG. 16F).

### Example 11

### BLAST Results with LEN Fragment

To determine whether enhancer elements related to LEN are found near genes that show significantly higher levels during regeneration than in uninjured tissues, we performed BLAST using fin and endocardial LEN elements. Blast analysis revealed genomic regions that show homology with LEN near some genes, listed in Tables 4 and 5 below.

TABLE 4
Blast analysis with 1034-1307 (Endocardial enhancer elements).

| | | | | | Fold | Fold |
|---|---|---|---|---|---|---|
| | Homologous | | | | change | change |
| Locus | sequence | Identities | Gene | Distance | in heart | in fin |
| 4: | TAAAAAAAAACAA | 23/25 | tfec | 238 kb | 2.23 | 1.5 |
| 6226627- | GCAGAGGTATTA | | | upstream | | |
| 6226651 | | | | | | |
| 3:57589360- | AAGATTACCAAAA | 41/53 | timp2b | intron | 4.14 | 5.34 |
| 57589412 | TAAACTTATTTT | | | | | |
| | AAAACTTTTTATTT | | | | | |
| | TTTATTTTATAATA | | | | | |
| 21:28381348- | AAGATTAAAACT- | 33/39 | rasgrp2 | intron | 2.68 | 2.82 |
| 28381312 | CAAAATAA-AAGC | | | | | |
| | CAAGGTTTTATGA | | | | | |

TABLE 5
Blast analysis with 697-854 (Fin enhancer elements)

| | | | | | Fold | Fold |
|---|---|---|---|---|---|---|
| | Homologous | | | | change | change |
| Locus | sequence | Identities | Gene | Distance | in heart | in fin |
| 5:44207133- | AGTTTTCCTT | 30/34 | ctsla | intron | 0.91 | 4.51 |
| 44207100 | CAACTTTTAT | | | | | |
| | TTCATTAAAG | | | | | |
| | TATA | | | | | |
| 24:25302951- | CAGGAAAGT | 23/25 | phex | 5.5 kb | 4.81 | 7.1 |
| 25302975 | TTATGTAAA | | | upstream | | |
| | TGACTTT | | | | | |
| 24:16377889- | TTTCACTTCT | 25/28 | sema5a | intron | 0.42 | 1.97 |
| 16377862 | CATTAAAGTA | | | | | |
| | TATCTGAC | | | | | |
| 8:7333308- | ATCTCATTAAA | 27/31 | ssuh2rs1 | 3 kb | 1.42 | 2.39 |
| 7333338 | GTATGATCTTA | | | downstream | | |
| | TAGACAATT | | | | | |

8. Analysis of lepr:lepr-mCherry Reporter and lepb Mutant Lines.

To visualize which cells might respond to Lepb during tissue regeneration, we created a lepr BAC reporter line. To generate lepr:lepr-mCherry BAC transgenic animals (formal name Tg(lepr:lepr-mCherry)^{pd95}), the iTol2 cassette was integrated into the BAC clone DKEY-1K24. Then, a mCherry cassette was integrated at the C-terminus of Lepr, which results in a C-terminal mCherry-fusion of Lepr. (FIG. 8A). Upon fin amputation, Lepr-mCherry fluorescence was induced in the fin regenerate at the distal, regenerating structures at 1 and 2 dpa and decreased at 4 dpa (FIG. 8B). Confocal section images of 4 dpa fin samples indicated that epithelial cells and putative vascular cells predominantly express Lepr-mCherry (FIG. 8C). Lepr-mCherry fluorescence was also detectable in cardiomyocytes in the uninjured heart (FIG. 8D), and its expression was retained in cardiomyocytes after ventricular resection (FIG. 8E). In mammals, Leptin signaling has been reported to promote wound healing after skin damage¹⁻³ and angiogenesis⁴, and has been proposed to play a cardioprotective role^{5,6}.

To examine requirements for lepb in zebrafish tissue regeneration, we used TALE Nucleases to generate mutations in the a-helix C of Lepb (FIG. 8F), and we established homozygous mutants for a frameshift, truncating mutation in lepb (pd94) (FIGS. 8F to 8H). lepb^{pd94} animals are viable, and we did not detect any apparent obese phenotype under standard husbandry conditions. To examine whether lepb^{pd94} causes defects in tissue regeneration, we measured lengths of regenerating fins 4 days after fin amputation, and quantified cardiomyocyte proliferation at 7 days after partial ventricular resection. lepb mutants regenerated grossly normally after fin amputation and showed normal injury-induced cardiomyocyte proliferation FIGS. 8I and 8J). This result is most likely explained by: 1) a lack of major involvement in regeneration by lepb; or 2) compensation by the lepa paralogue or other Jak/Stat ligands such as I111a that are induced during regeneration^{7,8}. A recent study has highlighted the capacity of zebrafish to compensate for genetic mutations (Rossi et al. 2015)⁷.

### SUPPLEMENTARY REFERENCES

1 Frank, S., Stallmeyer, B., Kampfer, H., Kolb, N. & Pfeilschifter, J. Leptin enhances wound re-epithelialization and constitutes a direct function of leptin in skin repair. J. Clin. Invest. 106, 501-509, (2000).
2 Tadokoro, S. et al. Leptin promotes wound healing in the skin. PLoS One 10, e0121242, (2015).
3 Umeki, H. et al. Leptin promotes wound healing in the oral mucosa. PLoS One 9, e101984, (2014).
4 Sierra-Honigmann, M. R. et al. Biological action of leptin as an angiogenic factor. Science 281, 1683-1686 (1998).
5 McGaffin, K. R. et al. Leptin signaling reduces the severity of cardiac dysfunction and remodeling after chronic ischaemic injury. Cardiovas. Res. 77, 54-63, (2008).
6 Matsui, H. et al. Ischemia/reperfusion in rat heart induces leptin and leptin receptor gene expression. Life Sci. 80, 672-680, (2007).
7 Rossi, A. et al. Genetic compensation induced by deleterious mutations but not gene knockdowns. Nature 524, 230-233, (2015).
8 Fang, Y. et al. Translational profiling of cardiomyocytes identifies an early Jak1/Stat3 injury response required for zebrafish heart regeneration. Proc. Natl Acad. Sci. USA 110, 13416-13421, (2013).

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein. All patents and publications cited herein are incorporated by reference herein for the specific teachings thereof as noted, unless other specific statements of incorporation are specifically provided.

1. A gene therapy construct comprising: a nucleic acid encoding one or more tissue regeneration enhancer elements (TREEs) operatively linked to a promoter.

2. The gene therapy construct according to claim 1 in which the gene therapy construct further comprises a nucleic acid sequence comprising one or more pro- or anti-regenerative factors, the expression of which is under the influence of the tissue regeneration enhancer elements.

3. The gene therapy construct according to claim 1 in which the gene therapy particle comprises a vector system.

4. The gene therapy construct according to claim 3 in which the vector system comprises an AAV vector system.

5. The gene therapy construct according to claim 4 in which the gene therapy construct further comprises a first and second AAV inverted terminal repeat (ITR) sequence flanking the sequence encoding the TREEs.

6. The gene therapy construct according to claim 5 in which the ITR nucleotide sequences are derived from AAV serotype 2 (AAV-2).

7. The gene therapy construct according to claim 1 in which the TREE comprises the lepb-linked regulatory enhancer element (LEN).

8. A pharmaceutical composition comprising the gene therapy construct according to claim 1 in a biocompatible pharmaceutical carrier.

9. A method of treating or ameliorating tissue repair comprising administering to a subject a therapeutically effective amount of a gene therapy construct according to claim 1 such that the tissue repair is treated or ameliorated.

10. A method of augmenting tissue regeneration in a subject comprising administering to the subject a therapeutically effective amount of a gene therapy construct according to claim 1 such that the tissue regeneration is augmented.

11. A method of delivering cell therapy to a subject comprising inserting within the genome of reprogrammed stem/progenitor cells of the subject a gene therapy construct according to claim 1 such that cell therapy is delivered.

12. A method for screening drugs that modulate regenerative capacity comprising transfecting a gene construct according to claim 1 into a model system, administering to the system a drug of interest, and measuring regenerative capacity in response to the drug.

13. The method according to claim 12 in which the model system comprises a zebrafish model system.

14. (canceled)


SEQUENCE LISTING (69 sequences)

SEQ ID NO: 1 - DNA, 31 bp, Artificial Sequence
  Synthetic primer
cggaagctttcagcaacttaagtgcattgat

SEQ ID NO: 2 - DNA, 33 bp, Artificial Sequence
  Primer sequence
cccggatccatttctgcaaaagaccaaatgaaa

SEQ ID NO: 3 - DNA, 30 bp, Artificial Sequence
  Primer sequence
cgcggccgccaacgatttaagccccttcat

SEQ ID NO: 4 - DNA, 28 bp, Artificial Sequence
  Primer sequence
ccggtaccactgcaaatcccatcaaaaa

SEQ ID NO: 5 - DNA, 33 bp, Artificial Sequence
  Primer sequence
cccgaattcatttctgcaaaagaccaaatgaaa

SEQ ID NO: 6 - DNA, 34 bp, Artificial Sequence
  Primer sequence
cccggatcctcttgaacaagtgactttcgttgca

SEQ ID NO: 7 - DNA, 33 bp, Artificial Sequence
  Primer sequence
cggaagcttcttgtatgtttatacaagacacat

SEQ ID NO: 8 - DNA, 33 bp, Artificial Sequence
  Primer sequence
cggaagcttactcgccaatttgcttctgtttct

SEQ ID NO: 9 - DNA, 33 bp, Artificial Sequence
  Primer sequence
ggcggatcctggcatacacagcaaacatcatga

SEQ ID NO: 10 - DNA, 30 bp, Artificial Sequence
  Primer sequence
cggaagcttagcagactttgaacccacaga

SEQ ID NO: 11 - DNA, 30 bp, Artificial Sequence
  Primer sequence
cccggatcctctgtgggttcaaagtctgct

SEQ ID NO: 12 - DNA, 28 bp, Artificial Sequence
  Primer sequence
ggaagcttactgagaaggacaggaagct

SEQ ID NO: 13 - DNA, 29 bp, Artificial Sequence
  Primer sequence
cccggatccagcttcctgtccttctcagt

SEQ ID NO: 14 - DNA, 30 bp, Artificial Sequence
  Primer sequence
cggaagcttaggaaattgctgcgtttccca

SEQ ID NO: 15 - DNA, 30 bp, Artificial Sequence
  Primer sequence
cccggatcctgggaaacgcagcaatttcct

SEQ ID NO: 16 - DNA, 34 bp, Artificial Sequence
  Primer sequence
cccggatccacgcgtttggtttttcataaaccac

SEQ ID NO: 17 - DNA, 34 bp, Artificial Sequence
  Primer sequence
cggaagctttcttatttttcagcattgtccttca

SEQ ID NO: 18 - DNA, 32 bp, Artificial Sequence
  Primer sequence
cggaagcttaaatcacatcattccccgtctct

SEQ ID NO: 19 - DNA, 33 bp, Artificial Sequence
  Primer sequence
cccggatcctgttttctacaccagaagagttca

SEQ ID NO: 20 - DNA, 31 bp, Artificial Sequence
  Primer sequence
cccgaattcggagaagacattggaagagcct

SEQ ID NO: 21 - DNA, 34 bp, Artificial Sequence
  Primer sequence
ggcggatcctgtaaatgagctctccaaatcagca

SEQ ID NO: 22 - DNA, 39 bp, Artificial Sequence
  Primer sequence
cccggatcccttaatatggcaataaacgatcttcagaga

SEQ ID NO: 23 - DNA, 35 bp, Artificial Sequence
  Primer sequence
cccgaattcacagtacaaagatccccaaatgatgt

SEQ ID NO: 24 - DNA, 23 bp, Artificial Sequence
  Primer sequence
acaggatacgaatcattgctcga

SEQ ID NO: 25 - DNA, 23 bp, Artificial Sequence
  Primer sequence
tcagagaatgaatgtctcagcca

SEQ ID NO: 26 - DNA, 20 bp, Artificial Sequence
  Primer sequence
agagctacgagctgcctgac

SEQ ID NO: 27 - DNA, 20 bp, Artificial Sequence
  Primer sequence
taccgcaagattccataccc

SEQ ID NO: 28 - DNA, 21 bp, Artificial Sequence
  Primer sequence
ggatacgaatcattgctcgaa

SEQ ID NO: 29 - DNA, 21 bp, Artificial Sequence
  Primer sequence
tctggagacatctggaagtgc

SEQ ID NO: 30 - DNA, 22 bp, Artificial Sequence
  Primer sequence
cacaaatgagttcacatcacca

SEQ ID NO: 31 - DNA, 20 bp, Artificial Sequence
  Primer sequence
tctgctttgccattactcca

SEQ ID NO: 32 - DNA, 29 bp, Artificial Sequence
  Primer sequence
gaattcatgataatgaagaccacgctgct

SEQ ID NO: 33 - DNA, 30 bp, Artificial Sequence
  Primer sequence
ccccaattgagagctgtgcattttggccag

SEQ ID NO: 34 - DNA, 29 bp, Artificial Sequence
  Primer sequence
ccccaattgatgggtgcagtcggcgagct

SEQ ID NO: 35 - DNA, 30 bp, Artificial Sequence
  Primer sequence
cctctagatcagctgtgacctagaacatcc

SEQ ID NO: 36 - DNA, 29 bp, Artificial Sequence
  Primer sequence
gccgaattcatggctgaggtgaaagcagg

SEQ ID NO: 37 - DNA, 29 bp, Artificial Sequence
  Primer sequence
cctctagatcacacagctataggatcctg

SEQ ID NO: 38 - DNA, 30 bp, Artificial Sequence
  Primer sequence
ccggtacccagggggatcatctaatcaagc

SEQ ID NO: 39 - DNA, 36 bp, Artificial Sequence
  Primer sequence
ctggggcccgcagtgaaaaaaatgctttatttgtga

SEQ ID NO: 40 - DNA, 22 bp, Artificial Sequence
  Primer sequence
agagattgatttcggccctgac

SEQ ID NO: 41 - DNA, 22 bp, Artificial Sequence
  Primer sequence
cttgcatgtgccattgtgtttt

SEQ ID NO: 42 - DNA, 28 bp, Artificial Sequence
  Primer sequence
ccactagtcaggccagtcttcagtcacg

SEQ ID NO: 43 - DNA, 34 bp, Artificial Sequence
  Primer sequence
cccggatcccgttggattgatacattcacttcga

SEQ ID NO: 44 - DNA, 29 bp, Artificial Sequence
  Primer sequence
cgcggccgctgtgctttgtaaggctgcac

SEQ ID NO: 45 - DNA, 28 bp, Artificial Sequence
  Primer sequence
ccggtaccgtgctggtgcttcagaatga

SEQ ID NO: 46 - DNA, 44 bp, Artificial Sequence
  Primer sequence
aggaaagtttatgtgaatgcctttctaaaaaaaagttttcttca

SEQ ID NO: 47 - DNA, 45 bp, Artificial Sequence
  Primer sequence
actttaatgagatgaaattgaagaaaacttttttttagaaaggca

SEQ ID NO: 48 - DNA, 29 bp, Artificial Sequence
  Primer sequence
ggcggatccttcgggcttgttgaaagggt

SEQ ID NO: 49 - DNA, 31 bp, Artificial Sequence
  Primer sequence
ccactagtaccatatcttgatacacgccaca

SEQ ID NO: 50 - DNA, 30 bp, Artificial Sequence
  Primer sequence
ggcggatcctgacttcccaagccaaaacct

SEQ ID NO: 51 - DNA, 30 bp, Artificial Sequence
  Primer sequence
cggaagctttgggagcagaagtagaaacgt

SEQ ID NO: 52 - DNA, 32 bp, Artificial Sequence
  Primer sequence
ggcggatccacataccagtgaacacactcact

SEQ ID NO: 53 - DNA, 30 bp, Artificial Sequence
  Primer sequence
cggaagctttctcctcagttctccagatgt

SEQ ID NO: 54 - DNA, 30 bp, Artificial Sequence
  Primer sequence
ggcggatccacagcatgtttgggatgtggt

SEQ ID NO: 55 - DNA, 29 bp, Artificial Sequence
  Primer sequence
cggaagcttgctctgaggtgacagtgctt

SEQ ID NO: 56 - DNA, 32 bp, Artificial Sequence
  Primer sequence
ggcggatcctctaggtacaaatggctctttga

SEQ ID NO: 57 - DNA, 25 bp, Artificial Sequence
  Primer sequence
taaaaaaaaacaagcagaggtatta

SEQ ID NO: 58 - DNA, 53 bp, Artificial Sequence
  Primer sequence
aagattaccaaaataaacttattttaaaactttttattttttattttataata

SEQ ID NO: 59 - DNA, 37 bp, Artificial Sequence
  Primer sequence
aagattaaaactcaaaataaaagccaaggttttatga

SEQ ID NO: 60 - DNA, 34 bp, Artificial Sequence
  Primer sequence
agttttccttcaacttttatttcattaaagtata

SEQ ID NO: 61 - DNA, 25 bp, Artificial Sequence
  Primer sequence
caggaaagtttatgtaaatgacttt

SEQ ID NO: 62 - DNA, 28 bp, Artificial Sequence
  Primer sequence
tttcacttctcattaaagtatatctgac

SEQ ID NO: 63 - DNA, 31 bp, Artificial Sequence
  Primer sequence
atctcattaaagtatgatcttatagacaatt

SEQ ID NO: 64 - DNA, 30 bp, Danio rerio
ctacagcaggtccagatagacttagagact

SEQ ID NO: 65 - DNA, 46 bp, Danio rerio
ctacagcaggtagacttagaattagaacaggatagacttagagact

SEQ ID NO: 66 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 67 - protein, 146 aa, Mus musculus
VPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 68 - protein, 147 aa, Danio rerio
PTAPEDRIRIIARTTISRIKKIKDEHFQMSPEIDFGPDIDNPIDGLSSVLSYLSYLQLRLHVPPAQHLQQVQIDLETLLRTLEELAVSQGCPLPNPETPVHKEETAFPVTSNYLHLLELQRFLEKLCLNIDKLKYCKDTDVAETFIL

SEQ ID NO: 69 - protein, 106 aa, Danio rerio
PTAPEDRIRIIARTTISRIKKIKDEHFQMSPEIDFGPDIDNPIDGLSSVLSYLSYLQLRLHVPPAQHLQQVDLELEQDRLRDSPEDTGGTGRLTGMPSTQSRDPGA